(19) United States
(12) Patent Application Publication
(10) Pub. No.: US 2022/0401592 A1
(43) Pub. Date: Dec. 22, 2022

(54) LOW MOLECULAR WEIGHT AND POLYAMIDOAMINE (PAMAM) DENDRIMER BASED PSMA-SPECIFIC DUAL CONTRAST AGENTS FOR OPTICAL AND PHOTOACOUSTIC IMAGING AND THERANOSTIC AGENTS FOR TREATING PROSTATE CANCER

(71) Applicant: The Johns Hopkins University, Baltimore, MD (US)

(72) Inventors: Martin G. Pomper, Baltimore, MD (US); Wojciech Lesniak, Owings Mills, MD (US); Emad Boctor, Ellicott City, MD (US); Sangeeta Banerjee Ray, Ellicott City, MD (US); Srikanth Boinapally, Baltimore, MD (US); Jeeun Kang, Baltimore, MD (US); Yixuan Wu, Baltimore, MD (US)

(21) Appl. No.: 17/744,080

(22) Filed: May 13, 2022

Related U.S. Application Data
(60) Provisional application No. 63/188,831, filed on May 14, 2021.

(51) Int. Cl. A61K 51/04; A61K 49/00; A61K 47/54; A61K 47/60

(52) U.S. Cl. CPC A61K 51/0482; A61K 49/0032; A61K 47/547; A61K 47/60

(57) ABSTRACT

Poly(amidoamine) [PAMAM] dendrimers for use as PSMA-targeted contrast agents for optical and photoacoustic imaging (PA) and theranostic agents for treating prostate cancer are disclosed.

### STATEMENT OF GOVERNMENTAL INTEREST

This invention was made with government support under grants CA134675, CA184228, CA183031, EB024495 awarded by the National Institutes of Health, and W81XWH-18-1-0188 awarded by the Department of Defense.

### BACKGROUND

Prostate cancer (PC) is the second most common malignancy in men and the fifth leading cause of cancer-related death, with more than 1.1 million new cases diagnosed annually worldwide, which imposes significant socioeconomic burdens. Ferlay et al., 2015. While patients with indolent PC may be cured, patients with aggressive PC often have poor prognosis and thus require aggressive treatments, involving systemic androgen deprivation therapy accompanied by chemotherapy and radiation therapy. Damodaran et al., 2019; James et al., 2016. Early detection of aggressive PC and selection of appropriate therapy increase patient's chance to be cured.

While elevated blood level of prostate-specific antigen (PSA) allows early detection of PC, recent studies have indicated that its utility for patient stratification for aggressive treatment may be insufficient. Pinsky et al., 2017. Thus, the increased PSA blood level may be rather used as the indication for a more definitive ultrasound guided transrectal or transperineal needle biopsy, which also is problematic because of high-error rate due to random sampling and possible side effects, such as pain, bleeding and infection. Tosoian et al., 2011. Despite these shortcomings, PSA testing and serial prostate biopsies are widely utilized for active surveillance of PC. Chen et al., 2016; Mamawala et al., 2017. Thus, there is an urgent need for development of less invasive and more accurate methods for active surveillance and treating patients with PC.

### SUMMARY

In some aspects, the presently disclosed subject matter provides a compound of formula (I):

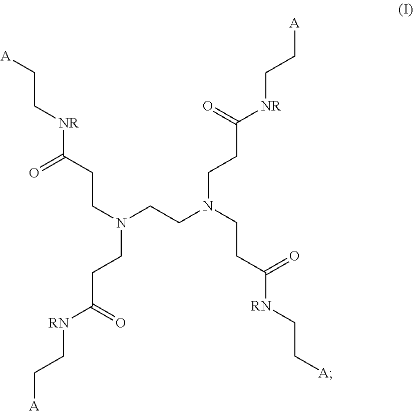

wherein: each A is:

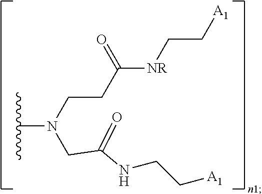

wherein each A₁ is selected from A, a prostate-specific membrane antigen (PSMA) targeting moiety, an optical imaging agent, a chelating agent, and/or a therapeutic agent, and an end-capping group (EC) or —NH₂; n1 is an integer selected from 1, 2, 3, 4, 5, 6, 7, 8, 9, and 10; and pharmaceutically acceptable salts thereof.

In certain aspects, the PSMA targeting moiety comprises a Lys-Glu-urea moiety having the following structure:

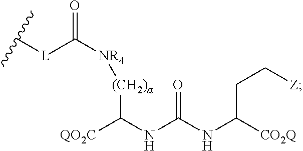

wherein: Z is tetrazole or CO₂Q; Q is H or a protecting group; a is an integer selected from 1, 2, 3, 4, and 5; R₄ is independently H, substituted or unsubstituted C₁-C₄ alkyl, or —CH₂-R₅; R₅ is selected from substituted or unsubstituted aryl and substituted or unsubstituted heteroaryl; and L is a linker.

In particular aspects, the linker (L) is selected from —(CH₂)ₘ₁—, —C(═O)—(CH₂)ₘ₁—, —NR₃—C(═O)-(CH₂)ₘ₁—, —(CH₂—CH₂—O)ₜ₁—, —C(═O)-(CH₂—CH₂—O)ₜ₁—, —(O—CH₂—CH₂)ₜ₁—,—C(═O)-(O—CH₂—CH₂)ₜ₁—, —C(═O)-(CHR₂)ₘ₁—NR₃—C(═O)-(CH₂)ₘ₁—, —C(═O)-(CH₂)ₘ₁—O—C(═O)—NR₃—(CH₂)ₚ₁—, —C(═O)-(CH₂)ₘ₁—NR₃—C(═O)—O—CH₂)ₚ₁—, —C(═O)-(CH₂)ₘ—NR₃—C(═O)—NR₃—(CH₂)ₚ—, —C(═O)-(CH₂)ₘ—NR₃—C(═O)-(CH₂)ₚ₁—, —C(═O)-(CH₂)ₘ₁—C(═O)—NR₃—(CH₂)ₚ₁—, —C(═O)-(CH₂)ₘ₁—NR₃—C(═O)—NR₃—(CH₂)ₚ₁—, —C(═O)-(CH₂)ₘ₁—NR₁—C(═O)—NR₃—(CH₂)ₚ₁—, —C(═O)-(CH₂)ₘ₁—O—C(═O)—NR₃—,—C(═O)—CH₂)ₘ₁—O—C(═O)—NR₃—(CH₂)ₚ₁—,—C(═O)-(CH₂)ₘ₁—NR₃—C(═O)—O—(CH₂)ₚ₁—, polyethylene glycol, glutaric anhydride, albumin, and one or more amino acids: we each R is independently selected from H and C₁-C₄ alkyl; each R₁ is independently selected from H, Na⁺, C₁-C₄ alkyl, and a protecting group; each R₂ is independently selected from hydrogen, and —COOR₁; each R₃ is independently selected from hydrogen, substituted or unsubstituted linear or branched alkyl, alkoxyl, substituted or unsubstituted cycloalkyl, substituted or unsubstituted cycloheteroalkyl, substituted or unsubstituted aryl, substituted or unsubstituted heteroaryl, substituted or unsubstituted arylalkyl, and substituted or unsubstituted heteroarylalkyl; m1 and p1 are each independently an integer selected from 0, 1, 2, 3, 4, 5, 6, 7 and 8; t1 is an integer selected from 1, 2, 3, 4, 5, 6, 7, 8, 9, 10, 11 and 12. In some embodiments, the linker (L) comprises —NH—C(═O)-(CH₂)ₘ₁, wherein m₁ is selected from 1, 2, 3, 4, 5, 6, 7, and 8.

In certain aspects, the optical imaging agent (IA) comprises a fluorescent dye. In particular aspects, the fluorescent dye comprises a near-infrared dye, including, but not limited to, a cyanine dye, or derivative thereof. In particular aspects, the optical imaging agents is selected from:

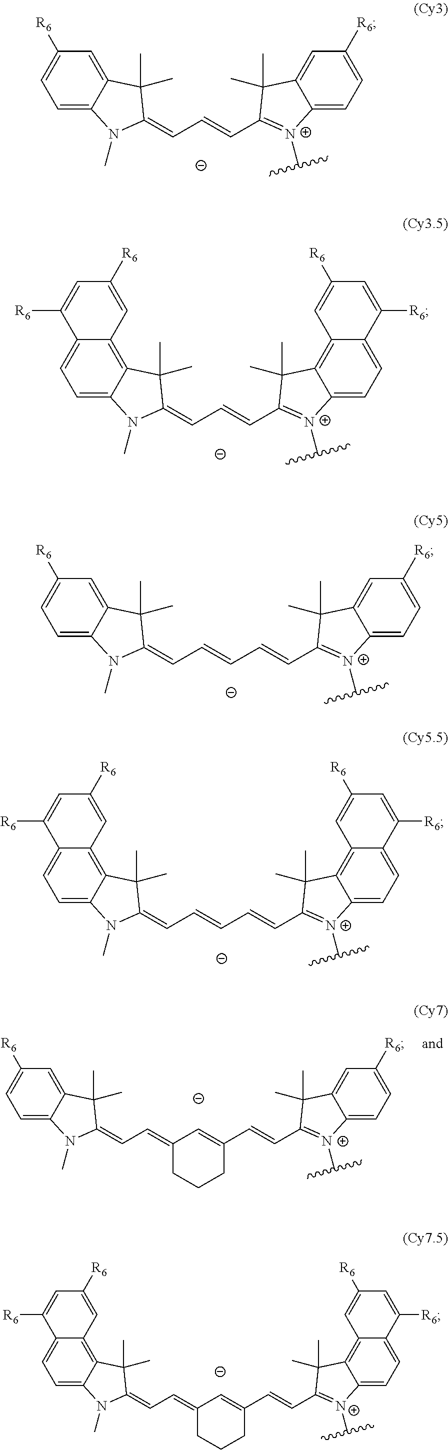

wherein each R₆ can independently be H or —SO₃M, wherein is absent, e.g., R₆ is —SO₃—, hydrogen, sodium, or potassium.

In particular aspects, the fluorescent dye is Cy7.5:

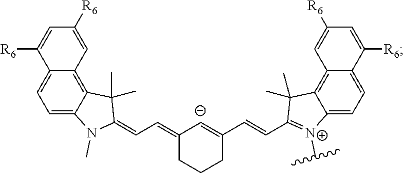

wherein each R₆ can independently be H or —SO₃M, wherein is absent, e.g., R₆ is —SO₃—, hydrogen, sodium, or potassium.

In some aspects, the chelating agent comprises NOTA.

In some aspects, the chelating agent further comprises a radiometal selected from of ⁶⁰Cu, ⁶²Cu, ⁶⁴Cu, ⁶⁷Cu, ²⁰³Pb, ²¹²Pb, ²²⁵Ac, ¹⁷⁷Lu, ^{99m}Tc ⁶⁸Ga¹⁴⁹Tb, ⁸⁶y, ⁹⁰y, ¹¹¹In ¹⁸⁶Re, ¹⁸⁸Re, ¹⁵³Sm, ⁸⁹Zr, ²¹³Bi, ²¹²Bi, ²¹²Pb, ⁶⁷Ga ⁴⁷Sc, Al¹⁸F, and ¹⁶⁶Ho. In particular aspects, the radiometal is selected from ⁶⁴Cu and ⁶⁷Cu.

In some aspects, the therapeutic agent comprises an anti-cancer or chemotherapeutic agent. In particular aspects, the therapeutic agent is selected from maytansine, ansamitocin, mertansine/emtansine (DM1), ravtansine/soravtansine (DM4), monomethyl auristatin e, methotrexate, doxorubicin, and paclitaxel.

In some aspects, the end-capping group (EC) is selected from —NH₂, —(CH₂)ₘ₁—CH₂—CH(OR₁)(CH₂)ₘ₁—OR₁, —NR—(CH₂)ₘ₁—CH(OR₁)-(CH₂)ₘ₁—OR₁, —NR—C(═O)—CH₃, —C(═O)—O—Na⁺, —C(═O)—NR—(CH₂)ₘ₁—OR₁, —NR—C(═O)-(CH₂)ₘ₁—C(═O)OR₁, and —NR—(CH₂)ₘ₁—CH(OR₁)-(CH₂)ₘ₁—CH₃; wherein: each R is independently selected from H and C₁-C₄ alkyl; each R₁ is independently selected from H, Na⁺, C₁-C₄ alkyl, and a protecting group; and each m₁ is independently an integer selected from 0, 1, 2, 3, 4, 5, 6, 7, 8, 9, 10, 11, and 12. In particular embodiments, the end-capping group is —NH—CH₂—CH(OH)—CH₂—OH.

In certain aspects, the compound of formula (I) comprises a PAMAM dendrimer having the following chemical structure:

wherein: m, n, p, q, and t are each independently integers from 0 to 64; EC is an end-capping group; IA is an optical imaging agent; and PSMA is a PSMA-targeting moiety.

In particular aspects, the compound of formula I is:

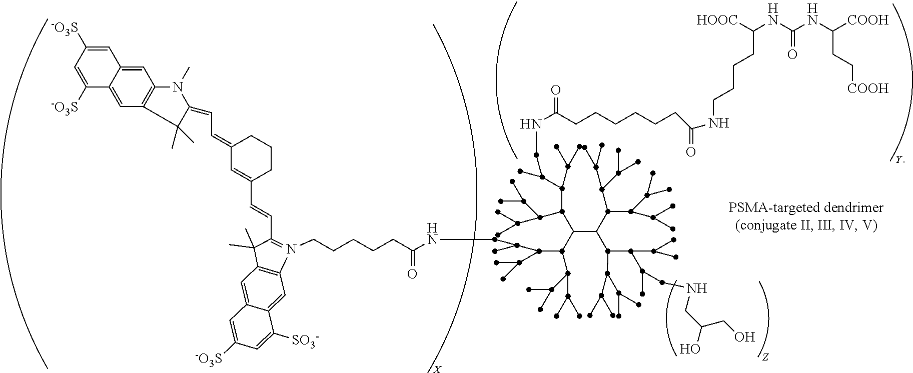

In some aspects, X is selected from 1, 2, 3, 4, 5, 6, 7, 8, 9, 10, 11, 12, 13, 14, 15, 16, 17, 18, 19, and 20; Y is selected from 1, 2, 3, 4, 5, 6, 7, 8, 9, 10, 11, 12, 13, 14, 15, 16, 17, 18, 19, and 20; and Z is an integer between about 20 and 60.

In aspects embodiments, the presently disclosed subject matter provides a compound of formula (II):

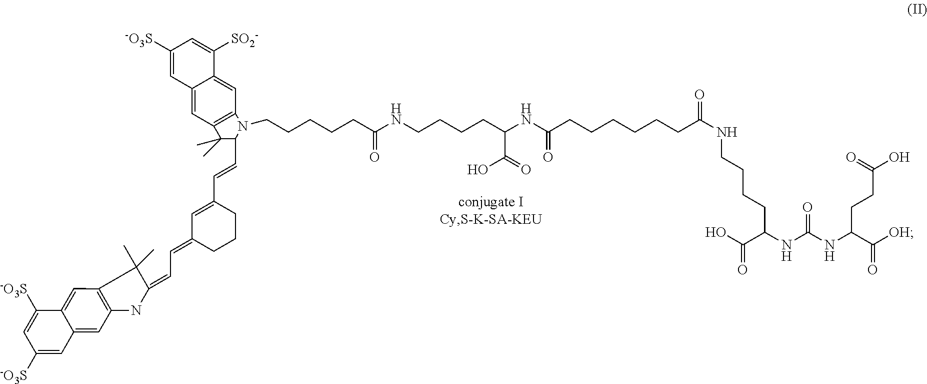

and pharmaceutically acceptable salts thereof.

In some aspects, the presently disclosed subject matter provides a pharmaceutical formulation comprising the compound of formula (I) or formula (II) and a pharmaceutically acceptable carrier, diluent, or excipient.

In other aspects, the presently disclosed subject matter provides a method for imaging and/or treating one or more PSMA-expressing expressing tumors or cells, the method comprising contacting the one or more PSMA expressing tumors or cells with an effective amount of a compound of formula (1) or formula (II), or a pharmaceutical formulation thereof and, if a method for imaging, taking an image.

In particular aspects, the imaging is selected from optical imaging, photoacoustic imaging, and positron emission tomography/computed tomography (PET/CT) imaging.

In certain embodiments, the imaging is in vitro, in vivo, or ex vivo. In some embodiments, the method further comprises diagnosing and/or treating, based on the image, a disease or condition in a subject. In some embodiments, the method further comprises monitoring, based on the image, progression or regression of a disease or condition in a subject.

In certain aspects, the method comprises imaging a cancer. In particular aspects, the cancer is selected from prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature. In particular embodiments, the cancer comprises prostate cancer.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one figure executed in color. Copies of this patent or patent application publication with color figures will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B show the synthesis of representative presently disclosed compounds. FIG. 1A—Synthesis of PSMA-targeting moiety NHS-SA-KEU (n-hydroxysuccinimide ester-suberic acid-lysine-glutamate-urea) for conjugation with dendrimer and PSMA-targeted low molecular weight (LMW) Cy7.5-K-SA-KEU conjugate I (sulfo-cyanine7.5-lysine-suberic acid-lysine-glutamate-urea); FIG. 1B-scheme demonstrating formulation of PSMA-targeted dendrimers (conjugate II-generation 4 PAMAM dendrimer conjugated with two Cy7.5 dyes, ten SA-KEU targeting agents and capped with thirty-two butane-1,2-diol moieties, conjugate III-generation 4 PAMAM dendrimer conjugated with four Cy7.5 dyes, nine SA-KEU targeting agents and capped with thirty butane-1,2-diol moieties, conjugate IV-generation 4 PAMAM dendrimer conjugated with six Cy7.5 dyes, nine SA-KEU targeting agents and capped with fifty-three butane-1,2-diol moieties, conjugate V-generation 4 PAMAM dendrimer conjugated with eight Cy7.5 dyes, twelve SA-KEU targeting agents and capped with fifty-two butane-1,2-diol moieties) and conjugate VI (control dendrimer)—generation 4 PAMAM dendrimer conjugated with six Cy7.5 dyes and capped with fifty-two butane-1,2-diol moieties;

FIG. 2A is a RP-HPLC chromatogram of C7.5-K-SA-KEU (conjugate I) with the UV-Vis spectrum recorded under the peak (insert), indicating covalent attachment of Cy7.5 to the K-SA-KEU moiety;

FIG. 2B is a MALDI-TOF spectrum of conjugate I, confirming its identity with molecular mass of 1554 Da;

FIG. 3A, FIG. 3B, FIG. 3C, FIG. 3D, and FIG. 3E show the physicochemical characterization of the presently disclosed compounds. FIG. 3A shows representative MALDI-TOF MS spectra illustrating increase of the molecular weight upon each modification step of dendrimer's terminal primary amines, (G4(NH₂)₆₄—generation 4 amine terminated PAMAM dendrimer used as starting material and consecutive conjugates obtained through conjugation of 6 sulfo-Cy7.5 dyes (2), 9 SA-KEU PSAM targeting agents (3) and capping of primary amines with butane-1,2-diol moieties (4-final PSMA-targeted dendrimer conjugate IV); FIG. 3B is a RP-HPLC chromatogram of conjugate IV with the UV-Vis spectrum recorded under the peak, indicating covalent attachment of sulfo-Cy7.5 to the dendrimer; FIG. 3C shows the spectrophotometric properties of all conjugates and purified water from 650 nm to 950 nm in step of 1 nm;

FIG. 3D shows the spectrofluorometric properties of all conjugates and purified water. The excitation was at 715 nm and the emission was from 750 nm to 850 nm in step of 1 nm; and FIG. 3E shows the PA properties of all conjugates from 700 nm to 900 nm in step of 10 nm. From FIG. 3C to FIG. 3E all intensities are normalized with respective to conjugate I—LMW contrast agent with single sulfo-Cy7.5 dye;

FIG. 4A, FIG. 4B, and FIG. 4C show in vitro evaluation. FIG. 4A shows conjugate IV binding to PSMA+PC3 PIP and PSMA-PC3 flu cell lines; FIG. 4B shows evaluation of conjugate IV binding affinity to PSMA+PC3 PIP cells (approximately 1×10⁶ PSMA+PC3 PIP cells incubated with varied concentration of conjugate IV); and FIG. 4C microscopic making of cellular internalization of conjugate IV in PSMA+PC3 PIP and PSMA-PC3 flu cell lines;

FIG. 5A, FIG. 5B, and FIG. 5C shows optical imaging data of representative presently disclosed compounds. FIG. 5A shows NOD-SCID mice bearing subcutaneous PSMA+PC3 PIP and PSMA-PC3 flu in the lower back near left and right posterior flanks, respectively were injected with conjugate IV and optical images were acquired in the 800 nm NIR and white light channels 5, 24, 48 and 72 h after injection; representative image of ex vivo biodistribution of conjugate IV at 72 h after injection and semi-quantitative analysis of fluorescence intensity (column numbers in the graph represent the tissue numbers, n=5); FIG. 5B shows the ex vivo evaluation of conjugate IV uptake in PSMA+PC3 PIP and PSMA-PC3 tumors in sections throughout entire volume of the tumors, scanned using a Li-Cor Odyssey infrared imaging system; and FIG. 5C shows distribution of conjugate IV in PSMA+PC3 PIP and PSMA-PC3 tumors at the microscopic level (scale bar denotes 120 μm). Data indicate preferential accumulation of the PSMA-targeted dendrimer conjugate IV in PSMA+PC3 PIP tumors and extravasation from blood vessels in the entire volume of the tumor with strong optical signal;

FIG. 6 shows representative optical image of NOD-SCID mice bearing subcutaneous PSMA+PC3 PIP and PSMA-PC3 flu in the lower back near right and left posterior flanks, respectively (as indicated by arrows), injected with non-targeted control conjugate VI. In contrast to PSMA-targeted dendrimer conjugates, control conjugate did not show preferential uptake in PSMA+PC3 PIP tumors. Signal observed in the in vivo images was localized in kidneys as confirmed by ex vivo organs imaging;

FIG. 7 shows the framework of spectral unmixing for spectroscopic PA imaging of PSMA expression. Schematic depiction of the presently disclosed PSMA-targeted imaging agent composed of generation-4 poly(amidoamine) dendrimer conjugated with six Cy7.5 dyes and nine lysine-glutamate-urea PSMA targeting moieties, and remaining terminal primary amines capped with butane-1,2-diol functionalities;

FIG. 8A, FIG. 8B, and FIG. 8C show noise characteristics in spectroscopic PA imaging. (FIG. 8A) Mean, STD, and the coefficient of variance (CV) of the Nd:YAG OPO laser at different wavelengths; (FIG. 8B) Energy distribution of 5,000 sequential laser pulses emitted by an Nd:YAG OPO laser; (FIG. 8C) Background noise intensity histogram (EM 3);

FIG. 9 shows in vivo ultrasound and photoacoustic imaging of conjugate IV and VI. Mice used for NIR optical imaging were also utilized for US and PA imaging.

Images were collected before the conjugates were injected and their accumulation in PSMA+PC3 PIP and PSMA-PC3 flu tumors was measured at 5, 24, 48 and 72 hrs after injection. Image fusion of co-registered US and PA images were implemented in MATLAB, FU—fused;

FIG. 10 shows in vivo optical, ultrasound and photoacoustic imaging of conjugate L All experimental protocols and image analysis methods are the same as used to demonstrate results in FIG. 9;

FIG. 11 shows a comparison of the normalized PA signal contrast between PSMA+PC3 PIP and PSMA-PC3 flu provided by conjugate I, IV and VI in the PSMA+PC3 PIP tumors. Data points in different markers indicate the intensity of different mice (n=5). One, two, and three asterisks correspond to P-values <0.05, <0.01, and <0.001, respectively;

FIG. 12A and FIG. 12B show: FIG. 12A—Synthesis of PSMA-targeting moiety terminated with amine reactive NHS ester (the same as for FL and contrast agents); FIG. 12B—Modification of DM1 with disulfide cleavable and NHS ester containing linker for conjugation with a representative dendrimer;

FIG. 13 illustrates the formulation of PSMA-targeted dendrimer-drug conjugates (PT-DDC) by consecutive conjugation of DM1, NOTA, Cy5, SA-KEU and capping remaining primary amine with butane-1,2-diol groups. Also shown are MADLI-TOF MS data indicating successful conjugation of each functional moiety with the dendrimer by showing increase of the molecular weight upon each modification step that was used to calculation of average number of DM1, NOTA, Cy5, SA-KEU and butane-1,2-diol molecules conjugated with dendrimer that are included in the scheme;

FIG. 14 illustrates the formulation of control dendrimer-drug conjugates (Ctrl-DDC) by consecutive conjugation of DM1, NOTA, Cy5 and capping remaining primary amine with butane-1,2-diol groups. Also shown are MADLI-TOF MS data indicating successful conjugation of each functional moiety with the dendrimer by showing increase of the molecular weight upon each modification step that was used to calculation of average number of DM1, NOTA, Cy5 and butane-1,2-diol molecules conjugated with dendrimer that are included in the scheme;

FIG. 15A, FIG. 15B, FIG. 15C, and FIG. 15D show in vitro evaluation of PT-DDCs. FIG. 15A—Binding of PT-DDCs and Ctrl-DDCs to PSMA+PC3 PIP and PSMA-PC3 flu cells in the absence and presence of ZJ-43; FIG. 15B—Evolution of PT-DDCs affinity to PSMA+PC3 PIP; FIG. 15C—Imaging of PT-DDCs binding to PSMA+PC3 PIP and PSMA-PC3 flu cells and FIG. 15D—Assessment of cytotoxicity. Data indicates specific uptake of PT-DDCs by PSMA+PC3 PIP cells leading to enhanced cytotoxicity;

FIG. 16A and FIG. 16B demonstrate the analysis of PT-DDCs stability. FIG. 16A-HPLC chromatograms of PT-DDCs collected after incubation in PBS for 24 h at 37° C. showing presence of only the conjugate and release of DM1 upon addition of glutathione (GHS); FIG. 16B—The same evaluation of PT-DDCs in human blood plasma. Results indicate that PT-DDCs are stable in human blood plasma and DM1 can be release upon cellular internalization in presence of GSH;

FIG. 17A, FIG. 17B, FIG. 17C, FIG. 17D, and FIG. 17E show a therapy study in mice bearing PSMA+PC3 PIP or PSMA-PC3 flu threated with 2.5, 5 and 10 mg/kg of PT-DDCs on day 1 and 11 (n=5). FIG. 17A—Tumor volumes (arrow indicates second administration of PT-DDCs); FIG. 17B—Representative optical imaging of mice treated with 10 mg/kg of PT-DDC; FIG. 17C—Fluorescence intensity detected within tumors in all treatment groups; FIG. 17D—% tumor growth inhibition on day 21; FIG. 17E-Biodistribution of PT-DDCs 10 days after injection of the 2nd dose of 2.5 mg/kg on day 11 of the therapy study in mice bearing PIP tumors. Optical images were collected using Xenogen IVIS Spectrum system with excitation at 610 nm and emission at 660 nm. Results indicate dose dependent accumulation of PT-DDCs in PSMA+PC3 PIP tumors and therapeutic response; and FIG. 18A and FIG. 18B are: FIG. 18A—Representative decay-corrected, volume-rendered PET/CT images of mice bearing PSMA+PC3 PIP and PSMA-PC3 flu tumors injected with 5 mg/kg (approximately 100 μg) of PT-DDCs and 16.65 MBq (450 μCi) of [⁶⁴Cu] PT-DDCs and their biodistribution at 72 h after injection (n=3); FIG. 18B-Representative optical imaging of tissues dissected from mice used in PET-CT imaging, demonstrating biodistribution of PT-DDCs at 72 h after administration. Optical images were collected using Xenogen IVIS Spectrum system with excitation at 610 nm and emission at 660 nm. After completion of optical imaging tissue samples were weighted and transfer to auto-gamma counter to evaluate biodistribution of [⁶⁴Cu] PT-DDCs presented in the panel FIG. 18A (green bars). Results indicate feasibility of concurrent PSMA-specific chemo- and radiotherapy by PT-DDCs.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Compounds of Formula (I)

In some embodiments, the presently disclosed subject matter provides a poly(amidoamine) (PAMAM) dendrimer comprising one or more prostate-specific membrane antigen (PSMA) targeting moieties, one or more optical imaging agents, chelating agents, and/or therapeutic agents, and one or more end-capping groups, wherein the one or more prostate-specific membrane antigen (PSMA) targeting moieties and the one or more optical imaging agents are operably linked to the PAMAM dendrimer; and pharmaceutically acceptable salts thereof.

In some embodiments, the PAMAM dendrimer is a compound of formula (I):

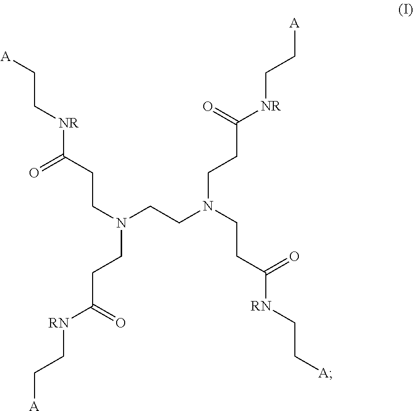

wherein: each A is:

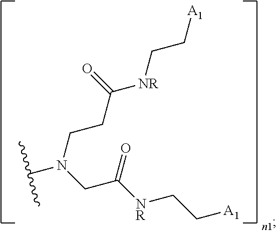

wherein each A₁ is selected from A, a prostate-specific membrane antigen (PSMA) targeting moiety, an optical imaging agent (IA), and an end-capping group (EC) or —NH₂; n1 is an integer selected from 1, 2, 3, 4, 5, 6, 7, 8, 9, and 10; and pharmaceutically acceptable salts thereof.

In particular embodiments, the PAMAM dendrimer is a generation four (G4) PAMAM dendrimer. Other PAMAM dendrimers of generation 0, 1, 2, 3, 4, 5, 6, 7, 8, 9, and 10 also are suitable for use with the presently disclosed subject matter.

In certain embodiments, the PSMA targeting moiety comprises a Lys-Glu-urea moiety having the following structure:

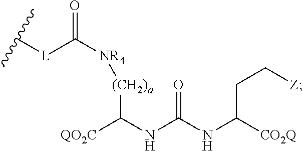

wherein: Z is tetrazole or CO₂Q; Q is H or a protecting group; a is an integer selected from 1, 2, 3, 4, and 5; R₄ is independently H, substituted or unsubstituted C₁-C₄ alkyl, or —CH₂-R₅; R₅ is selected from substituted or unsubstituted aryl and substituted or unsubstituted heteroaryl; and L is a linker.

In particular embodiments, the linker (L) is selected from —(CH₂)ₘ₁—, —C(═O)—(CH₂)ₘ₁—, —NR₃—C(═O)-(CH₂)ₘ₁—, —(CH₂—CH₂—O)ₜ₁—, —C(═O)-(CH₂—CH₂—O)ₜ₁—, —(O—CH₂—CH₂)ₜ₁—,—C(═O)-(O—CH₂—CH₂)ₜ₁—, —C(═O)-(CHR₂)ₘ₁—NR₃—C(═O)-(CH₂)ₘ₁—, —C(═O)-(CH₂)ₘ₁—O—C(═O)—NR₃—(CH₂)ₚ₁—, —C(═O)-(CH₂)ₘ₁—NR₃—C(═O)—O—CH₂)ₚ₁—, —C(═O)-(CH₂)ₘ—NR₃—C(═O)—NR₃—(CH₂)ₚ—, —C(═O)-(CH₂)ₘ—NR₃—C(═O)-(CH₂)ₚ₁—, —C(═O)-(CH₂)ₘ₁—C(═O)—NR₃—(CH₂)ₚ₁—, —C(═O)-(CH₂)ₘ₁—NR₃—C(═O)—NR₃—(CH₂)ₚ₁—, —C(═O)-(CH₂)ₘ₁—NR₁—C(═O)—NR₃—(CH₂)ₚ₁—, —C(═O)-(CH₂)ₘ₁—O—C(═O)—NR₃—,—C(═O)—CH₂)ₘ₁—O—C(═O)—NR₃—(CH₂)ₚ₁—,—C(═O)-(CH₂)ₘ₁—NR₃—C(═O)—O—(CH₂)ₚ₁—, polyethylene glycol, glutaric anhydride, albumin, and one or more amino acids; wherein each R is independently selected from H and C₁-C₄ alkyl; each R₁ is independently selected from H, Na⁺, C₁-C₄ alkyl, and a protecting group; each R₂ is independently selected from hydrogen, and —COOR₁; each R₃ is independently selected from hydrogen, substituted or unsubstituted linear or branched alkyl, alkoxyl, substituted or unsubstituted cycloalkyl, substituted or unsubstituted cycloheteroalkyl, substituted or unsubstituted aryl, substituted or unsubstituted heteroaryl, substituted or unsubstituted arylalkyl, and substituted or unsubstituted heteroarylalkyl; m1 and p1 are each independently an integer selected from 0, 1, 2, 3, 4, 5, 6, 7 and 8; t1 is an integer selected from 1, 2, 3, 4, 5, 6, 7, 8, 9, 10, 11 and 12. In some embodiments, the linker (L) comprises —NH—C(═O)-(CH₂)ₘ₁, wherein m₁ is selected from 1, 2, 3, 4, 5, 6, 7, and 8.

In certain embodiments, the optical imaging agent comprises a fluorescent dye. In more certain embodiments, the fluorescent dye is a near-infrared dye, including, but not limited to, a cyanine dye, or derivative thereof. Representative cyanine dyes include, but are not limited to, a Cy2, Cy3, Cy3.5, Cy5, Cy5.5, Cy7, and Cy7.5 dye, including sulfo-Cy dyes:

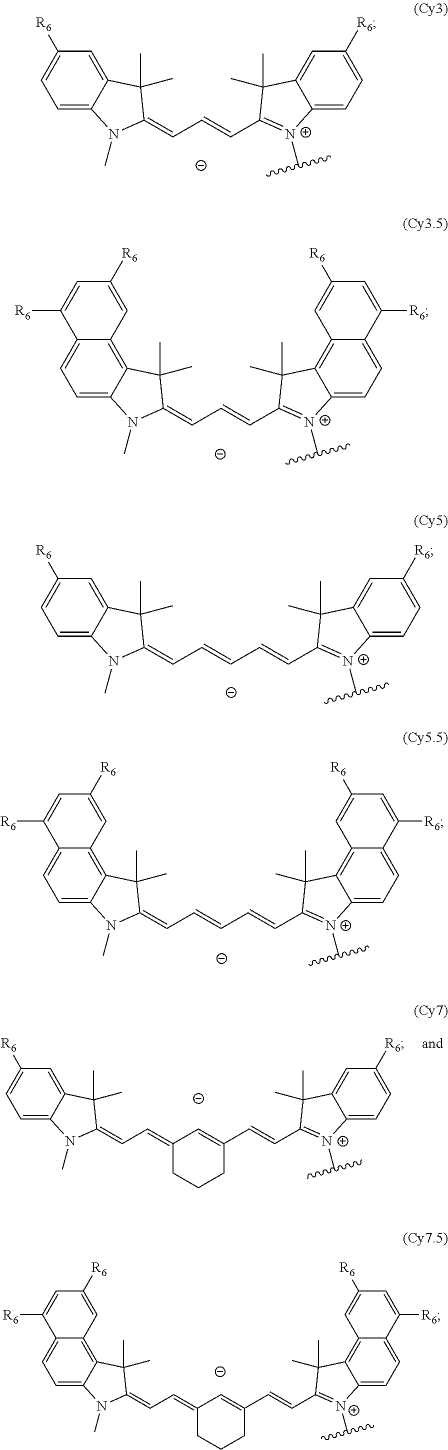

(Cy7.5); wherein each R₆ can independently be H or —SO₃M, wherein is absent, e.g., R₆ is —SO₃—, hydrogen, sodium, or potassium.

In some embodiments, the fluorescent dye is Cy7.5:

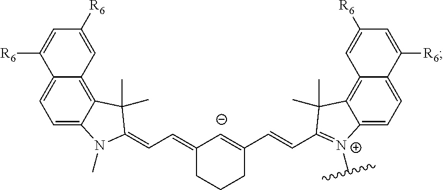

wherein each R₆ can independently be H or —SO₃M, wherein is absent, e.g., R₆ is —SO₃—, hydrogen, sodium, or potassium.

In some embodiments, the imaging agent (IA) is conjugated to the PAMAM dendrimer via a heterobifunctional crosslinker (CL). In certain embodiments, the heterobifunctional crosslinker (CL) is selected from succinimidyl 4-(N-maleimidomethyl)cy clohexane-1-carboxylate (SMCC), m-maleimidobenzoyl-N-hydroxysuccinimide ester (MBS), N-(beta-maleimidopropyloxy)succinimide ester (BMPS), N-[e-maleimidocaproyloxy]succinimide ester (EMCS), N-[gamma-maleimidobutyryloxy] succinimide (GMBS), N-succinimidyl 4-[4-maleimidophenyl]butyrate (SMPB), succinimidyl-6-(β-maleimidopropionamido)hexanoate (SMPH), and maleimide-polyethylene glycol-N-hydroxysuccinimide ester (MAL-PEG-NHS). In other embodiments, the imaging agent (IA) is conjugated to the PAMAM dendrimer via a linker (L) as defined hereinabove. In particular embodiments, the linker (L) comprises —NH—C(═O)-(CH₂)ₘ₁, wherein m₁ is selected from 1, 2, 3, 4, 5, 6, 7, and 8.

In some embodiments, the end-capping group (EC) is selected from —NH₂, —(CH₂)ₘ₁—CH₂—CH(OR₁)-(CH₂)ₘ₁—OR₁, —NR—(CH₂)ₘ₁—CH(OR₁)-(CH₂)ₘ₁—OR₁, —NR—C(═O)—CH₃, —C(═O)—O—Na⁺, —C(═O)—NR—(CH₂)ₘ₁—OR₁, —NR—C(═O)-(CH₂)ₘ₁—C(═O)OR₁, and —NR—(CH₂)ₘ₁—CH(OR₁)-(CH₂)ₘ₁—CH₃; wherein: each R is independently selected from H and C₁-C₄ alkyl; each R₁ is independently selected from H, Na⁺, C₁-C₄ alkyl, and a protecting group; and each m₁ is independently an integer selected from 0, 1, 2, 3, 4, 5, 6, 7, 8, 9, 10, 11, and 12. In particular embodiments, the end-capping group is —NH—CH₂—CH(OH)—CH₂—OH.

In certain embodiments, the PAMAM dendrimer has the following chemical structure:

wherein: m, n, p, q, and t are each independently integers from 0 to 64; EC is an end-capping group; IA is an optical imaging agent; and PSMA is a PSMA-targeting moiety.

In particular embodiments, the compound of formula I is:

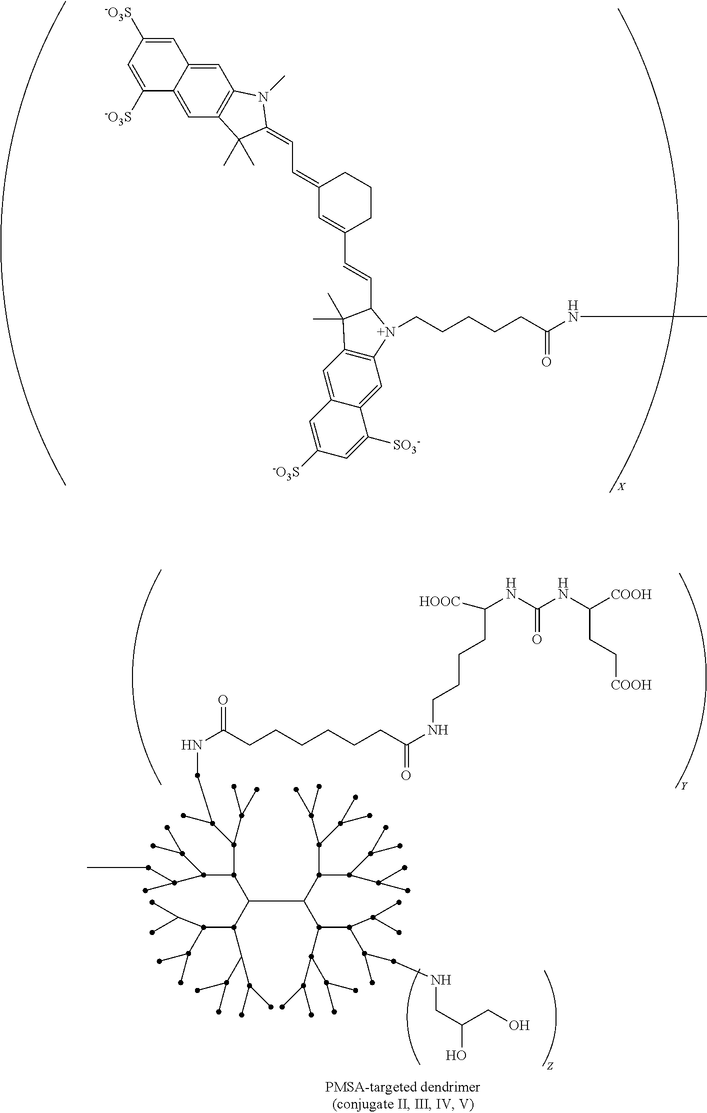

In some embodiments, X is selected from 1, 2, 3, 4, 5, 6, 7, 8, 9, 10, 11, 12, 13, 14, 15, 16, 17, 18, 19, and 20. In particular embodiments, X is selected from 2, 4, 6, and 8. In some embodiments, Y is selected from 1, 2, 3, 4, 5, 6, 7, 8, 9, 10, 11, 12, 13, 14, 15, 16, 17, 18, 19, and 20. In particular embodiments, Y is selected from 9, 10, and 12.

In some embodiments, Z is an integer between about 20 and 60, including, but not limited to, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60. In particular embodiments, Z is selected from 30, 32, 52, and 53.

In other embodiments, the presently disclosed subject matter provides a compound of formula (II):

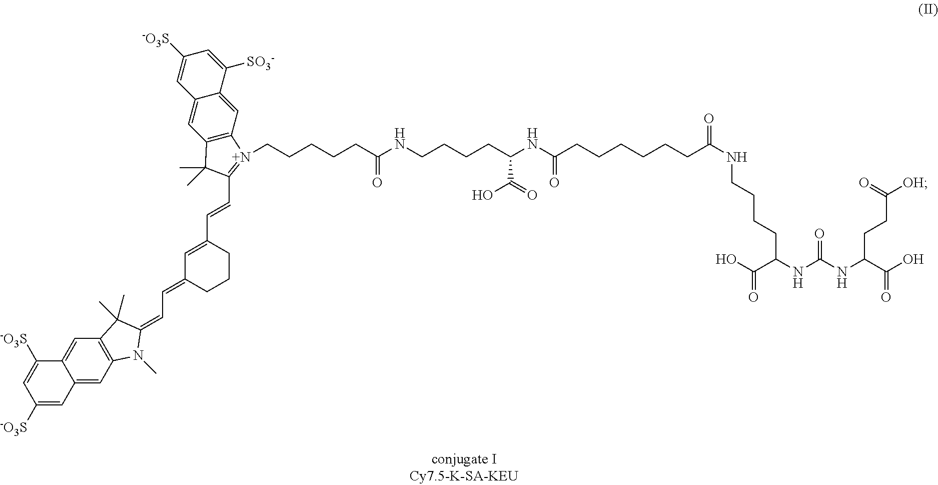

and pharmaceutically acceptable salts thereof.

In some embodiments, the presently disclosed subject matter provides a pharmaceutical formulation comprising the compound of formula (I) or formula (II) and a pharmaceutically acceptable carrier, diluent, or excipient.

In some embodiments, one or more of the Cy7.5 dyes can be replaced with one or more other optical dyes. In certain embodiments, optical dye comprises a fluorescent dye. In yet more certain embodiments, the fluorescent dye comprises a fluorescent dye that emits in the near infrared spectral region. In particular embodiments, the fluorescent dye is selected from a polymethine dye, a coumarin dye, a xanthene dye, and a boron-dipyrromethene (BODIPY) dye.

In certain embodiments, the polymethine dye is selected from a carbocyanine dye, an indocarbocyanine dye, an oxacarbocyanine dye, a thiacarbocyanine dye, and a merocyanine dye. In certain embodiments, the xanthene dye is selected from a fluorescein dye and a coumarin dye. In particular embodiments, the fluorescent dye is selected from:

BODIPY FL, BODIPY R6G, BODIPY TR, BODIPY TMR, BODIPY 493/503, BODIPY 530/550, BODIPY 558/568, BODIPY 564/570, BODIPY 576/589, BODIPY 581/591,BODIPY 630/650, and BODIPY 650/665;

VivoTag-645, VivoTag-680, VivoTag-S680, VivoTag-S750, VivoTag-800;

Alexa Fluor 488, Alexa Fluor 532, Alexa Fluor 546, Alexa Fluor 555, Alexa Fluor 568, Alexa Fluor 594, Alexa Fluor 647, Alexa Fluor 660, Alexa Fluor 680, Alexa Fluor 700, Alexa Fluor 750, and AlexaFluor790;

Dy677, Dy676, Dy682, Dy752, Dy780;

DyLight 350, DyLight 405, DyLight 488, DyLight 547, DyLight 550, DyLight 594, DyLight 633, DyLight 647, DyLight 650, DyLight 680, DyLight 755, and DyLight 800;

HiLyte Fluor 405, HiLyte Fluor 488, HiLyte Fluor 532, HiLyte Fluor 555, HiLyte™ Fluor 594, HiLyte Fluor 647, HiLyte Fluor 680, HiLyte Fluor 750;

IR800 (Dimethyl{4-[1,5,5-tris(4-dimethylaminophenyl)-2,4-pentadienylidene]-2,5-cyclohexadien-1-ylidene}ammonium perchlorate),

IRDye 650, IRDye 680RD, IRDye 680LT, IRDye 700, IRDye 700DX, IRDye 750, IRDye 800, IRDye 800CW, IRDye 800RS; and

ADS1065A, ADS1075A, ADS775MI, ADS775MP, ADS775PI, ADS775PP, ADS780HO, ADS780WS, ADS785WS, ADS790WS, ADS795WS, ADS798SM, ADS800AT, ADS815EI, ADS830AT, ADS830WS, ADS832WS, ADS845MC, and ADS920MC.

In more particular embodiments, the optical dye is selected from:

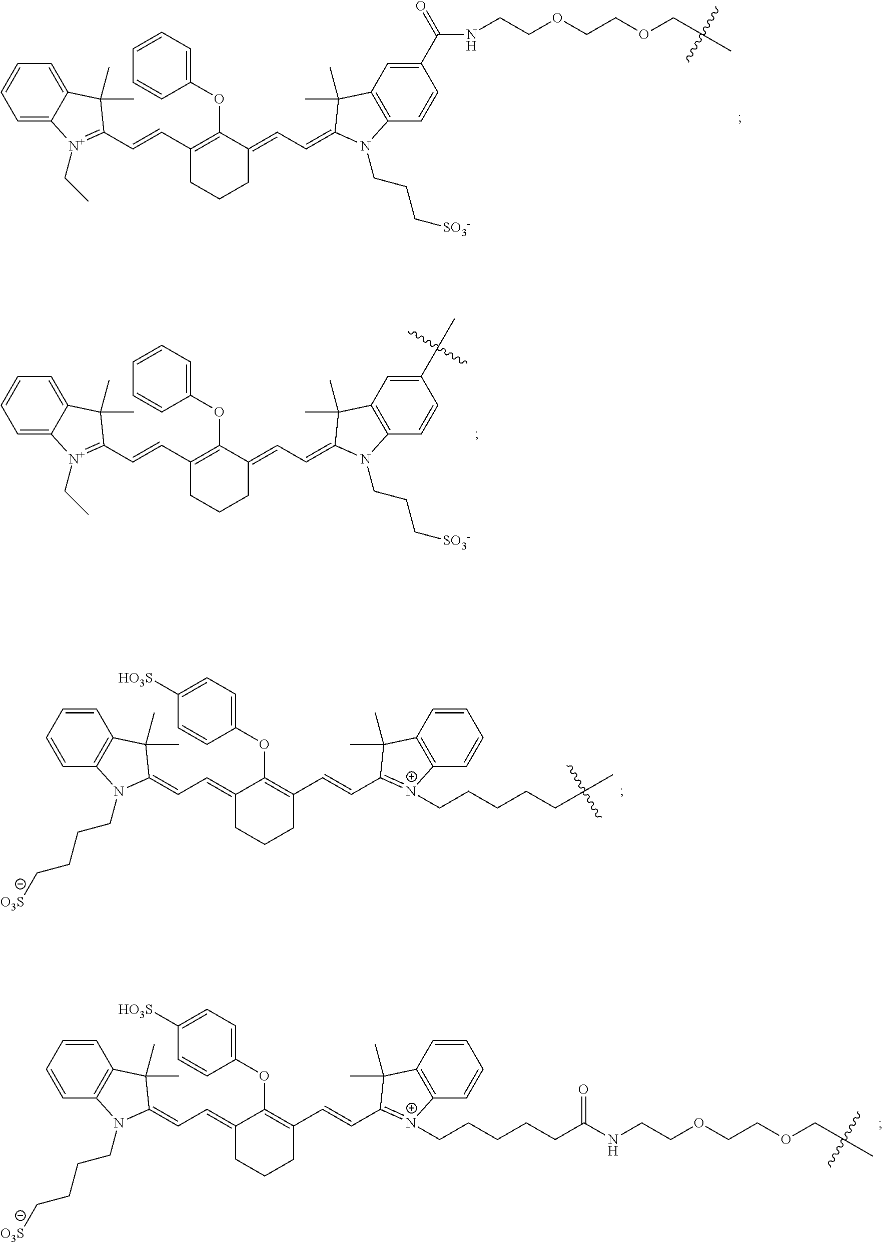

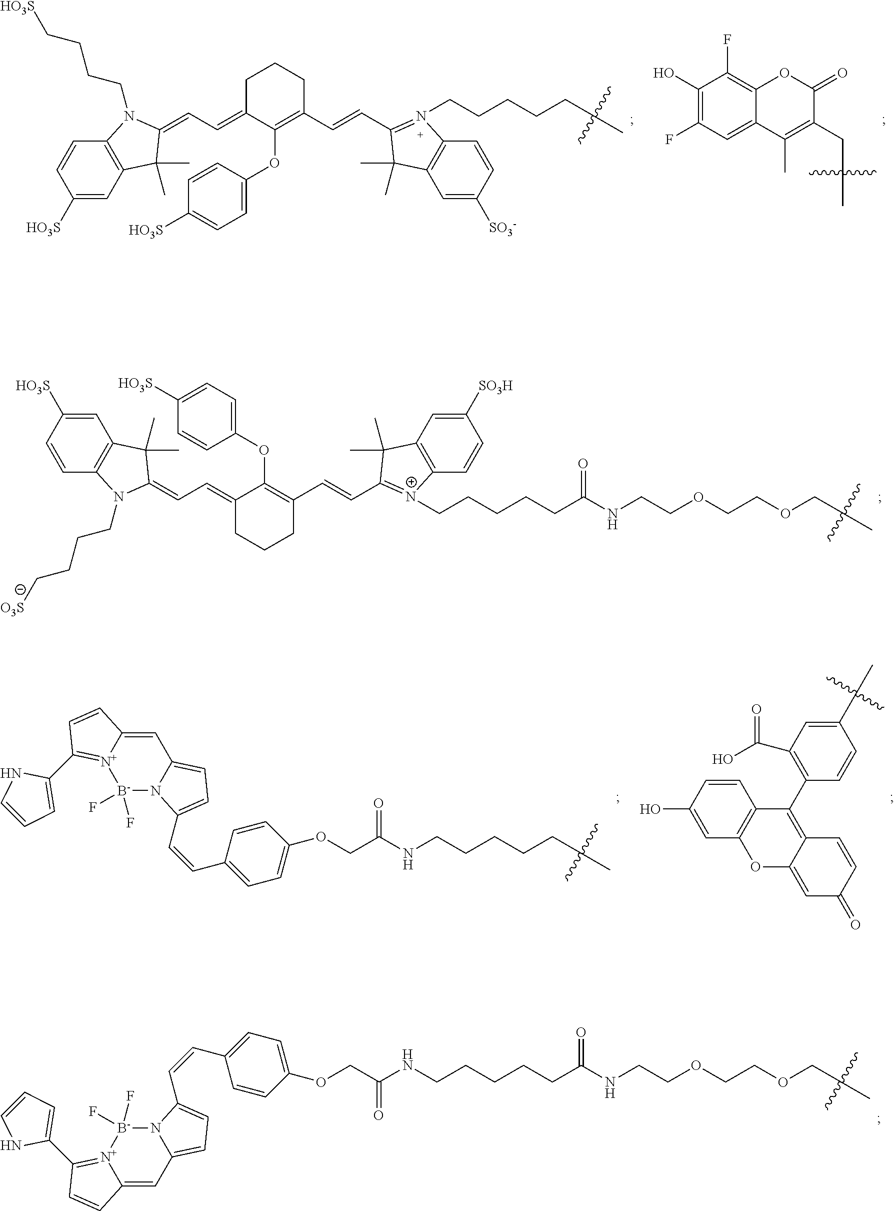

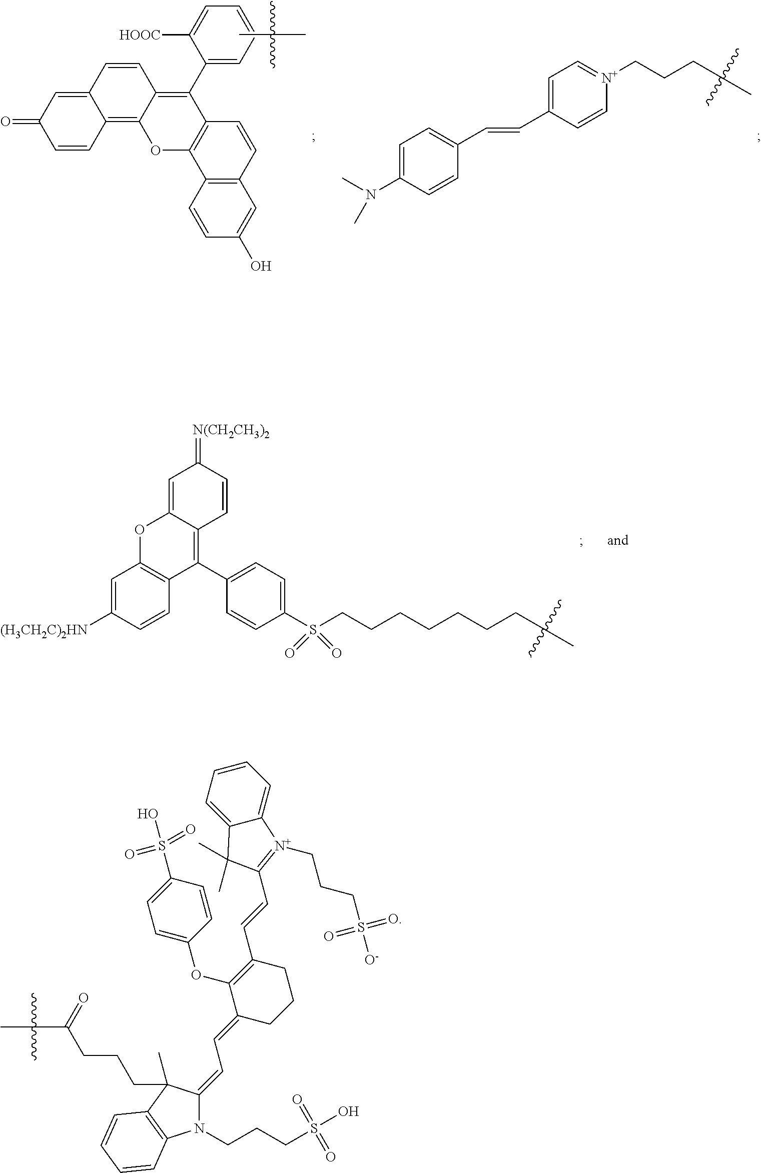

Suitable fluorsecent agents and linkers are disclosed in International PCT Patent Application No. WO2018232280 for PSMA Targeted Fluorescent Agents for Image Guided Surgery, to Pomper et al., published Dec. 20, 2018, which is incorporated herein by reference in its entirety.

In some embodiments, the presently disclosed compounds can be made by attaching near IR, closed chain, sulfo-cyanine dyes to prostate specific membrane antigen ligands via a linkage. For example, the prostate specific membrane antigen ligands used in the presently disclosed compounds can be synthesized as described in international PCT patent application publication no. WO 2010/108125, to Pomper et al., published Sep. 23, 2010, which is incorporated herein in its entirety.

In some embodiments, the presently disclosed compounds comprising a fluorescent dye and be used for photoacoustic imaging of tumors, including, but not limited to IRDye 800CW or IR820 (2-[2-[2-Chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt). See, for example, Zhang et al., Prostate-specific membrane antigen-targeted photoacoustic imaging of prostate cancer in vivo, J. of Biophotonics, 2018;11:e201800021.

In certain embodiments, the presently disclosed dendrimer can be used in photodynamic therapy when conjugated with a photosensitizers, such as porphyrins or Licor IRDye 700DX Dye, for example, see, “A PSMA-targeted theranostic agent for photodynamic therapy,” Chen Y, Chatterjee S, Lisok A, Minn I, Pullambhatla M, Wharram B, Wang Y, Jin J, Bhujwalla Z M, Nimmagadda S, Mease R C, and Pomper MG, J Photochem Photobiol B. 2017 167:111-116. Photodynamic therapy (PDT) is a minimally invasive cancer treatment and has been used in clinic to improve cancer patients' quality of life and survival time. The lack of specific delivery of the photosensitizers, however, is a significant limitation of PDT. Non-targeted, conventional photodynamic therapy cannot deliver the photosensitizers specifically to the tumor and the photosensitizers often circulate in the body long after treatment and cause sensitivity to light for several months.

Urea-based photosensitizers, which target prostate-specific membrane antigen (PSMA) for imaging and targeted therapy of PSMA-expressing tumors and cancers are disclosed in International PCT Patent Application No. WO2015057692 for Prostate-Specific Membrane Antigen-Targeted Photosensitizers for Photodynamic Therapy, to Pomper et al., published Apr. 23, 2015, and U.S. Pat. No. 10,232,058 for Prostate-Specific Membrane Antigen-Targeted Photosensitizers for Photodynamic Therapy, to Pomper et al., issued Mar. 19, 2019, each of which is incorporated by reference in their entirety.

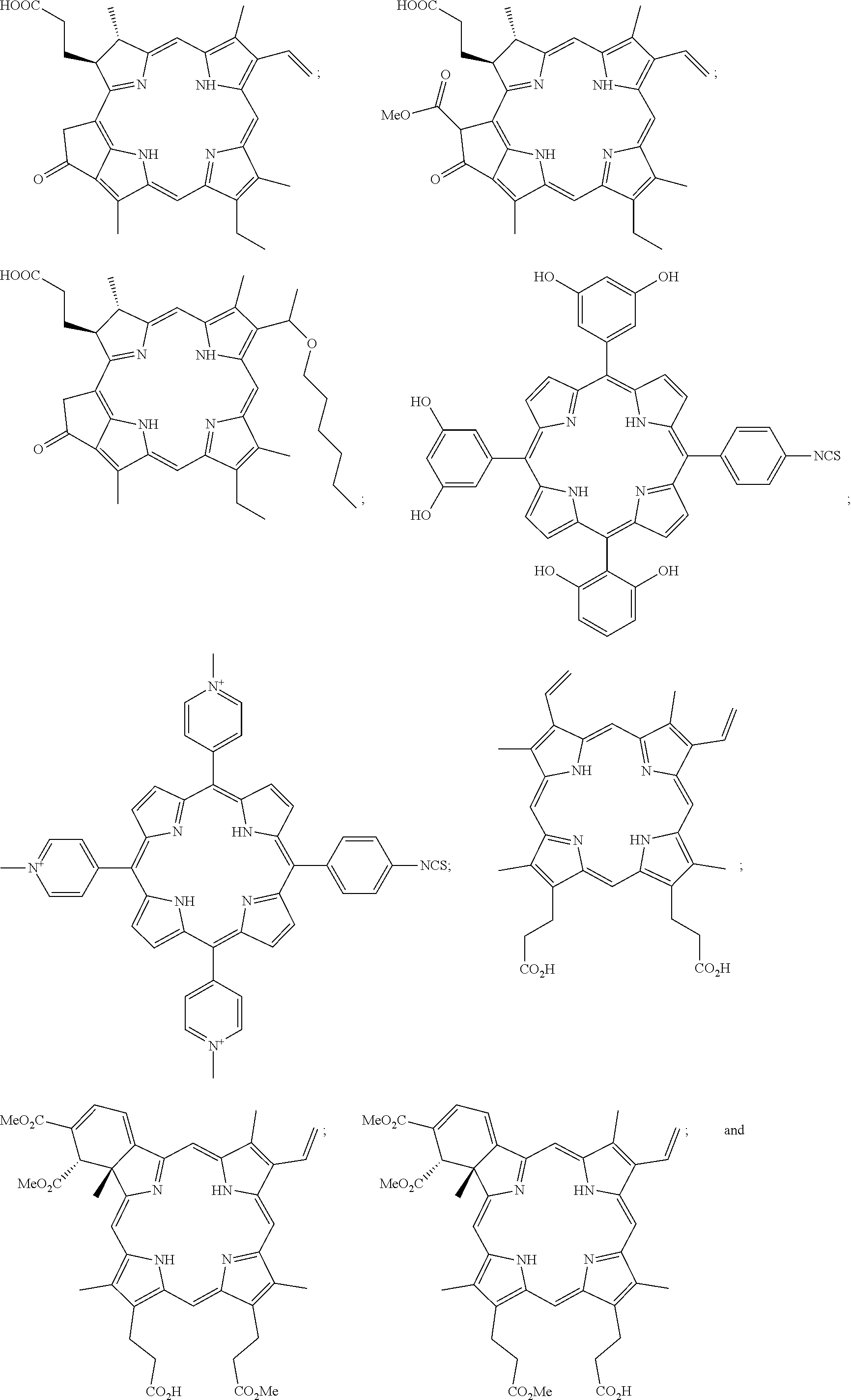

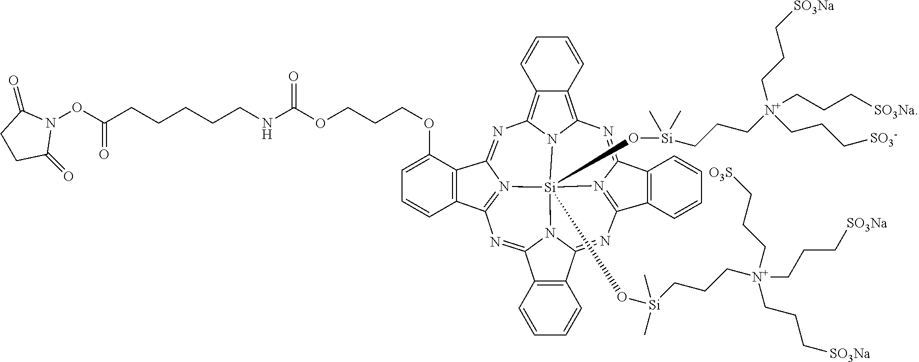

In some embodiments, the presently disclosed dendrimer can be conjugated to a chelating agent, which can bind a radiometal for imaging and/or radiotherapy, including endoradiotherapy. In more certain embodiments, the chelating agent is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid)), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid), (NOTAGA) 1,4,7-triazonane-1,4-diyl)diacetic acid, DFO (Deferoxamine), NETA ([4-[2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6] eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and BaBaSar.

In yet more certain embodiments, the chelating agent is selected from:

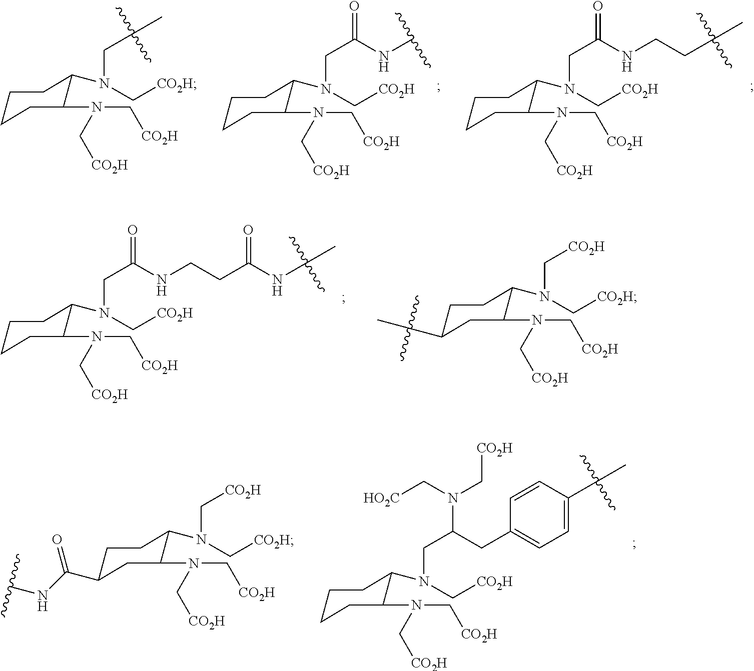

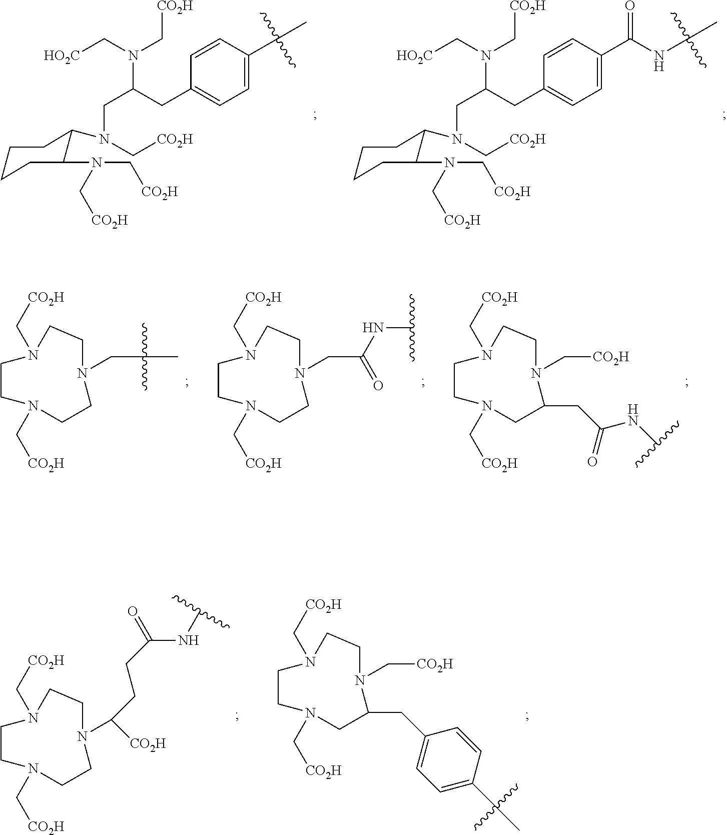

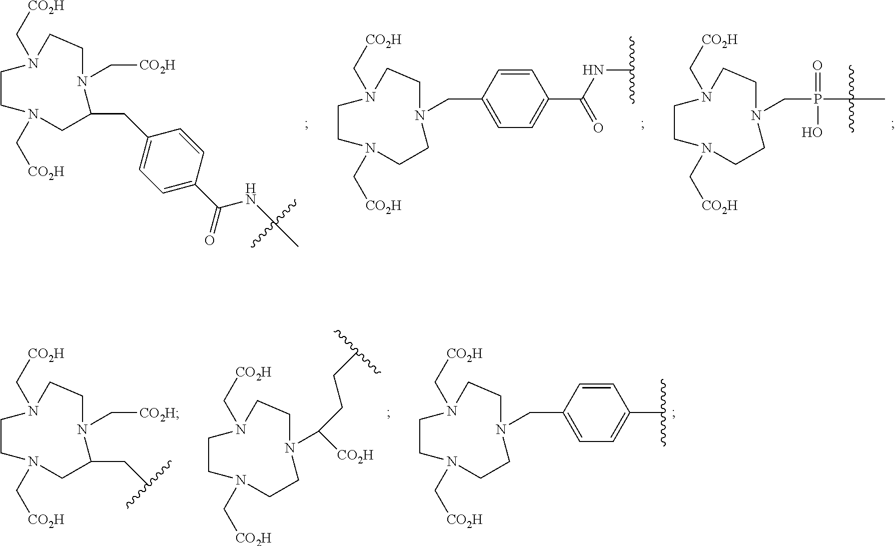 [SMILES: *NC(=O)c1ccc(CC2CN(CC(=O)O)CCN(CC(=O)O)CCN2CC(=O)O)cc1 ; *NC(=O)c1ccc(CN2CCN(CC(=O)O)CCN(CC(=O)O)CC2)cc1 ; *P(=O)(O)CN1CCN(CC(=O)O)CCN(CC(=O)O)CC1 ; *CC1CN(CC(=O)O)CCN(CC(=O)O)CCN1CC(=O)O ; *CCC(C(=O)O)N1CCN(CC(=O)O)CCN(CC(=O)O)CC1 ; *c1ccc(CN2CCN(CC(=O)O)CCN(CC(=O)O)CC2)cc1]

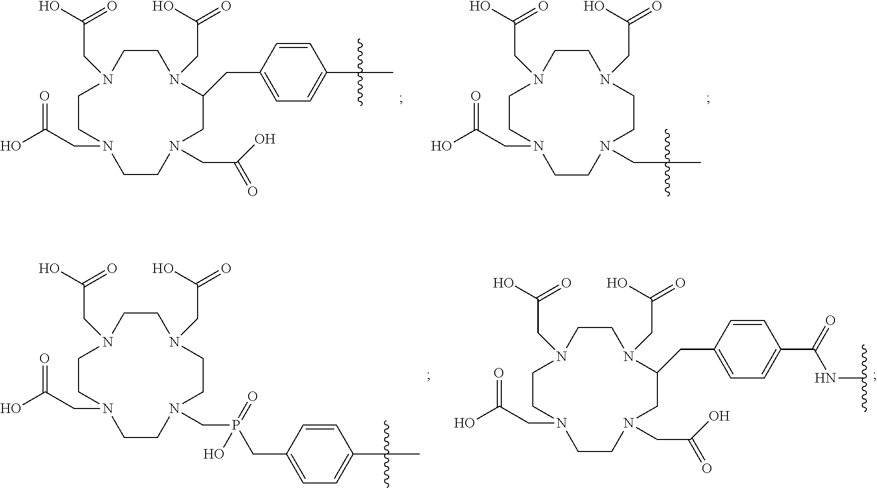 [SMILES: *c1ccc(CC2CN(CC(=O)O)CCN(CC(=O)O)CCN(CC(=O)O)CCN2CC(=O)O)cc1 ; *CN1CCN(CC(=O)O)CCN(CC(=O)O)CCN(CC(=O)O)CC1 ; *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1]

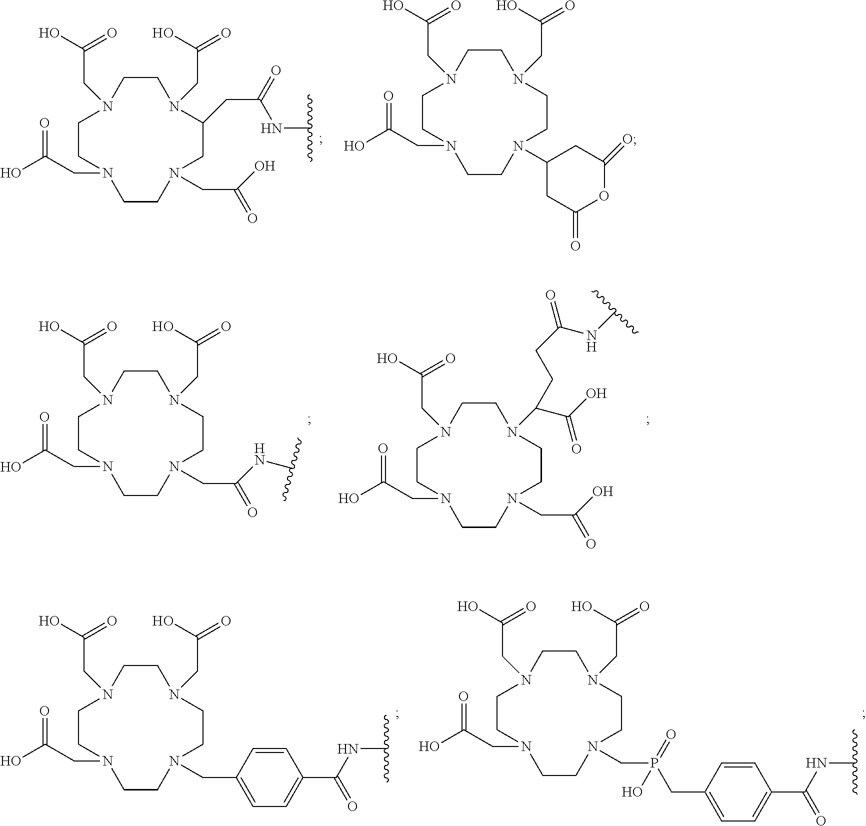 [SMILES: *NC(=O)CC1CN(CC(=O)O)CCN(CC(=O)O)CCN(CC(=O)O)CCN1CC(=O)O ; O=C(O)CN1CCN(CC(=O)O)CCN(C2CC(=O)OC(=O)C2)CCN(CC(=O)O)CC1 ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1 ; *NC(=O)c1ccc(CN2CCN(CC(=O)O)CCN(CC(=O)O)CCN(CC(=O)O)CC2)cc1 ; *NC(=O)c1ccc(CP(=O)(O)CN2CCN(CC(=O)O)CCN(CC(=O)O)CCN(CC(=O)O)CC2)cc1]

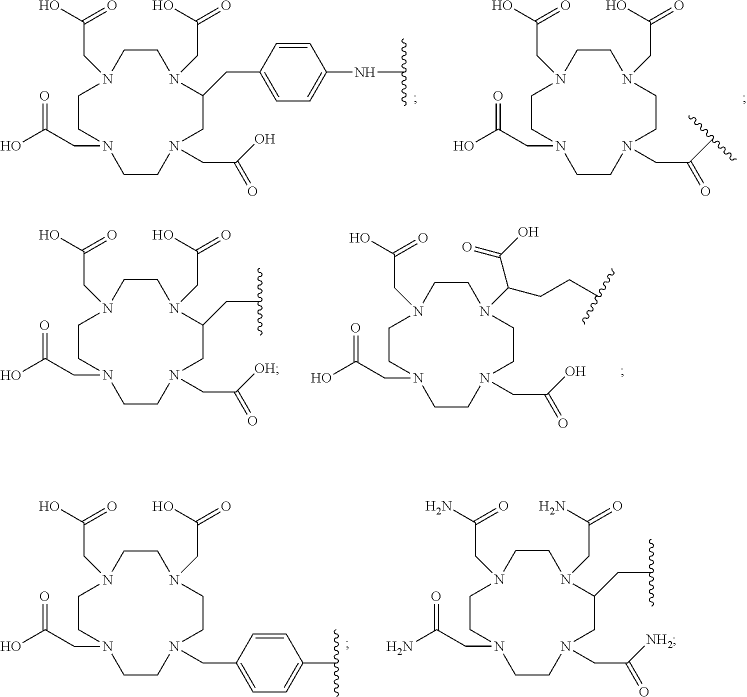 [SMILES: *Nc1ccc(CC2CN(CC(=O)O)CCN(CC(=O)O)CCN(CC(=O)O)CCN2CC(=O)O)cc1 ; *C(=O)CN1CCN(CC(=O)O)CCN(CC(=O)O)CCN(CC(=O)O)CC1 ; *CC1CN(CC(=O)O)CCN(CC(=O)O)CCN(CC(=O)O)CCN1CC(=O)O ; *CCC(C(=O)O)N1CCN(CC(=O)O)CCN(CC(=O)O)CCN(CC(=O)O)CC1 ; *c1ccc(CN2CCN(CC(=O)O)CCN(CC(=O)O)CCN(CC(=O)O)CC2)cc1 ; *CC1CN(CC(N)=O)CCN(CC(N)=O)CCN(CC(N)=O)CCN1CC(N)=O]

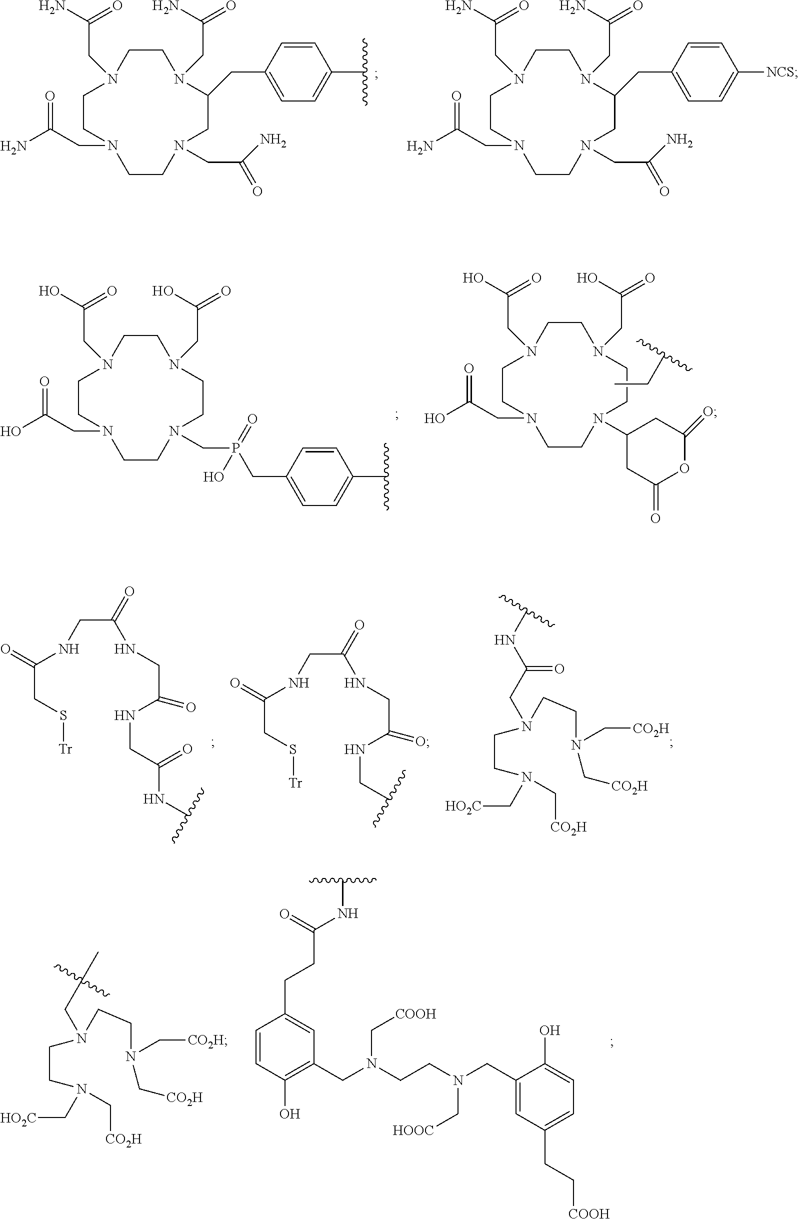

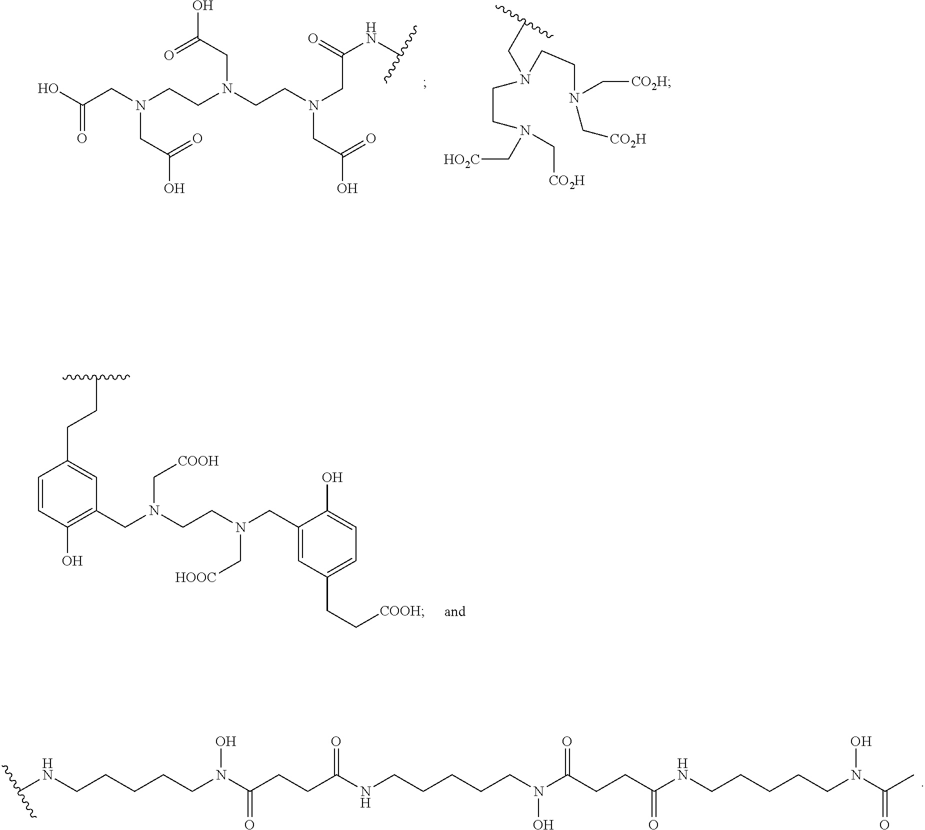 [SMILES: *NC(=O)CN(CCN(CCN(CC(=O)O)CC(=O)O)CC(=O)O)CC(=O)O ; *CN(CCN(CC(=O)O)CC(=O)O)CCN(CC(=O)O)CC(=O)O ; *CCc1ccc(O)c(CN(CCN(CC(=O)O)Cc2cc(CCC(=O)O)ccc2O)CC(=O)O)c1 ; *NCCCCCN(O)C(=O)CCC(=O)NCCCCCN(O)C(=O)CCC(=O)NCCCCCN(O)C(C)=O]

One of ordinary skilled in the art would appreciate that commercially-available chelating agents can include activating agents, for example, agents that can react with a primary amine. Such agents include, but are not limited to, N-hydroxysuccinimide (NHS), N-hydroxysulfosuccinimide (sulfo-NHS), anhydride, maleimide, N-benzyl, 4-isothiocyanatobenzyl (p-NCS-Bz), NH₂-MPAA, propargyl, TA, N-(2-aminoethyl)ethanamide, NH₂-PEG4, and hydrophilic dPEG spacer bound to a tetrafluorophenyl (TFP) ester. These agents can be bound to or form part of the linker which is bound to the chelating agent.

In certain embodiments, the chelating agent further comprises a radiometal. In particular embodiments, the radiometal is selected from ⁶⁰Cu, ⁶²Cu, ⁶⁴Cu, ⁶⁷Cu, ²⁰³Pb, ²¹²Pb, ²²⁵Ac, ¹⁷⁷Lu, ^{99m}Tc ⁶⁸Ga ¹⁴⁹Tb, ⁸⁶y ⁹⁰Y ¹¹¹In, ¹⁸⁶Re, ¹⁸⁸Re, ¹⁵³Sm, ⁸⁹Zr, ²¹³Bi, ²¹²Bi, ²¹²Pb, ⁶⁷Ga ⁴⁷Sc, Al¹⁸F, and ¹⁶⁶Ho.

In some embodiments, the presently disclosed dendrimers can be used as chelating agents, for example for forming gadolinium complexes suitable for use as magnetic resonance imaging (MRI) contrast agents.

The presently disclosed dendrimers also can encapsulate drugs due to their large void volume. For example, hydrophobic chemotherapeutic agents, such as docetaxel, can be encapsulated in the interior of the dendrimer. In other embodiments, anti-cancer agents, including, but not limited to, maytansine, and maytansinoids, including, but not limited to, ansamitocin, mertansine/emtansine (DM1), ravtansine/soravtansine (DM4), monomethyl auristatin e, methotrexate, doxorubicin, and paclitaxel. In particular embodiments, the therapeutic agent comprises mertansine (DM1). In some embodiments, a dye, can be substituted with one or more drugs via one or more cleavable bonds.

Representative uses of dendrimers for drug delivery are disclosed in “Nanoparticle Targeting of Anticancer Drug Improves Therapeutic Response in Animal Model of Human Epithelial Cancer,” Jolanta F. Kukowska-Latallo, Kimberly A. Candido,1 Zhengyi Cao, Shraddha S. Nigavekar, Istvan J. Majoros, Thommey P. Thomas, Lajos P. Balogh, Mohamed K. Khan, and James R. Baker, Jr., Cancer Res 2005; 65: (12). Jun. 15, 2005; “Potent Antitumor Activity of an Auristatin-Conjugated, Fully Human Monoclonal Antibody to Prostate-Specific Membrane Antigen,” Dangshe Ma, Christine E. Hopf, Andrew D. Malewicz, Gerald P. Donovan, Peter D. Senter, William F. Goeckeler, Paul J. Maddon, and William C. Olson, Clin Cancer Res 2006;12(8) Apr. 15, 2006; “PEGylated PAMAM dendrimere doxorubicin conjugate-hybridized gold nanorod for combined photothermal-chemotherapy,” Xiaojie Li, Munenobu Takashima, Eiji Yuba, Atsushi Harada, and Kenji Kono, Biomaterials 2014 35, 6576-6584; and “Targeting HER2-Positive Breast Cancer with Trastuzumab-DM1, an Antibody-Cytotoxic Drug Conjugate,” Gail D. Lewis Phillips, Guangmin Li, Debra L. Dugger, Lisa M. Crocker, Kathryn L. Parsons, Elaine Mai, Walter A. Blattler, John M. Lambert, Ravi V. J. Chari, Robert J. Lutz, Wai Lee T. Wong, Frederic S. Jacobson, Hartmut Koeppen, Ralph H. Schwall, Sara R. Kenkare-Mitra, Susan D. Spencer, and Mark X. Sliwkowski, Cancer Res 2008; 68:9280-9290, each of which is incorporated herein in their entirety.

In other embodiments, the presently disclosed dendrimers can encapsulate metallic clusters, such as gold or silver metallic clusters, to form composite nanoparticles that can be used for photothermal therapy, computerized tomography (CT) imaging, and the like. See, e.g., “Enhanced optical breakdown in KB cells labeled with folate-targeted silver-dendrimer composite nanodevices,” Christine Tse, Marwa J. Zohdy, Jing Yong Ye, Matthew O'Donnell, Wojciech Lesniak, and Lajos Balogh, Nanomedicine: Nanotechnology, Biology and Medicine, 2011 7 (1), Issue 1, 97-106; and “Targeted CT/MR dual mode imaging of tumors using multifunctional dendrimer-entrapped gold nanoparticles,” Qian Chen, Kangan Li, Shihui Wen, Hui Liu, Chen Peng, Hongdong Cai, Mingwu Shen, Guixiang Zhang, and Xiangyang Shia, Biomaterials 2013 34(21), 5200-5209, each of which is incorporated by reference in its entirety.

In other embodiments, the presently disclosed dendrimers also can be used as nanodevices. See, e.g., “Synthesis and Characterization of PAMAM Dendrimer-Based Multifunctional Nanodevices for Targeting avP3 Integrins,” Wojciech G. Lesniak, Muhammed S. T. Kariapper, Bindu M. Nair, Wei Tan, Alan Hutson, Lajos P. Balogh, and Mohamed K. Khan, Bioconjugate Chemistry 2007 18 (4), 1148-1154, each of which is incorporated by reference in its entirety.

B. Methods of Imaging and/or Treating a Cancer

In some embodiments, the presently disclosed subject matter provides a method for imaging and/or treating one or more PSMA-expressing expressing tumors or cells, the method comprising contacting the one or more PSMA expressing tumors or cells with an effective amount of a compound of formula (I) or formula (11), or a pharmaceutical formulation thereof and, if a method for imaging, taking an image.

In particular embodiments, the imaging is selected from optical imaging, photoacoustic imaging, and PET/CT imaging. In certain embodiments, the imaging is in vitro, in vivo, or ex vivo. In some embodiments, the method further comprises diagnosing and/or treating, based on the image, a disease or condition in a subject. In some embodiments, the method further comprises monitoring, based on the image, progression or regression of a disease or condition in a subject.

In certain embodiments, the method comprises imaging a cancer. In particular embodiments, the cancer is selected from prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature. In particular embodiments, the cancer comprises prostate cancer.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

In other embodiments, the method can be practiced in vitro or ex vivo by introducing, and preferably mixing, the compound and cell(s) or tumor(s) in a controlled environment, such as a culture dish or tube. The method can be practiced in vivo, in which case contacting means exposing the target in a subject to at least one compound of the presently disclosed subject matter, such as administering the compound to a subject via any suitable route. According to the presently disclosed subject matter, contacting may comprise introducing, exposing, and the like, the compound at a site distant to the cells to be contacted, and allowing the bodily functions of the subject, or natural (e.g., diffusion) or man-induced (e.g., swirling) movements of fluids to result in contact of the compound and the target.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. In some embodiments, the subject is human. In other embodiments, the subject is non-human.

### C. Pharmaceutical Compositions and Administration

The present disclosure provides a pharmaceutical composition including one compound of formula (I) or formula (II) alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (₂0′ ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (₂0th ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (₂0′ ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions, and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### D. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O—is equivalent to —OCH₂—; —C(═O)O—is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁₋₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl, or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The term “heteroalkyl,” by itself or in combination with another term, means, unless otherwise stated, a stable straight or branched chain having from 1 to 20 carbon atoms or heteroatoms or a cyclic hydrocarbon group having from 3 to 10 carbon atoms or heteroatoms, or combinations thereof, consisting of at least one carbon atom and at least one heteroatom selected from O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkylene moiety, also as defined above, e.g., a C₁₋₂₀ alkylene moiety. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from 0, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidinyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated hydrocarbon has one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C2-20 inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₂₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched, or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)-(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′—represents both —C(O)OR′—and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). The term “haloaryl,” however, as used herein is meant to cover only aryls substituted with one or more halogens.

Where a heteroalkyl, heterocycloalkyl, or heteroaryl includes a specific number of members (e.g., “3 to 7 membered”), the term “member” refers to a carbon or heteroatom.

Further, a structure represented generally by the formula:

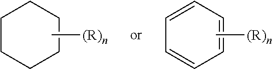

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

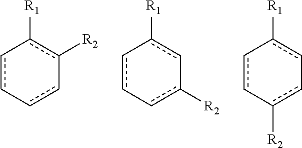

and the like.

A dashed line representing a bond in a cyclic ring structure indicates that the bond can be either present or absent in the ring. That is, a dashed line representing a bond in a cyclic ring structure indicates that the ring structure is selected from a saturated ring structure, a partially saturated ring structure, and an unsaturated ring structure.

The symbol [symbol] denotes the point of attachment of a moiety to the remainder of the molecule.

[symbol]

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, =NR′, =N—OR′, —NR′R″, —SR′, —halogen, —SiR′R″R″′, —OC(O)R′, —C(O)R′, —CO₂R′,—C(O)NR′R″, —OC(O)NR′R″, —NR″C(O)R′, —NR′—C(O)NR″R″′, —NR″C(O)OR′, —NR—C(NR′R″)=NR″′, —S(O)R′, —S(O)₂R′, —S(O)₂NR′R″, —NRSO₂R′, —CN, CF₃, fluorinated C₁₋₄ alkyl, and —NO₂ in a number ranging from zero to (2m′+1), where m′ is the total number of carbon atoms in such groups. R′, R″, R″′ and R″′ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R″′ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R″′, —OC(O)R′, —C(O)R′, —CO₂R′, —C(O)NR′R″, —OC(O)NR′R″, —NR″C(O)R′, —NR′—C(O)NR″R″′, —NR″C(O)OR′, —NR—C(NR′R″R′″)=NR″″, —NR—C(NR′R″)=NR″′—S(O)R′, —S(O)₂R′, —S(O)₂NR′R″, —NRSO₂R′, —CN and —NO₂, —R′, —N₃, —CH(Ph)₂, fluoro(C₁₋₄)alkoxo, and fluoro(C₁₋₄)alkyl, in a number ranging from zero to the total number of open valences on aromatic ring system; and where R′, R″, R″′ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R″′ and R″″ groups when more than one of these groups is present.

Two of the substituents on adjacent atoms of aryl or heteroaryl ring may optionally form a ring of the formula —T—C(O)-(CRR′)_{q}—U—, wherein T and U are independently —NR—, —O—, —CRR′—or a single bond, and q is an integer of from 0 to 3. Alternatively, two of the substituents on adjacent atoms of aryl or heteroaryl ring may optionally be replaced with a substituent of the formula —A—(CH₂)ᵣ—B—, wherein A and B are independently —CRR′—, —O—, —NR—, —S—, —S(O)—, —S(O)₂—, —S(O)₂NR′—or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R″′)ₐ—, where s and d are independently integers of from 0 to 3, and X′ is —O—, —NR′—, —S—, —S(O)—, —S(O)₂—, or —S(O)₂NR′—. The substituents R, R′, R″ and R″′ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)—and a 2-phenylacetyl group.

Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl—O— and alkynyl—O—) group attached to the parent molecular moiety through an oxygen atom, wherein the terms “alkyl,” “alkenyl,” and “alkynyl” are as previously described and can include C₁-20 inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)—group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)—group. Exemplary aryloxycarbonyl groups include phenoxy- and naphthoxy-carbonyl.

“Aralkoxycarbonyl” refers to an aralkyl-O—C(═O)—group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR. “Acyloxyl” refers to an acyl-O— group wherein acyl is as previously described. The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups, respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom.

The term alkylamino refers to a group having the structure —NHR′ wherein R′ is an alkyl group, as previously defined; whereas the term dialkylamino refers to a group having the structure —NR′R″, wherein R′ and R″ are each independently selected from alkyl groups. The term trialkylamino refers to a group having the structure —NR′R″R″′, wherein R′, R″, and R′ are each independently selected from alkyl groups. Additionally, R′, R″, and/or R′ taken together may optionally be —(CH₂)k-where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S—and alkynyl-S—) group attached to the parent molecular moiety through a sulfur atom. Examples of thioalkoxyl moieties include, but are not limited to, methylthio, ethylthio, propylthio, isopropylthio, n-butylthio, and the like. “Acylamino” refers to an acyl-NH—group wherein acyl is as previously described. “Aroylamino” refers to an aroyl-NH—group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The term “cyano” refers to the —C—N group.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁₋₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)—or (S)—or, as D- or L-for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)—and (S)—, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms.

For example, compounds having the present structures with the replacement of a hydrogen by a deuterium or tritium, or the replacement of a carbon by ¹³C—or ¹³ C—enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates, and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)— catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

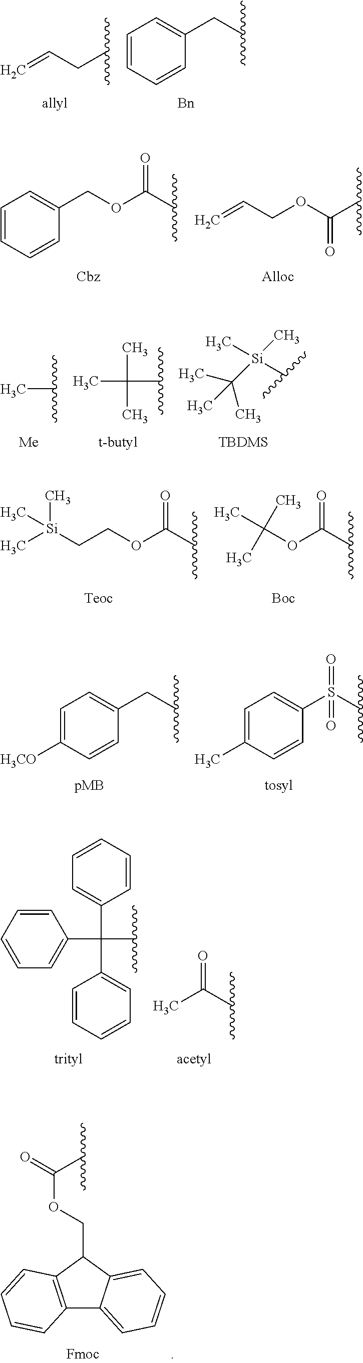

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments 30 1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### 1.1 Overview

The prostate-specific membrane antigen (PSMA) is a viable target for detection and management of prostate cancer (PC). Detection of PSMA expression has an important utility in diagnosis of PC and the active surveillance and monitoring of a therapeutic response. PSMA has been leveraged for radiopharmaceutical therapy and targeted drug delivery by antibody-drug conjugates and polylactic acid-polyethylene glycol (PLA-PEG)-based nanoparticles.

Poly(amidoamine) [PAMAM] dendrimers serve as versatile scaffolds for imaging agents and drug delivery that can be tailored to different sizes and compositions depending on the application. The presently disclosed subject matter provides generation-4 PAMAM dendrimers to develop PSMA-targeted contrast agents for optical and photoacoustic imaging (PA). The Lys-Glu-urea low molecular weight inhibitor is used as a targeting moiety, as it was developed and reported previously to facilitate highly specific in vivo targeting of PSMA in PC tumor. To enable near infrared fluorescence (NIR FL) and PA imaging, the dendrimer was conjugated with a different number of sulfo-cyanine7.5 (Cy7.5) NIR red dyes (2, 4, 6 and 8), and the remaining terminal primary amines were capped with butane-1,2-diol.

In other embodiments, the presently disclosed subject matter also includes a novel low-molecular-weight (LMW) agent composed of Cy7.5-lysine-suberic acid-lysine urea-glutamic acid (Cy7.5-K-SA-KEU).

The presently disclosed PSMA-targeted contrast agents exhibited high in vitro target specificity and preferential accumulation in PSMA⁺ PC3 PIP xenografts vs. isogenic PSMA-PC3 flu tumors that allowed for optical and PA imaging of PSMA expression. The results indicate that the presently disclosed contrast agents have a great potential for clinical application in the minimally invasive detection of PSMA expression in patients with PC by PA for diagnosis and the active surveillance and monitoring of therapeutic responses and intra-operative fluorescence surgical guidance for visualization of positive surgical margins that are associated with tumor recurrences and may result in debilitating additional therapies.

### 1.2 Scope

This disclosure describes the synthesis of LMW and generation-4 PAMAM dendrimer-based PSMA-targeted contrast agents for PA and optical imaging and their evaluation in vitro, as well as in vivo using an experimental model of PC, and the optimization of the spectroscopic PA imaging system. LMW Cy7.5-K-SA-K-KEU was synthetized as the previous imaging agent YC-27, but IRDye800CW NIR dye was substituted with a Cy7.5 NIR dye providing improved PA contrast compared to YC-27. Four PSMA-targeted conjugates with different number of Cy7.5 dyes (ranging from 2 to 8) and one control non-targeted dendrimer conjugate equipped with six Cy7.5 dyes were formulated using a facile one-pot synthesis.

Cy7.5-K-SA-K-KEU and all five PSMA-targeted dendrimer conjugates exhibit superior in vitro target specificity with dissociation constant ranging from 0.078 to 0.78 μM. Conjugate I—Cy7.5-K-SA-K-KEU, conjugate IV—generation-4 PAMAM dendrimer conjugated with six sulfo-Cy7.5 dyes, nine SA-KEU targeting agents and capped with fifty-three butane-1,2-diol moieties and conjugate VI (non-targeted control dendrimer)—generation 4 PAMAM dendrimer conjugated with six Cy7.5 dyes and capped with fifty-two butane-1,2-diol moieties were selected for in vivo evaluation.

Conjugate I and IV showed preferential accumulation in PSMA⁺ PC3 PIP xenografts vs. isogenic PSMA-PC3 flu tumors, which provided florescence and PA signal 5 hrs after administration, which persisted up to 3 days. Conjugate IV showed lower signal intensity in PSMA⁺ PC3 PIP tumors than conjugate I in NIR FL imaging with superior spectral decomposition contrast, indicating significantly better sensitivity for detection of PSMA expression with PA imaging. The advantage of the presently disclosed imaging agents as compared to anti-PSMA antibody conjugates or other relatively large nanoparticles with size of about 50 nm to 100 nm used for imaging PSMA, is their straightforward formulation, low off-target tissue accumulation, highly preferential and persistent accumulation PSMA positive tumors throughout their entire volume that provide strong potential for clinical translation with multitude of applications. In addition, the spectroscopic PA imaging system was optimized to increase the imaging speed and the sensitivity and specificity of PC detection.

### 1.3 Background

Molecular and functional imaging has proven to provide another layer of precision in the diagnosis, prognosis, treatment planning and therapeutic response assessment of PC. Picchio et al., 2015. To this end, noninvasive imaging of prostate-specific membrane antigen (PSMA) expression is being extensively evaluated, as it is overexpressed in most PCs, compared to normal prostate tissue and has been associated rapid progression of the disease and poor prognosis. Kiess et al., 2015; Maurer et al., 2016. PSMA expression in cancers has been imaged with low molecular weight (LMW) agents, nanoparticles and monoclonal antibodies using a different type of imaging modalities including nuclear, magnetic resonance and optical imaging. Kiess et al., 2015; Maurer et al., 2016; Azad et al., 2015; Chen et al., 2009; Dogra et al., 2016.

It was recently demonstrated that PSMA expression also can be assessed in vivo with photoacoustic imaging and commercially available YC-27 near-infrared optical imaging agent, composed of lysine-subaric acid linker (for short K-SA), lysine-glutamate-urea (for short KEU) PSMA inhibitor and IRDye800CW NIR fluorescent dye, which might be leveraged to a novel noninvasive active surveillance of PC using noninvasive PA imaging. Zhang et al., 2018. PA imaging is a quickly emerging biomedical noninvasive hybrid imaging modality that combines the superior contrast and specificity of optical imaging with sub-micrometers spatial resolution of ultrasound (US) imaging. Cheng et al., 2017; Fu et al., 2019; Wang et al., 2016.

PA imaging has ability to detect hemoglobin, lipids, water and other light-absorbing chromophores providing better specificity and penetration depth than conventional US and optical imaging, respectively, providing functional and molecular imaging capabilities for a wide range of applications in both clinics and pre-clinical studies. To date, a number of contrast agents for PA imaging have been developed, including gold nanocrystals, graphene-based agents, carbon nanotubes, organic nanoparticles, and semiconducting polymeric nanoparticles, which are extensively evaluated in terms of their application in wide range of biomedical imaging. Fu et al., 2019.

An ideal PA contrast agent should exhibit low quantum yield, high molar-extinction coefficient, NIR absorption window, photostability, biocompatibility, as well as high target specificity. All these features might be assembled using poly(amidoamine) [PAMAM] dendrimers having high tunable properties. PAMAM dendrimers are well-defined scaffolds with low polydispersity, large number of reactive terminal groups, large interior void volume, flexibility, biocompatibility and straightforward modification that garnered development of nanomaterials for various biomedical applications including targeted-drug delivery, transfection and biomedical imaging. de Araujo et al., 2018.

It has been recently demonstrated that generation-4 PAMAM dendrimers conjugated with KEU show superior uptake PSMA⁺ PC3 PIP tumors vs. PSMA-PC3 flu tumors with low uptake in off-target tissues. Lesniak et al., 2019. The pharmacokinetics of these dendrimers indicate that they may be used for targeting PSMA-expressing tissues with imaging and therapeutic agents. The presently disclosed subject matter presents a simulation-aided framework for spectroscopic PA imaging of a PSMA-targeted agent to optimize the sensitivity, specificity, and temporal resolution of aggressive PC detection. In addition, the development of PSMA-targeted dendrimers for optical and photoacoustic dual imaging contrast agents and their evaluation in an experimental model of PC is shown.

### 1.4 Synthesis

Di-tert-butyl (((S)-1-(tert-butoxy)-6-(8-((2,5-dioxopyrrolidin-1-yl)oxy)-8-oxooctanamido)-loxohexan-2-yl) carbamoyl)-L-glutamate (1): First, protected Glu-Urea-Lys (KEU) was synthetized as previously reported. Maresca et al., 2009. To the stirred solution of dimethylformamide (7 mL) containing disuccinimidyl suberate (DSS, 415 mg, 1.12 mmol, 2.2 mol equivalent) 3 mL of dimethylformamide (DMF) containing protected KEU (250 mg, 0.513 mmol, 1.0 mol equivalent) and triethylamine (71 μL, 0.513 mmol, 1.0 mol equivalent) was added dropwise for 20 min under nitrogen atmosphere and at room temperature (RT). The resulting mixture was stirred for additional 2 h at RT, concentrated under reduced pressure at 40° C. Crude product was purified by a silica column chromatography using acetonitrile and dichloromethane as eluents (40% acetonitrile in dichloromethane) to afford compound 1 as a viscous liquid (228 mg, yield 60%), ¹H NMR (CDCl₃): 6.30 (t, J=5.5 Hz, 1H), 5.45 (dd, J=8.0, 18.5 Hz, 2H), 4.35 (m, 2H), 3.15-3.35 (m, 2H), 2.82 (s, 4H), 2.65 (t, J=7.0 Hz, 2H), 2.41 (m, 2H), 2.20 (t, J=7.0 Hz, 2H), 2.18-1.21 (m, 43H); ESI-MS m/z: 741.4 [M+H]. Then 200 mg (0.27 mmol) of compound 1 was dissolved in the solution composed of 2 mL of trifluoroacetic acid (TFA) and 2 mL of dichloromethane (DCM) and resulted mixture was stirred at RT for 2 h, followed by drying in vacuo. The crude product was purified by reverse phase flash chromatography using 0.1% TFA in H₂O (A) and 0.1% TFA in acetonitrile (B) as eluents followed by lyophilization, which afforded NHS-SA-KEU in the yield of 90% (139 mg). Flash chromatography purification was carried out using a Biotage Isolera One system with detection wavelength set to 220 nm, Biotage SNAP Ultra C18 column (12 g) and gradient elution starting with 90% H₂O (0.1% TFA) and 10% ACN (0.1% TFA), reaching 90% of ACN. Product eluted at 50% (β) to 55% (A) fraction. ¹H-NMR (500 MHz, CD₃OD): δ4.33-4.23 (m, 2H), 3.17 (t, J=7.0 Hz, 2H), 2.83 (s, 4H), 2.62 (t, J=7.5 Hz, 2H), 2.46-2.33 (m, 2H), 2.21-2.09 (m, 3H), 1.92-1.59 (m, 7H), 1.55-1.33 (m, 8H); ESI-MS m/z: 573.2 [M+H].
(3S,7S,22S)-26-Amino-5,13,20-trioxo-4,6,12,21-tetraazahexacosane-1,3,7,22-tetracarboxylic acid (2): 70 mg of compound 1 (0.094 mmol, 1.0 mol equivalent) and 27.9 mg of N′—Boc-L-Lysine (0.113 mmol, 1.2 mol equivalent) were dissolved in 500 μL of dimethyl sulfoxide (DMSO) and 49 μL of diisopropylethylamine (DIPEA, 0.283 mmol, 3.0 mol equivalent) was added. The resulting mixture was stirred for 2 h at RT and concentrated in vacuo to afford thick residue. This residue was dissolved in solution containing 2 mL of TFA and 2 mL of DCM and stirred for 2 h at RT, followed by concentrated in vacuo. The crude product was purified by reverse phase flash chromatography using 0.10% TFA in H₂O (A) and 0.10% TFA in acetonitrile (B) as eluents followed by lyophilization, which provided K-SA-KEU as a colorless solid in the yield of 80% (45 mg). Flash chromatography purification was achieved using Biotage Isolera One system, X 220 nm, Biotage SNAP Ultra C18 column (12 g), solvent gradient: 90% H₂O (0.1% TFA) and 10% ACN (0.1% TFA), reaching 90% of ACN, product eluted at 40% to 45% of (β) in (A) fraction]. ¹H-NMR (500 MHz, CD₃OD): δ4.44-4.37 (m, 1H), 4.34-4.22 (m, 2H), 3.17 (t, J=5.5 Hz, 2H), 2.95-2.88 (m, 2H), 2.45-2.37 (m, 2H), 2.25 (t, J=7.0 Hz, 2H), 2.17 (t, J=7.5 Hz, 2H), 1.97-1.79 (m, 4H), 1.77-1.26 (m, 18H); ESI-MS m/z: 604.3 [M+H].
1,1-Dimethyl-3-((3S,7S,22S)-1,3,7,22-tetracarboxy-5,13,20,28-tetraoxo-4,6,12,21,27-pentaazatritriacontan-33-yl)-2-((E)-2-((E)-3-((E)-2-(1,1,3-trimethyl-6,8-disulfonato-1,3-dihydro-2H-benzo[efindol-2-ylidene)ethylidene)cyclohex-1-en-1-yl)vinyl)-1H-benzoindol-3-ium-6,8-disulfonate (Conjugate I—Cy7.5-K-SA-KEU): To a stirred solution of DMSO (20 μL) containing K-SA-KEU (3.83 mg, 63 μmol, 1.5 mol equivalent) and sulfo-Cy7.5-NHS ester (5 mg, 42 μmol, 1.0 mol equivalent) 8.8 μL of DIPEA (50 μmol, 12 mol equivalent) was added. This reaction mixture was stirred for 2 h at RT and concentrated in vacuo, which afforded a thick residue. The crude product was purified using a Varian ProStar HPLC pumps connected with a semi-preparative C-18 Luna column (5 μm, 10×250 mm, Phenomenex) and an Agilent 1260 Infinity photodiode array detector. A gradient elution starting with 98% of aqueous 20 mM triethyl ammonium acetate buffer (A) and 2% acetonitrile (B), reaching 100% of B in 30 min at flow rate of 4 ml/min was applied. The product was collected between 11.5 and 12.5 min of elution, followed by evaporation on a rotary evaporator and lyophilization to afford Cy7.5-K-SA-KEU conjugate I as a dark green solid (1.8 mg, yield 70%).
1.5 Synthesis of dendrimer conjugates. All dendrimer conjugates were synthesized using one-pot approach as recently reported. Lesniak et al., 2019.

In atypical reaction 10 mg of G4(NH₂)₆₄ (0.7 μmol) dissolved in 2 mL of PBS mixed with 100 μL of DMSO containing 1.66 mg (1.41 μmol, for conjugated II) or 3.721 mg (3.15 μmol for conjugate III) or 5.81 mg (4.92 μmol, for conjugate IV) or 10 mg (8.44 μmol, for conjugate V) of Cy7.5-NHS. After 2 h of stirring at RT an aliquot of each reaction mixture was subjected to MALDI-TOF mass spectrometry to assess number of Cy7.5 dyes conjugated with dendrimer. Next, 5 mg (9.58 μmol) of NHS-SA-KEU dissolved in 100 μL of DMSO was added to each reaction mixture and reacted for 2 h, followed by MADLI-TOF mass spectrometric analysis to confirm covalent attachment of SA-KEU with G4(NH₂)₆₂(Cy7.5)2, G4(NH₂)₅₈(Cy7.5)6, G4(NH₂)₆(Cy7.5)4 and G4(NH₂)₅₆(Cy7.5)ₛ conjugates. In the final synthetic step, 0.2 mL (3 mmol) of glycidol was added to each reaction mixture, which were stirred overnight at RT to cap the remaining unmodified primary amines with butane-1,2-diol. The non-targeted control dendrimer was formulated by reacting G4(NH₂)₆₄ dendrimer with 5.81 mg (4.92 μmol) of Cy7.5 dye and 0.2 mL (3 mmol) of glycidol. Resulting conjugates II [G4(NH₂)₂₀(Cy7.5)₂(SA-KEU)₁₀(Bdiol)32], III [G4(NH₂)(Cy7.5)4(SA-KEU)₉(Bdiol)30], IV [G4(Cy7.5)6(SA-KEU)₉(Bdiol)53] and V [G4(Cy7.5)ₛ(SA-KEU)12(Bdiol)52] and VI [G4(NH₂)₁₃(Cy7.5)6(Bdiol)45] were purified using Amicon centrifugal filters with 10,000 Da MWCO (Millipore Sigma, Rockville Md.) and deionized water, followed by lyophilization and characterization by RP-HPLC, MALDI-TOF MS, UV-Vis and fluorescence spectroscopy.

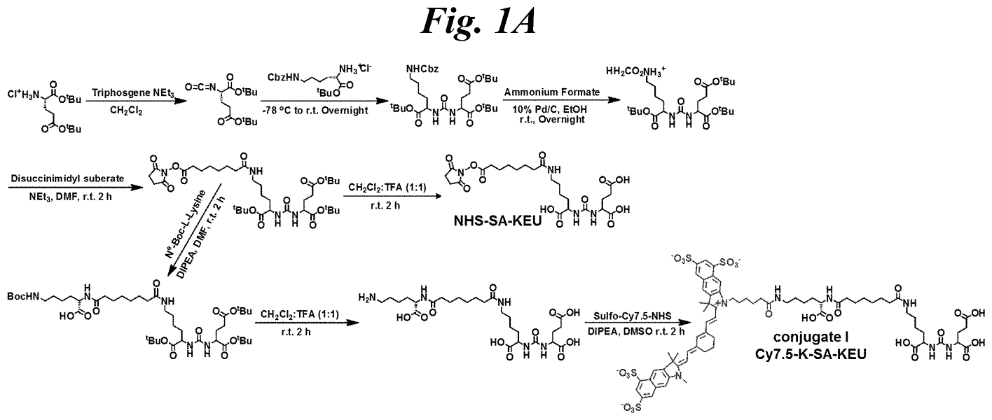

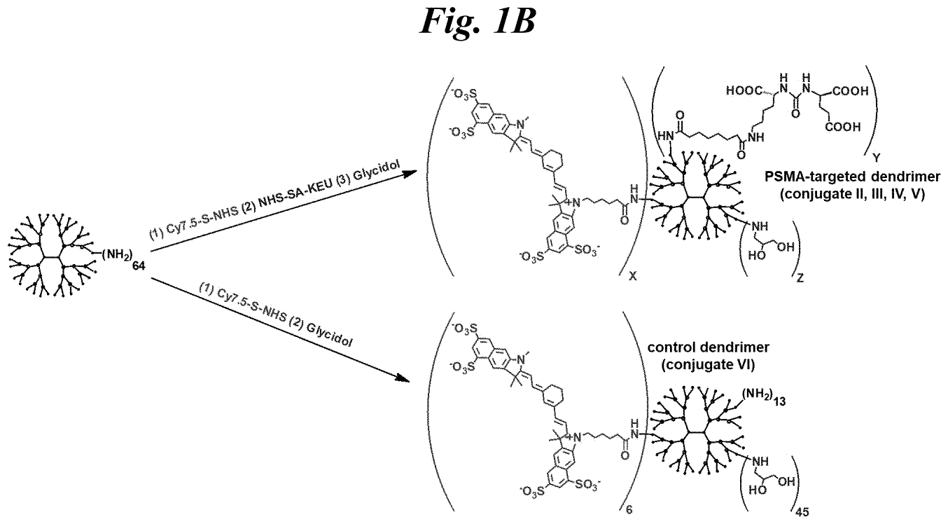

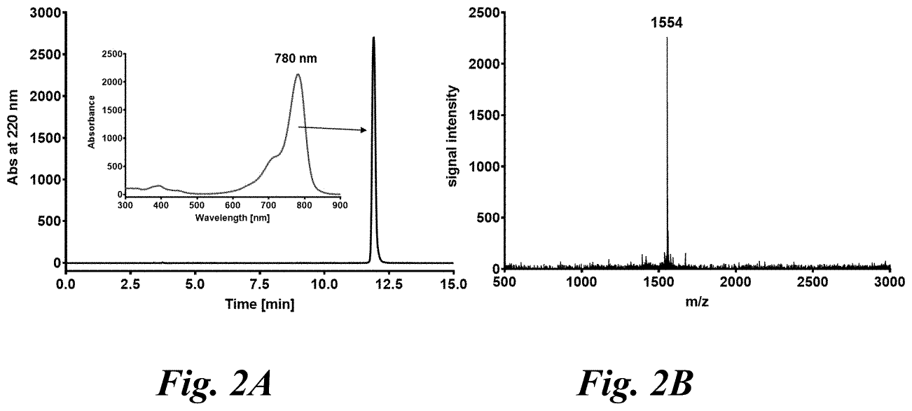

Synthesis of the presently disclosed PSMA-targeted contrast agents is presented in FIG. 1. A well-established LMW Lys-Glu-urea (KEU) inhibitor with picomolar affinity to PSMA, which has been extensively studied, was selected as a targeting moiety. KEU was utilized to synthetize LMW sulfo-Cy7.5-K-SA-KEU contrast agent with single Cy7.5 NIR-dye and NHS-SA-KEU primary amine reactive moiety for conjugation with dendrimers. Cy7.5-K-SA-K-KEU was synthetized as reported for the imaging agent YC-27, Maresca et al., 2009, but IRDye800CW near infrared (NIR) dye was substituted with Cy7.5 NIR dye. To confirm its identity and purity, Cy7.5-K-SA-K-KEU was analyzed using RP-HPLC and MALDI-TOF mass spectrometry (FIG. 2). In case of dendrimer conjugates, to avoid off-target uptake (mainly liver and spleen uptake) and achieve preferential renal clearance of PSMA-targeted dendrimers upon intravenous administration, generation 4 amine-terminated PAMAM dendrimer with an approximately 4-nm hydrodynamic radius was selected as the starting material.

The synthesis of NHS-SA-KEU low molecular weight PSMA targeting moiety for conjugation with dendrimer and synthesis of Cy7.5-K-SA-KEU imaging agent is shown in FIG. 1A. Initially, protected Glu-Urea-Lys (KEU) was synthetized as previously reported. Maresca et al., 2009.

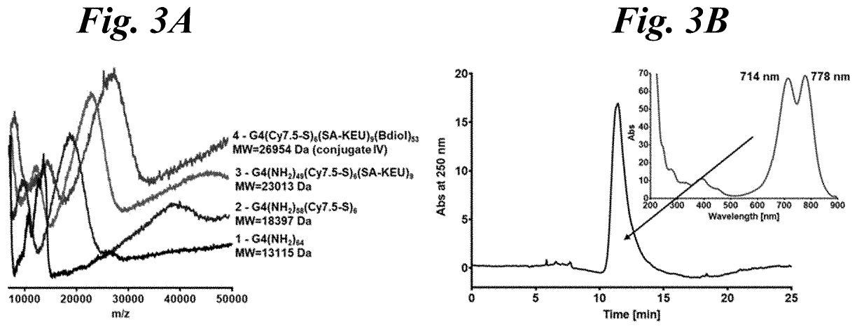

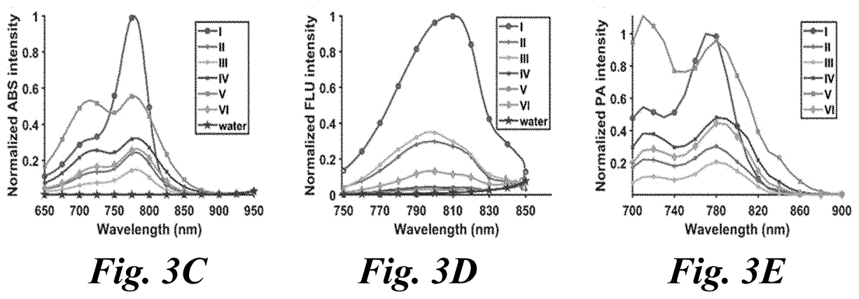

Then, protected KEU was reacted with commercially available DSS to afford NHS ester derivative, followed by removal of tertiary butyl groups that provided NHS-SA-KEU. NHS-SA-KEU was purified on a reverse phase flash chromatography lyophilized, characterized by LC-MS (formula C₂₄H₃₆N₄O₁₂, theoretical molecular weight: 572.56 DA, observed molecular weight: 572.8 Da) and further used for the synthesis of conjugate I, II, III, IV and V. To prepare conjugate I, NHS-SA-KEU was reacted with NE-Boc-L-Lysine, followed by removal of Boc and tertiary butyl protecting groups and conjugation of sulfo-Cy7.5 (FIG. 1A). The conjugate I Cy7.5-K-SA-KEU was purified on a RP-HPLC and mass spectrometry confirmed its molecular mass of 1554 Da. PSMA-targeted dendrimer conjugates were assembled using one-pot synthesis via consecutive surface conjugation of sulfo-Cy7.5 NIR dyes, SA-KEU PSMA targeting moieties and capping of the remaining terminal primary amines with butane-1,2-diol, as presented in FIG. 1B. Average number of conjugated functionalities was determined based on an increase of the molecular weight upon each synthetic step, as measure by MALDI-TOF mass spectrometry (FIG. 3A shows representative spectra obtained for conjugate IV, Table 1). Four PSMA-targeted dendrimer conjugates having on average 2, 4, 6, and 8 Cy7.5 NIR-dyes and from 9 to 12 SA-KEU targeting moieties and terminal butane-1,2-diol functionalities were synthesized. The non-targeted control dendrimer conjugate was formulated by conjugating 6 sulfo-Cy7.5 dyes and capping primary amines with butane-1,2-diol functionalities. All conjugates were analyzed by RP-HPLC to assess their purity and confirm covalent attachment of sulfo-Cy7.5 dyes with dendrimer, as demonstrated by the UV-Vis spectra recorded under the peak of the chromatogram (FIG. 3B shows representative chromatogram and UV-Vis spectrum recorded for conjugate IV). Spectrophotometric, spectrofluorometric and PA properties of all conjugates are demonstrated in FIG. 3C-FIG. 3E. All conjugates were formulated into 600 μL sample containing 2.88-nmol Cy7.5 dye. Then, each sample was equally split into three for spectrophotometric, spectrofluorometric, and PA tests, respectively. To obtain the excitation wavelength for fluorescence evaluation as well as estimating the shape of PA spectra, optical absorbance of all conjugates was first tested on a UIV-Vis spectrophotometer. A range from 650 nm to 950 nm (slightly wider than the tunable stable wavelength range on a Nd:YAG OPO laser, i.e., 700 nm to 900 nm) in step of 1 nm was selected. When the total amount of the conjugated Cy7.5 dye was the same, the maximum optical absorbance of conjugates II, III, IV, V, and VI were 24.4% (781 nm), 14.8% (780 nm), 32.3% (781 nm), 55.5% (778 nm), and 26.3% (781 nm), respectively of the maximum optical absorbance of I (779 nm). Even though in the 750 nm to 800 nm range no dendrimer conjugate had a higher absorbance than I, in longer wavelength range all conjugates outperforms conjugate I, and below 750 nm V has a higher absorbance than I up to 1.8 times. Due to the emission limit on the spectrofluorometer (up to 850 nm), the excitation for spectrofluorometry was set at 715 nm, and the emission was set from 750 nm to 850 nm in step of 1 nm. The normalized fluorescence intensity spectra are included in FIG. 3D and the quenching rates are given in Table 1. Because of the fluorescence quenching, more optical energy is converted to thermal energy so that stronger thermal-elastic expansion is induced as implied in FIG. 3E. Specifically, to avoid the interference of photobleaching wavelengths from 700 nm to 900 nm in step of 10 nm were tested in PA tubing phantom experiment. As listed in Table 1, the ratio of PA intensity of II, III, IV, V, and VI to I at 780 nm are 30.8%, 20.9%, 49.0%, 97.5%, and 45.4%, respectively. Whereas the maximum PA intensity of II, III, IV, V, and VI are 30.3% (780 nm), 20.6% (780 nm), 48.1% (780 nm), 111.1% (710 nm), and 44.6% (780 nm) with respect to I (770 nm). Specially, the PA intensity of conjugate V is higher than I in 700-750 nm and 790-900 nm range, and the ratio can be up to 203.66% (710 nm) and 1257.44% (860 nm), respectively. A higher PA intensity at longer wavelengths is especially significant for medical applications such as prostate cancer detection, since light penetration is a challenge for endorectal optical delivery, while light with longer wavelength suffers less from scattering hence penetrates deeper. Physicochemical properties of all conjugates are collected in Table 1.

TABLE 1
Characteristics of conjugates.

| | # of | # of SA- | # of | MW | QR | RPA₇₈₀ | RPAₘₐₓ | PB | K_{d} |
|---|---|---|---|---|---|---|---|---|---|
| Conjugate | Cy7.5 | KEU | Bdiol | (Da) | (%) | (%) | (%) | (%/sec) | (μM) |
| I | 1 | 1 | 0 | 1554 | N/A | N/A | N/A | | 0.78 |
| II | 2 | 10 | 32 | 21950 | 76.4 | 30.8 | 30.3 | 0.043 | 0.68 |
| III | 4 | 9 | 30 | 23800 | 174.9 | 20.9 | 20.6 | 0.049 | 0.27 |
| IV | 6 | 9 | 53 | 26954 | 4.4 | 49.0 | 48.1 | 0.037 | 0.41 |
| V | 8 | 12 | 52 | 30400 | 1.3 | 97.5 | 111.1 | 0.057 | 0.078 |
| VI | 6 | 0 | 45 | 22590 | 26.6 | 45.4 | 44.6 | | N/A |

Cy7.5-sulfo-cyanine7.5, SA-KEU-suberic acid-lysine-glutamate-urea, Bdiol-butane-1,2-diol, MW-molecular weight as measured by MALDI-TOF, QR-quenching rate, RPA₇₈₀-ratio of photoacoustic intensity at 780 nm, RPAₘₐₓ-ratio of maximum PA intensity in NIR range, K_{d}-dissociation constant, N/A-not-applicable.

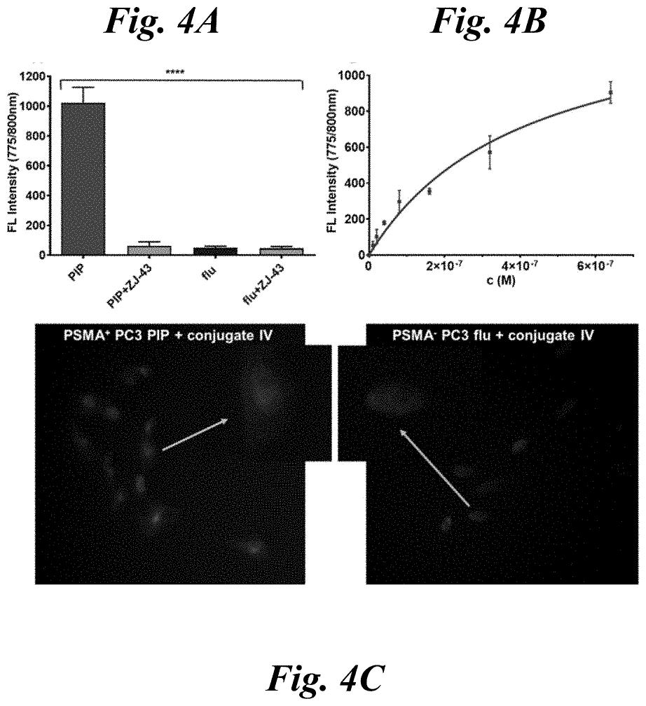

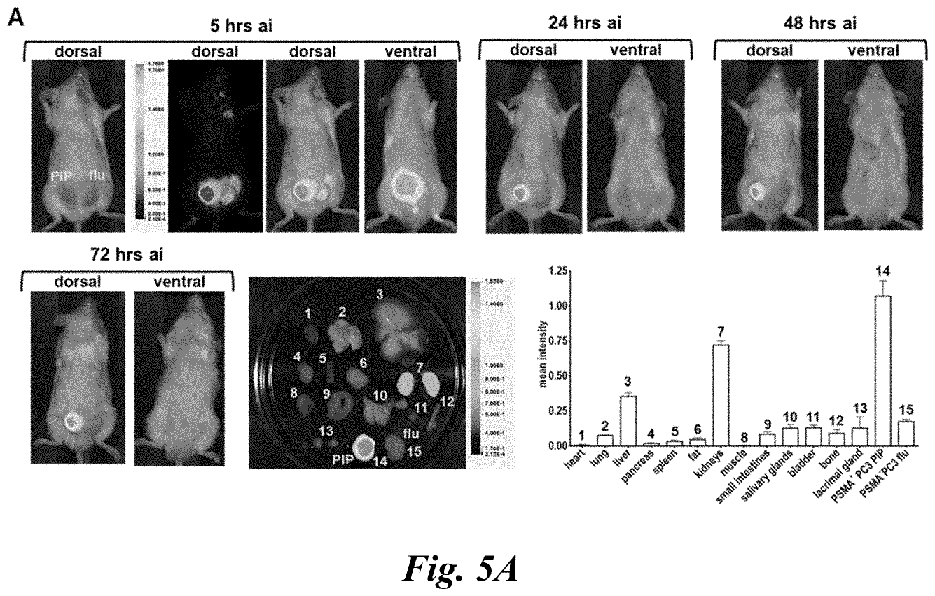

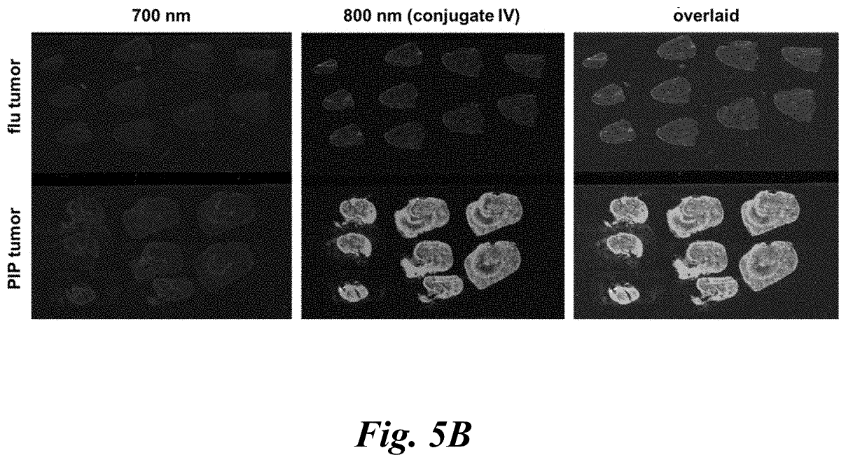

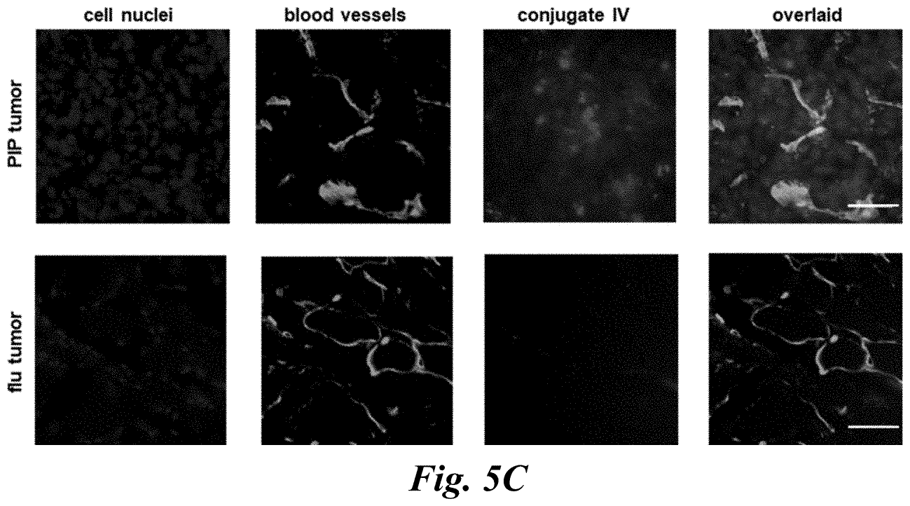

1.7 Evaluation of in vitro specificity. To assess conjugates specificity and affinity to PSMA in vitro, binding assays were carried out using isogenic human prostate cancer PSMA⁺ PC3 PIP and PSMA-PC3 flu cell lines. FIG. 4 displays representative results obtained for conjugate IV, which showed significantly higher uptake by PSMA⁺ PC3 PIP cells compared to PSMA-PC3 flu cells (FIG. 4A). An excess of ZJ-43, commercially available known small molecular weight PSMA antagonist, inhibited the specific uptake of conjugate IV by PSMA⁺ PC3 PIP, confirming its specificity. Conjugate IV also showed a concentration-dependent increase of fluorescence intensity upon elevation of its concentration in PSMA⁺ PC3 PIP cells (FIG. 4B) with a derived dissociation constant (K_{d}) of 0.41 μM (95% confidence interval 0.16-0.66 μM). The conjugate IV also showed significantly higher internalization in PSMA⁺ PC 3 PIP cells compared to PSMA-PC3 flu cells as demonstrate by microscopic evaluation (FIG. 4C). Similar results were obtained for all other PSMA-targeted conjugates and K_{d} value obtained for each conjugate are included in Table 1.
1.8 In vivo evaluation. For in vivo evaluation of specificity and optical and PA contrast properties PSMA-targeted LMW conjugate I and PSMA-targeted dendrimer conjugates IV, and non-targeted control conjugate VI were selected.
1.9 Optical imaging. Conjugates were further evaluated in terms of their potential application in optical imaging in NOD-SCID mice bearing subcutaneous PSMA⁺ PC3 PIP and PSMA-PC3 flu xenografts using a Licor Pearl Impulse Imager in white light and 800 nm channels. FIG. 5 shows representative images collected for mouse injected with 100 μg (3.72 nmol) of conjugate IV equivalent to 22.3 nmol of the Cy7.5 dye. The whole-body imaging indicated highly specific accumulation of the conjugate IV in PSMA⁺ PC3 PIP tumors 5 hrs after injection already. Fluorescence also could be detected in kidneys and bladder (dorsal image) at the same time point. At 24, 48 and 72 h after injection, conjugate IV could be detected only in PSMA⁺ PC3 PIP tumors and ex vivo evaluation of dissected tissues confirmed highest fluorescence intensity in targeted tumors with PSMA⁺ PC3 PIP/PSMA-PC3 flu ratio of 6.2. Relatively lower fluorescent signal also could be detected in kidneys, followed by liver due major renal clearance of conjugate IV and minor hepatic involvement. Evaluation of tumor sections revealed presence of conjugate IV in the entire volume of PSMA⁺ PC3 PIP tumors (FIG. 5B). Microscopic studies involving staining blood vessels (green) and cell nuclei (blue) indicated extravasation of conjugate IV and its cellular internalization (FIG. 5C). Significantly, lower accumulation of conjugate IV was observer in PSMA-PC3 flu tumors that did not allow microscopic evaluation.

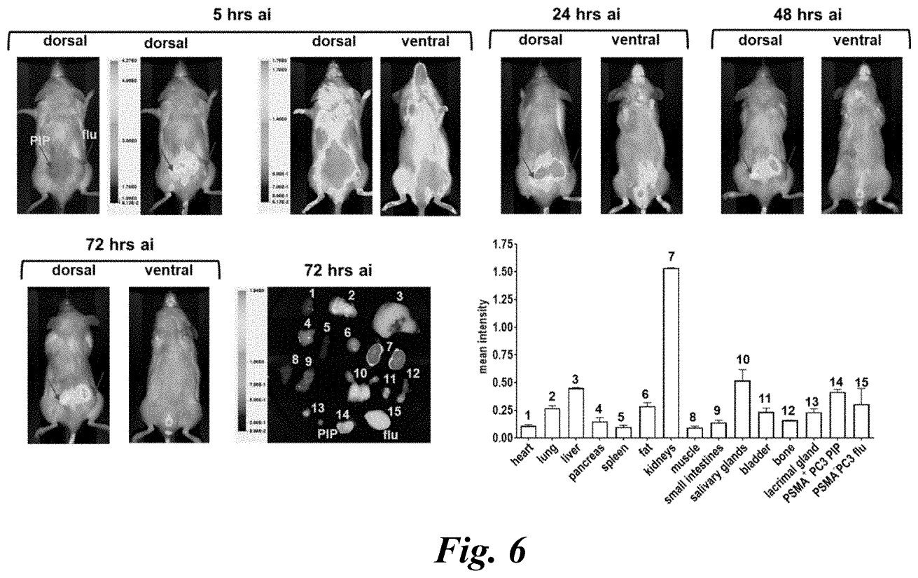

In contrast to PSMA-targeted conjugate IV, control conjugate VI did not show preferential uptake in PSMA⁺ PC3 PIP with its predominant accumulation in kidneys followed by salivary glands, liver (FIG. 6).

1.10 Ultrasound and photoacoustic imaging
1.10.1 Noise models in spectroscopic PA imaging

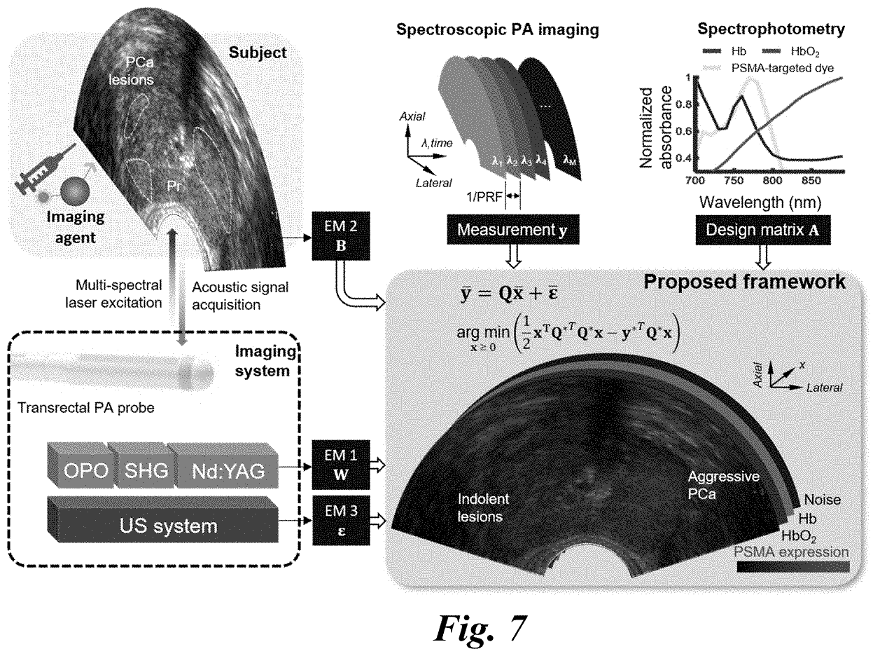

FIG. 7 shows the overall framework of the optimized spectral unmixing for effective spectroscopic PA imaging. There are three major noise sources for spectroscopic PA imaging, which can be concluded as error models as shown in FIG. 7, and as summarized immediately herein below:
EM 1. Laser-dependent error model. The emitted laser energy is heteroscedastic from wavelength to wavelength in the spectral domain (EM 1a) and varies from pulse to pulse in the temporal domain (EM 1b), which makes optical fluence denoted by F fluctuate spectrotemporally. To illustrate, the coefficient of variance (CVs) of F in the near-infrared band (700-900 nm) on our Nd:YAG laser equipped with a tunable optical parametric oscillator (OPO) (Phocus Inline, Opotek Inc., USA) ranges from 3.65% (709 nm) to 9.85% (751 nm).
EM 2. Tissue-dependent spectral distortion model. F is spatially variant due to the heterogenous optical absorption and scattering over light pathways in biological tissues. This “spectral coloring” effect, however, is very difficult to validate in vivo due to the lack of ground truth. In addition, any photobleaching and/or motion artifacts during a sequential spectral scanning may also produce unpredictable spectral inconsistency. These effects altogether generate error-in-variables (EIV) in spectral unmixing. Kim et al., 2013.
EM 3. Ultrasound system noise model. Among various types of electronic noises (i.e., flicker noise, Poisson noise, and Johnson-Nyquist noise), the Johnson-Nyquist noise is known to be the dominant in ultrasound imaging systems. Generally, Johnson-Nyquist noise follows Gaussian distribution in finite positive bandwidth. It is independent of either the optical source (EM 1) or the anatomy (EM 2). Laufer et al., 2010.

To enhance the sensitivity and specificity of the spectral unmixing, the EMs were incorporated into the mathematical model. Suppose there are N endmembers and M different wavelengths (M≥N), the conventional linear mixed model (LMM) considers that

where A ∈ [symbol] ^{M×N} is the design matrix, A(m, n)=I μ_{a,n}(λm)F, λₘ is the m-th wavelength, y ∈R^{M} is the measured PA spectrum, x ∈ R^{N} is the unknown concentration of the endmember, and ε ∈R^{M} is additive measurement noise. Since x is non-negative, the problem is solved by non-negative least squares (NNLS) using quadratic programming techniques, Franc et al., 2005, which is given by

$${argmin}_{x \geq 0} ( \frac{1}{2} {x}^{T} {A}^{T} A x - {y}^{T} A x )$$

To make the least squares problem valid, E must be zero-mean. Besides, to achieve unique solution, columns of A must be linearly independent. Due to the Ems, however, these assumptions are no longer valid. The EMs are addressed as following. To address EM 1, the direct measured spectroscopic PA signal should be compensated as

Y̅=W⁻¹y

where W ∈ R^{M×M} is a diagonal matrix with W_{m,m}=wₘ for m=1, . . . , M, and wₘ is the reciprocal of the pulse energy at wavelength λₘ. Once weighted, y̅ becomes proportional to the optical absorbance which again legitimizes the least squares model. Owing to the stochastic properties of various sources in EM 2, it is modeled as a Gaussian process, where the EIV term is concluded by an additive Gaussian matrix B, i.e., each entry of B is independent and identically distributed (i.i.d.) and satisfies Bᵢⱼ˜ [symbol] (0,σ_{B}²),∀i,j, where N represents Gaussian distribution, such that

[symbol]

y̅=(A+B)x+ε

At last, denote the distribution of the electronic noise in EM 3 as N(xₑ [symbol] ,Σ), where xₑ is the mean value (xₑ>0), [symbol] ∈ [symbol] ^{M} is a vector with all ones, and I is the covariance matrix with all its elements on the diagonal being σₑ². Equivalently, the electronic noise can be separated into a constant offset and a zero-mean Gaussian component such that ε˜xₑ [symbol] +N(0,Σ). Once the spectrotemporal fluctuation in EM 1 is compensated, the distribution of the electronic noise becomes ε˜xₑv+N(0,Σ), where

v=diag(W⁻¹)

Σ̅=W⁻²Σ

By taking the weighted offset as one of the endmembers and rearranging the least squares equation, one gets

$$\overline{y} = [ A + B \vert v ] [ \frac{x}{{x}_{e}} ] + \overline{ε} = Q \overline{x} + \overline{ε}$$

where Q ∈ [symbol] ^{M×(N+1)} is the new design matrix, x̅∈ [symbol] ^{N+1}, and ε̅˜N(0,Σ̅). This modification is referred to as ‘noise segregation’ because it segregates the background electronic noise vxₑ from the other signal components. To convert the overall problem into NNLS model solved by quadratic programming technique, one has

$${argmin}_{\overline{x} \geq 0} ( \frac{1}{2} {\overline{x}}^{T} {Q}^{T} Q \overline{x} - {\overline{y}}^{T} Q \overline{x} )$$

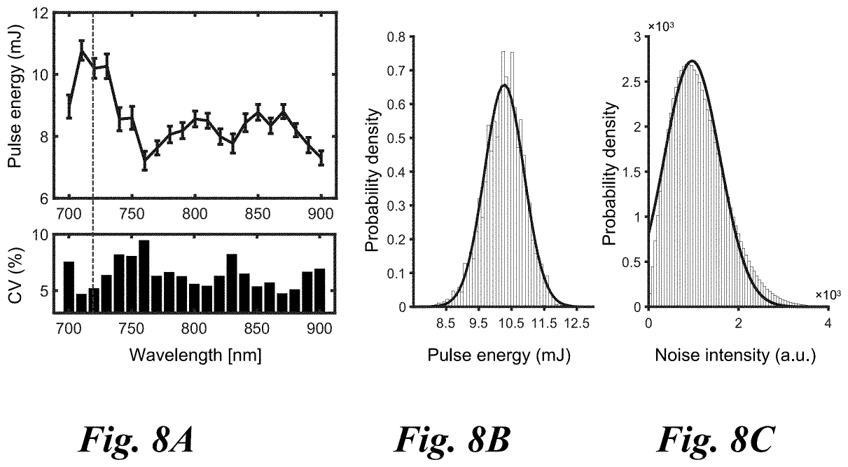

The EMs were calibrated on the presently disclosed research platform as shown in FIG. 8. The spectral and temporal fluctuation of laser emission in EM 1 were calibrated on a tunable Nd:YAG OPO pulsed laser system, where the laser was set in fast-scanning mode with 20 Hz pumping frequency, and each pulse energy was recorded by an energy meter in real-time (PE50BF-DIF-V2, Ophir Photonics, USA). To investigate the temporal fluctuation of the pulse energy, 5,000 sequential emissions were acquired at 720 nm. The probability distribution of the emissions was represented as a histogram and its similarity to a Gaussian distribution was compared. To study the spectral fluctuation, 512 sequential emissions were respectively recorded at wavelengths from 700 nm to 900 nm in 10 nm interval. The change in the mean and the standard deviation (STD) of the pulse energy with respect to the wavelengths were calculated.

EM 3 was calibrated from an L7-4 transducer connected to the Verasonics research package, where 1,344 subsequent frames of PA images (in the size of 256 by 128, axial by lateral), without any ultrasound or laser transmission that contain only electronic noise signals, were acquired. The mean and the STD of all 44,040,192 pixels were calculated, and the intensity distribution was represented as a histogram the similarity of which to a Gaussian distribution was compared. Once those statistics of EM3 were obtained, the spectral signature of the electronic noise (v) and the covariance of ε were derived.

EM 2 was generalized it as a Gaussian process with simplest assumptions and included it as a linear EIV term to the least squares problem. Its impact on wavelength selection in simulations, however, was not observed.

### 1.10.2 Wavelength Optimization

Wavelength optimization provides us an opportunity to use fewer wavelengths to enhance temporal resolution and keep the spectral unmixing accuracy at the meantime. If searched with brute force, the row selection problem is combinatorial in exponential computation time. To avoid such high time complexity, an iterative searching scheme in two stages was proposed. In the first stage, the best N wavelengths that are minimally required for unmixing are included in set S, then in the second stage one wavelength is added to S at a time until the user-defined number of wavelengths (denoted as t) is reached. A detailed workflow is as follows.
Input: number of endmembers N, total number of wavelengths M, user-defined number of wavelengths t, design matrix A, weight v, covariance Σ̅, number of iterations K.
Output: the optimal wavelength subset S.
First stage: Find S that contains Nwavelengths.
   (1) Initialize a counter with (M choose N) bins where each bin points to a permutation.
   (2) Initialize a random fraction of endmembers xo. Generate PA signal y with noises, such that

$$\overline{y} = [ A + B \vert v ] [ \frac{x}{{x}_{e}} ] + \overline{ε} = Q {x}_{0} + \overline{ε} .$$

(3) Solve x=NNLS(Q*, y*) and calculate the root mean squared error (RMSE) for all permutations that have N out of M wavelengths, where Q* and y* contain N out of M rows of Q and y, respectively.
(4) Find the permutation with the smallest RMSE and count one to its bin, where

$$RMSE = \sqrt{\frac{1}{N} {‖ x - {x}_{0} ‖}^{2}}$$

(5) Iterate steps (2)-(4) K times, the permutation with the highest counts is reported as S which is the optimal subset found in this stage.
Second stage: Update S one wavelength at a time.
   (1) Initialize a counter with |S^{c}| bins, where is the cardinality of a set, and S^{C} is the complementary set of S (i.e., S ∪S^{C}={1,2, . . . , M}).
   (2) Generate a random PA signal y̅=Qx₀+ε̅.
   (3) For each element m ∈ S^{C}, solve x=NNLS(Q*, y*) and calculate RMSE, where Q* and y* contain the rows of Q and y indexed by S ∪ {m}, respectively.
   (4) Find the element that yields the smallest RMSE and count one to its bin.
   (5) Iterate steps (2)-(4) K times, then select the element with the highest count and include it into S.
   (6) Iterate (1)-(5) until |S|=t, where t is the desired number of wavelengths.

In terms of computational complexity, the proposed approach is significantly lower compared with exhaustively searching all M choose t permutations. The number of NNLS operations is reduced from O((M choose t)*K) to O(((M choose N)+M²)*K))).

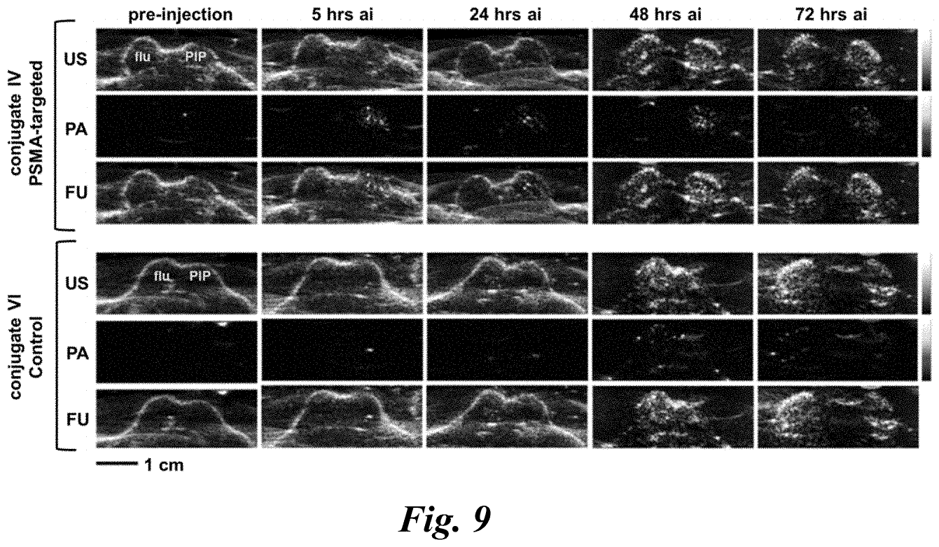

To evaluate potential application of the conjugates in photoacoustic imaging, mice used in optical imaging (described in optical imaging section) were imaged using the Nd:YAG OPO laser and Verasonics Vantage 256 ultrasound research platform. FIG. 9 demonstrates the representative ultrasound (US), PA, and fused images of a transverse plane of subcutaneous PSMA⁺ PC3 PIP and PSMA-PC3 flu xenografts. Under the assumption that different tissue types yield linearly independent PA signal, the two-dimensional (axial and lateral) PA images of the exogeneous imaging agent (conjugate VI) were decomposed from the acquired three-dimensional photoacoustic volume (axial, lateral, and spectral) using non-negative least squares models. The same speed of sound was considered in the beam formation of US and PA imaging. Thus, the two imaging modalities were co-registered by nature. On top of that, image fusion of the two was achieved by using Daubechies 2 wavelet in wavelet transform.

In agreement with optical imaging results, the transverse cross-section in photoacoustic images also confirmed preferential accumulation of conjugate IV in PSMA⁺ PC3 PIP tumors vs. PSMA-PC3 flu tumors with significant contrast allowing for detection of PSMA expression, indicating novel biomedical application for PSMA-targeted dendrimer conjugates. To record background for PA signal mice were imaged before administration of conjugate IV and 5, 24, 48, and 72 h after injection (ai). The average PA intensities within the region-of-interest (ROI) of PSMA⁺ PC3 normalized with respect to the pre-administration baseline PIP were 25.1±11.0, 30.2±21.1, and 21.8±7.4 at 24, 48 and 72 h ai, respectively. The average PA image intensities within the ROI of PSMA-PC3 flu were 4.2±4.3, 5.9±2.5, 3.8±1.3 (a.u.) at the same time points. In contrast to conjugate IV, conjugate VI did not show significant preferable uptake in either PSMA⁺ PC3 PIP or PSMA-PC3 flu. The normalized PA image intensities of PSMA⁺ PC3 PIP tumors were 1.1±0.9, 3.8±4.1, and 1.6±1.4 (a.u.) at 24, 48 and 72 h ai, respectively, and of PSMA-PC3 flu tumors were 2.0±0.9, 3.4±2.4, and 0.9±0.7 (a.u.), respectively.

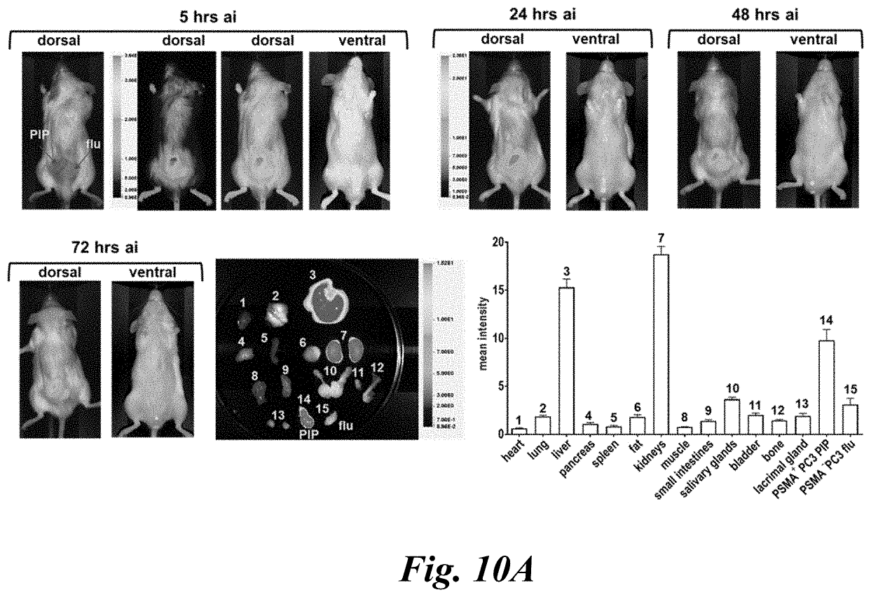

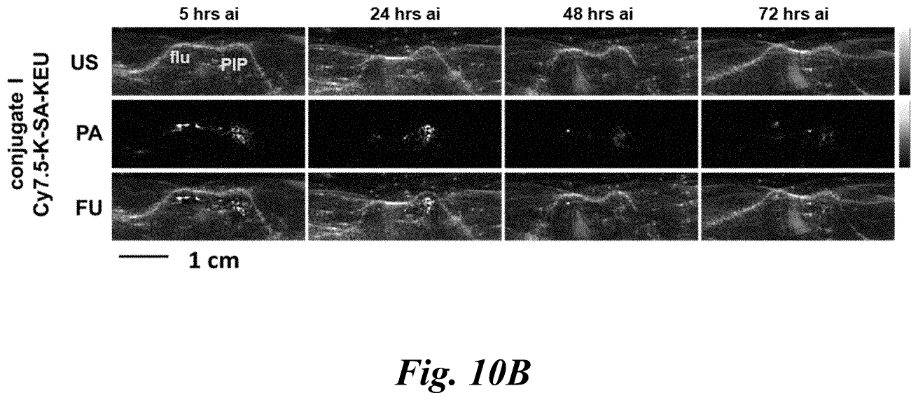

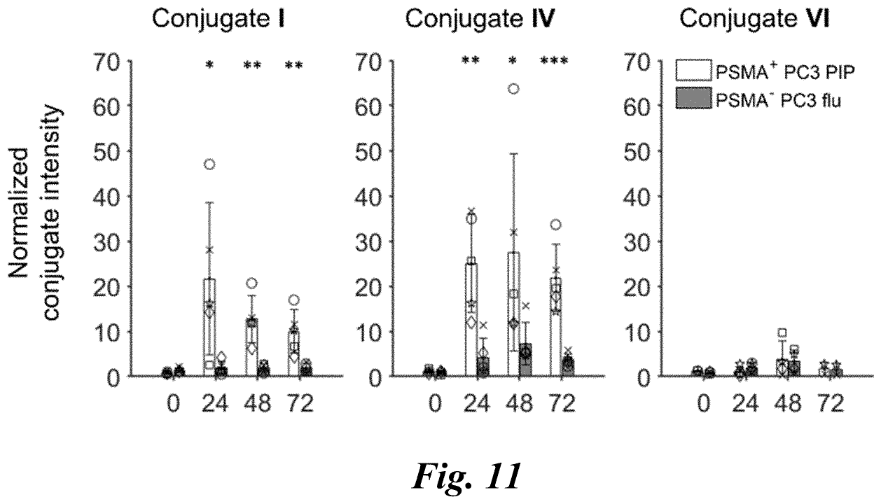

Next, in vivo targeting properties of Cy7.5-K-SA-KEU PSMA were evaluated in NOD-SCID mice bearing subcutaneous PSMA⁺ PC3 PIP and PSMA-PC3 flu tumors. FIG. 10 shows representative optical and PA results obtained for mice injected with 22 nmol of Cy7.5-K-SA-KEU. Although the same number of moles for Cy7.5 in case of conjugate I and IV was injected, optical imaging indicated that much higher fluorescent intensity could be detected in all evaluated tissues in mice treated with conjugate I (compare FIG. 6 and FIG. 10A) due to the lack of fluorescent quenching, which was observed for conjugate IV shown in FIG. 3. Conjugate I also showed preferential accumulation in PSMA⁺ PC3 PIP tumors versus PSMA-PC3 flu tumors, however its highest uptake was detected in kidneys and liver. Due to high accumulation of the conjugate I in PSMA⁺ PC3 PIP tumors, leading to quenching effect it also could be detected in by PA imaging (FIG. 10B), providing opportunity for its application in non-invasive detection of PSMA expression. The corresponding average PA image intensities within the region-of-interest (ROI) of PSMA⁺ PC3 PIP were 21.6±16.8, 12.7±5.2, and 10.0±4.8 (a.u.) at 24, 48 and 72 h ai. The corresponding average PA image intensities within the ROI of PSMA-PC3 flu were 1.9±1.4, 19±0.9, and 2.0±1.1 (a.u.). To compare the PA contrast properties of conjugate I, IV, and VI, the post-injection (24, 48, and 72 h ai) data were normalized with respect to the pre-injection baseline (0 h). The normalized in vivo PA signal intensity in PSMA⁺ PC3 PIP tumors for conjugate I, IV and VI indicated that PSMA-targeted dendrimer conjugated with 6 Cy7.5 NIR dyes showed better PA contrast properties in comparison to LMW agent with 1 Cy7.5 NIR dye and control conjugate VI (FIG. 11).

In summary, the results indicate that developed contrast agents have strong potential for clinical application in minimally invasive detection of PSMA expression in patients with PC with PA imaging for diagnosis, active surveillance, monitoring of therapeutic responses and intra-operative fluorescence surgical guidance for visualization of positive surgical margins that are associated with tumor recurrences and may result in debilitating additional therapies.

### Example 2

Multifunctional PAMAM Dendrimer-Drug Conjugates for Management of Prostate Cancer

### 2.1 Overview

Prostate-specific membrane antigen (PSMA) is a useful biomarker for management of prostate cancer (PC). In the context of PC, PSMA has been leveraged for diagnosis, radiopharmaceutical therapy and targeted drug delivery by antibody-drug conjugates and polylactic acid-polyethylene glycol (PLA-PEG)-based nanoparticles.

Poly(amidoamine) [PAMAM] dendrimers serve as versatile nanoplatforms that can be tailored to different sizes and compositions depending on the application. It has been demonstrated that generation-4 PAMAM dendrimers conjugated with a low-molecular-weight (LMW) PSMA targeted agent, lysine-glutamate-urea (KEU), have suitable pharmacokinetics for targeting of PSMA-expressing tumors with imaging or therapeutic agents. Lesniak et al., 2019. More recently, it has been shown that PSMA-targeted dendrimers conjugated with multiple near-infrared dyes enable detection of PSMA expression with optical and photoacoustic imaging. Lesniak et al., 2021.

The presently disclosed subject matter presents development of multifunctional PSMA-targeted PAMAM dendrimer-drug conjugates (PT-DDCs) and their evaluation in an experimental model of PC.

### 2.2 Methods

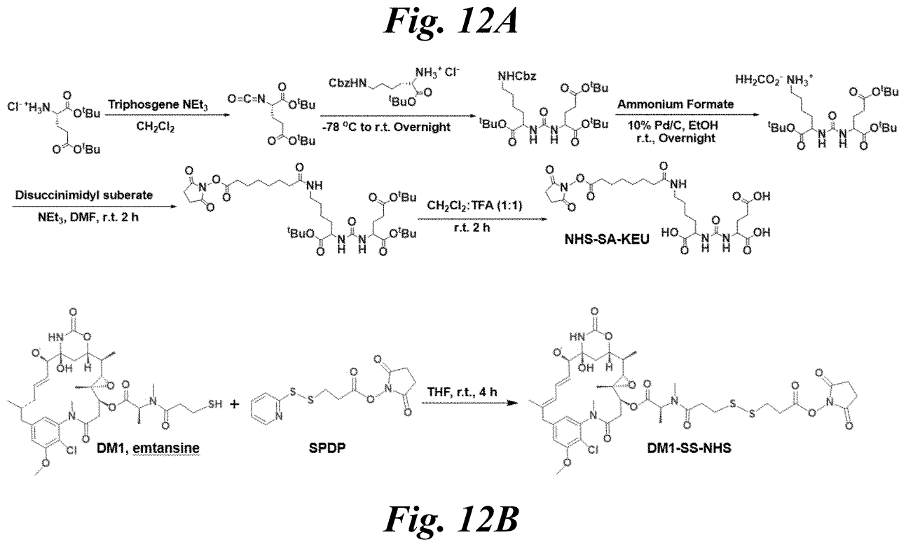

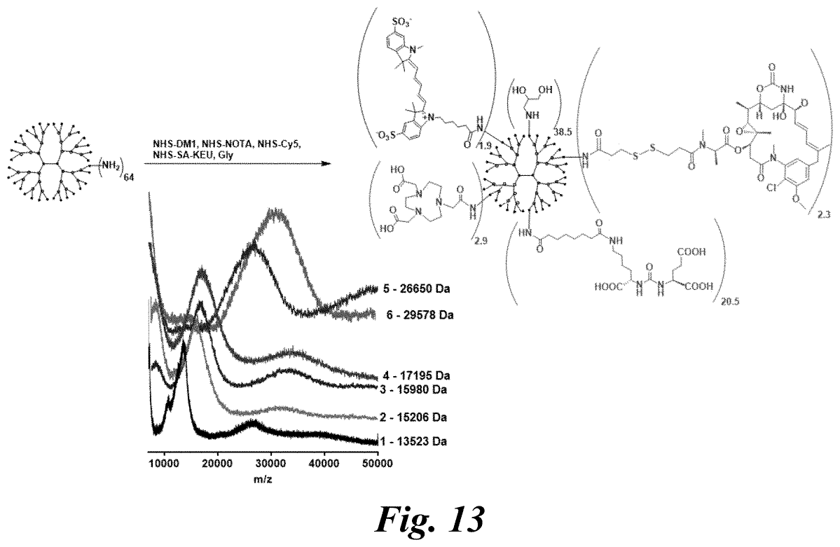

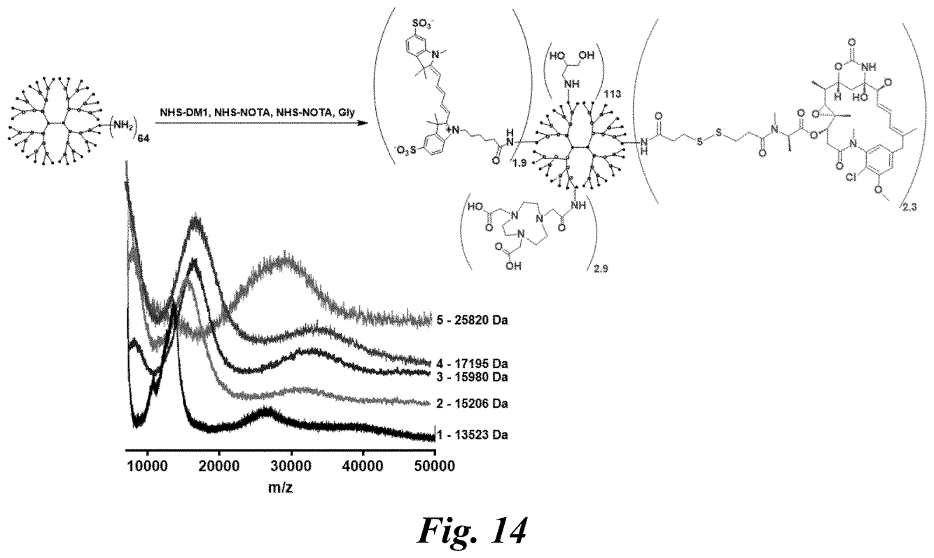

Synthesis of NHS—K-SA-KEU, PSMA-targeting moiety and modification for DM1 for conjugation with dendrimer is illustrated in FIG. 12A and FIG. 12B. The formulation of PT-DDCs is shown in FIG. 13. The PT-DDCs were synthetized through consecutive conjugation of mertansine (DM1, a highly potent antimitotic agent commonly used for formulation of antibody-drug conjugates, Rinnerthaler at al., 2019, Cy5. for optical evaluation, NOTA for radiolabeling with ⁶⁴Cu for PET-CT imaging or ⁶⁷Cu for endoradiotherapy and KEU moieties with generation-4 PAMAM dendrimers. The remaining terminal primary amines were capped with butane-1,2-diol to minimize non-specific in vivo uptake and toxicity of resulting PT-DDCs. The design of PT-DDCs enables comprehensive evaluation of their in vivo properties using optical and nuclear imaging. Non-targeted control dendrimer-drug conjugates (Ctrl-DDC) were formulated without conjugation of KEU (FIG. 14). Physicochemical properties of all DDCs were assessed using high performance liquid chromatography, matrix assisted laser desorption ionization mass spectrometry and dynamic light scattering. PT-DDCs were evaluated using isogenic human prostate cancer PSMA⁺ PC3 PIP and PSMA-PC3 flu cell lines in vitro and in mice bearing the corresponding xenografts.

### 2.3 Results

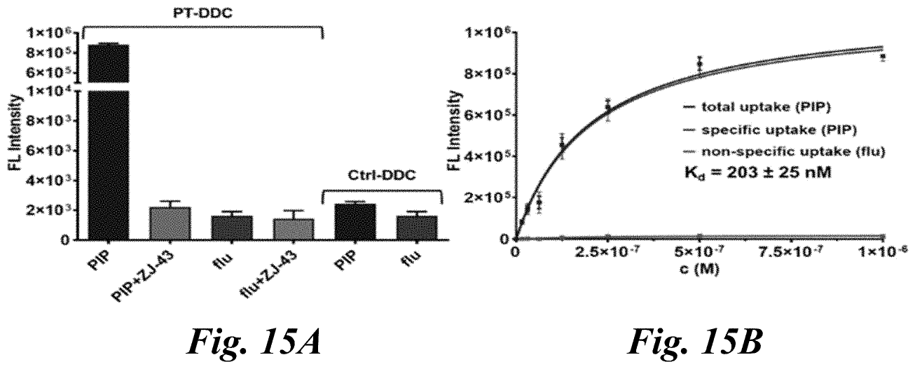

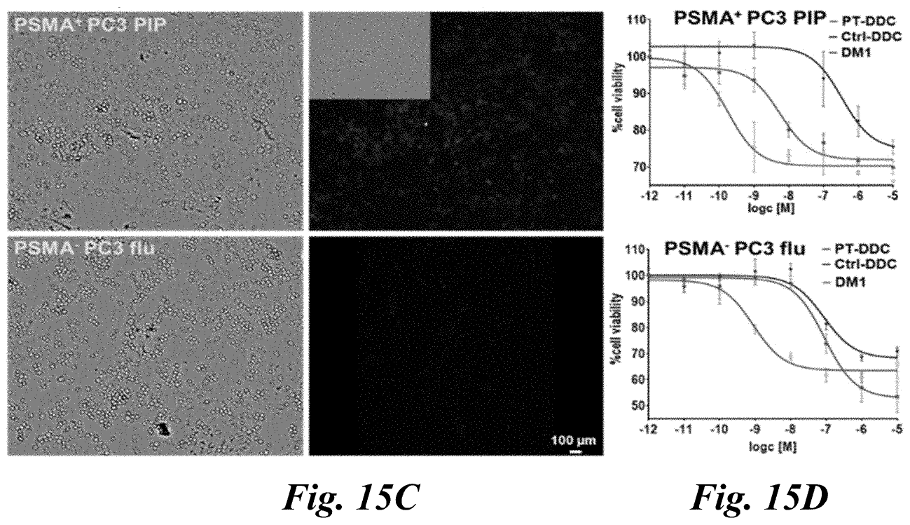

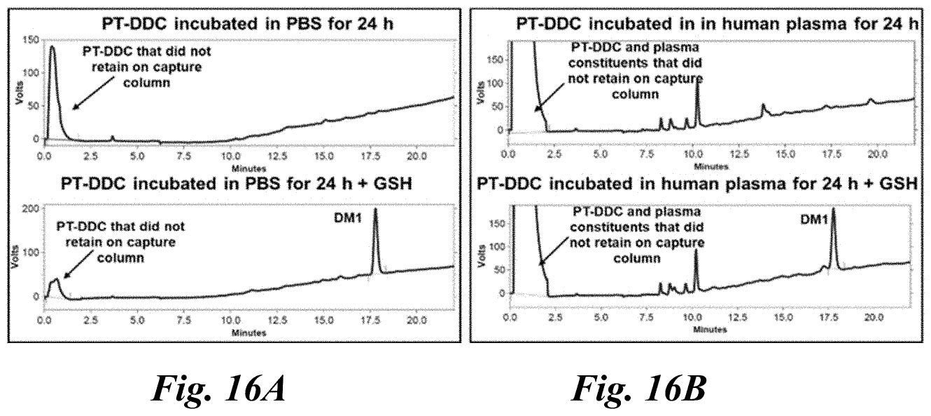

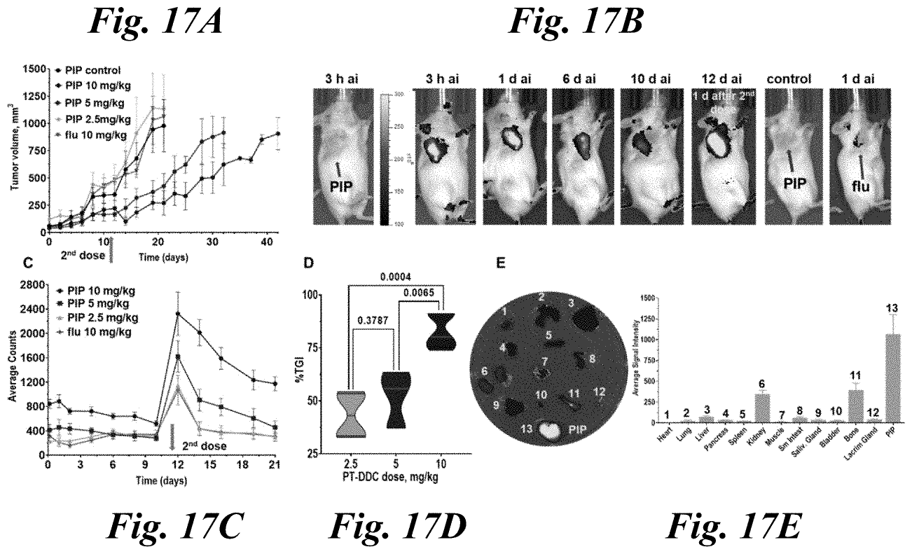

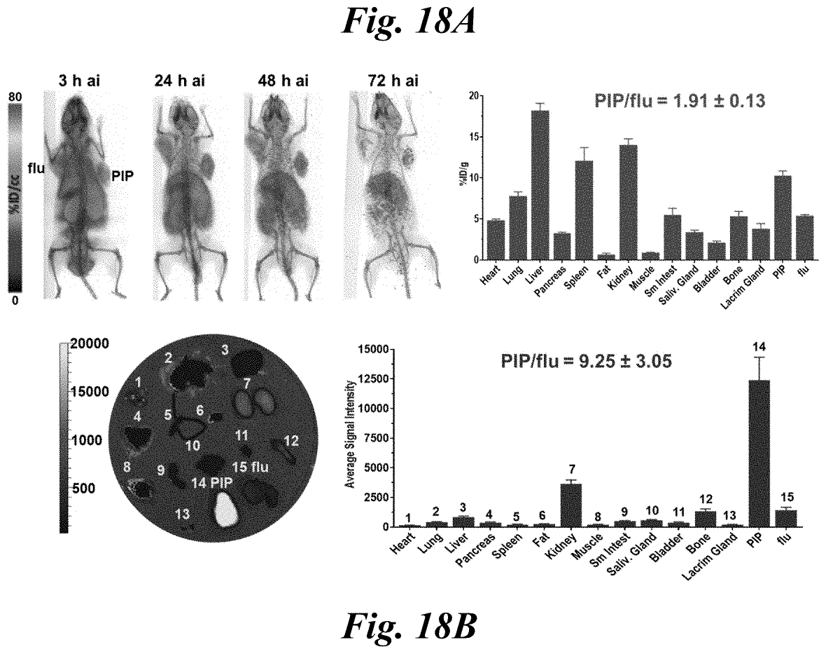

PT-DDCs showed high in vitro uptake by PSMA⁺ PC3 PIP cells that could be blocked by ZJ-43 LMW, a potent PSMA inhibitor (FIG. 15A). Yamamoto et al., 2004. PT-DDCs also showed concentration dependent accumulation and cytotoxicity in PSMA⁺ PC3 PIP cells with KD of 203+25 nM (95% CI 151-254 nM) and IC₅₀ of 4.57 nM (95% CI 2.48-128.4 nM). PT-DDCs did not accumulate in PSMA-PC3 flu cells and showed higher IC₅₀ of 89.9 (95% CI 43.5-185.8 nM) against this cell line (FIG. 15D). Ctrl-DDC did not accumulate in either of cell lines and provided higher IC₅₀ values of 338.8 nM (95% CI 59.5-1,924 nM) for PSMA⁺ PC3 PIP cells and 84.6 nM (95% CI 37.7-189.7 nM) for PSMA-PC3 flu cells. Unmodified DM1 proved potent in both cell lines, demonstrating an IC₅₀ value of 176.5 μM (95% CI 87.6-355.4 μM) and 89.04 μM (95% CI 55.5-995.5 μM) in PSMA⁺ PC3 PIP and PSMA-PC3 flu, respectively. Evaluation of stability indicated that PT-DDCs did not release DM1 in PBS and human blood plasma during 24 h incubation at 37° C. and required addition of glutathione, an intracellular reducing agent (FIG. 16). Therapy studies indicated selective accumulation of PT-DDCs in PSMA⁺ PC3 PIP tumors, as confirmed by non-invasive optical imaging, and dose-dependent tumor growth inhibition (TGI), reaching 82.62 8.25% for 10 mg/kg dose (FIG. 17). Neither accumulation of PT-DDCs in PSMA-PC3 flu tumors nor therapeutic response were detected in mice treated with a 10 mg/kg dose. PET/CT studies in mice bearing PSMA⁺ PC3 PIP and PSMA-PC3 flu tumors confirmed in vivo specificity of PT-DDCs and its potential for concurrent DM1 and radionuclide delivery to PSMA⁺ tumors by showing relatively high uptake of [⁶⁴C] PT-DDC in PSMA⁺ PC3 PIP tumors (FIG. 18.

PT-DDCs also can be used in vivo in relevant PSMA-expressing tumor model systems for theranostic applications.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Ferlay, J.; Soerjomataram, I.; Dikshit, R.; Eser, S.; Mathers, C.; Rebelo, M.; Parkin, D. M.; Forman, D.; Bray, F., Cancer incidence and mortality worldwide: sources, methods and major patterns in GLOBOCAN 2012. Internationaljournal of cancer 2015, 136 (5), E359-86.
Damodaran, S.; Lang, J. M.; Jarrard, D. F., Targeting Metastatic Hormone Sensitive Prostate Cancer: Chemohormonal Therapy and New Combinatorial Approaches. The Journal of urology 2019, 201 (5), 876-885.
James, N. D.; Spears, M. R.; Clarke, N. W.; Deamaley, D. P.; Mason, M. D.; Parker, C. C.; Ritchie, A. W.; Russell, J. M.; Schiavone, F.; Attard, G.; de Bono, J. S.; Birtle, A.; Engeler, D. S.; Elliott, T.; Matheson, D.; O'Sullivan, J.; Pudney, D.; Srihari, N.; Wallace, J.; Barber, J.; Syndikus, I.; Parmar, M. K.; Sydes, M. R.; Investigators, S., Failure-Free Survival and Radiotherapy in Patients With Newly Diagnosed Nonmetastatic Prostate Cancer: Data From Patients in the Control Arm of the STAMPEDE Trial. JAMA oncology 2016, 2 (3), 348-57.
Pinsky, P. F.; Prorok, P. C.; Kramer, B. S., Prostate Cancer Screening—A Perspective on the Current State of the Evidence. The New England journal of medicine 2017, 376 (13), 1285-1289.
Tosoian, J. J.; Trock, B. J.; Landis, P.; Feng, Z.; Epstein, J. I.; Partin, A. W.; Walsh, P. C.; Carter, H. B., Active surveillance program for prostate cancer: an update of the Johns Hopkins experience. Journal of clinical oncology: official journal of the American Society of Clinical Oncology 2011, 29 (16), 2185-90.
Chen, R. C.; Rumble, R. B.; Loblaw, D. A.; Finelli, A.; Ehdaie, B.; Cooperberg, M. R.; Morgan, S. C.; Tyldesley, S.; Haluschak, J. J.; Tan, W.; Justman, S.; Jain, S., Active Surveillance for the Management of Localized Prostate Cancer (Cancer Care Ontario Guideline): American Society of Clinical Oncology Clinical Practice Guideline Endorsement. Journal of clinical oncology: official journal of the American Society of Clinical Oncology 2016, 34 (18), 2182-90.
Mamawala, M. M.; Rao, K.; Landis, P.; Epstein, J. I.; Trock, B. J.; Tosoian, J. J.; Pienta, K. J.; Carter, H. B., Risk prediction tool for grade re-classification in men with favourable-risk prostate cancer on active surveillance. BJU international 2017, 120 (1), 25-31.
Picchio, M.; Mapelli, P.; Panebianco, V.; Castellucci, P.; Incerti, E.; Briganti, A.; Gandaglia, G.; Kirienko, M.; Barchetti, F.; Nanni, C.; Montorsi, F.; Gianolli, L.; Fanti, S., Imaging biomarkers in prostate cancer: role of PET/CT and MRI. European journal of nuclear medicine and molecular imaging 2015, 42 (4), 644-55.
Kiess, A. P.; Banerjee, S. R.; Mease, R. C.; Rowe, S. P.; Rao, A.; Foss, C. A.; Chen, Y.; Yang, X.; Cho, S. Y.; Nimmagadda, S.; Pomper, M. G., Prostate-specific membrane antigen as a target for cancer imaging and therapy. Quarterly Journal of Nuclear Medicine andMolecular Imaging 2015, 59 (3), 241-268.
Maurer, T.; Eiber, M.; Schwaiger, M.; Gschwend, J. E., Current use of PSMA-PET in prostate cancer management. Nature Reviews Urology 2016, 13 (4), 226-235.
Azad, B. B.; Banerjee, S. R.; Pullambhatla, M.; Lacerda, S.; Foss, C. A.; Wang, Y. C.; Ivkov, R.; Pomper, M. G., Evaluation of a PSMA-targeted BNF nanoparticle construct. Nanoscale 2015, 7 (10), 4432-4442.
Chen, Y.; Dhara, S.; Banerjee, S. R.; Byun, Y.; Pullambhatla, M.; Mease, R. C.; Pomper, M. G., A low molecular weight PSMA-based fluorescent imaging agent for cancer. Biochemical and Biophysical Research Communications 2009, 390 (3), 624-629.
Dogra, V.; Chinni, B.; Singh, S.; Schmitthenner, H.; Rao, N.; Krolewski, J. J.; Nastiuk, K. L., Photoacoustic imaging with an acoustic lens detects prostate cancer cells labeled with PSMA-targeting near-infrared dye-conjugates. Journal of biomedical optics 2016, 21 (6), 66019.
Zhang, H. K.; Chen, Y.; Kang, J.; Lisok, A.; Minn, I.; Pomper, M. G.; Boctor, E. M., Prostate-specific membrane antigen-targeted photoacoustic imaging of prostate cancer in vivo. Journal of biophotonics 2018,11 (9), e201800021.
Cheng, K.; Chen, H.; Jenkins, C. H.; Zhang, G.; Zhao, W.; Zhang, Z.; Han, F.; Fung, J.; Yang, M.; Jiang, Y.; Xing, L.; Cheng, Z., Synthesis, Characterization, and Biomedical Applications of a Targeted Dual-Modal Near-Infrared-II Fluorescence and Photoacoustic Imaging Nanoprobe. ACS nano 2017,11 (12), 12276-12291.
Fu, Q.; Zhu, R.; Song, J.; Yang, H.; Chen, X., Photoacoustic Imaging: Contrast Agents and Their Biomedical Applications. Advanced materials 2019, 31 (6), e1805875; (c) Wang, S.; Lin, J.; Wang, T.; Chen, X.; Huang, P., Recent Advances in Photoacoustic Imaging for Deep-Tissue Biomedical Applications. Theranostics 2016, 6 (13), 2394-2413.
de Araujo, R. V.; Santos, S. D.; Ferreira, E. I.; Giarolla, J., New Advances in General Biomedical Applications of PAMAM Dendrimers. Molecules 2018, 23 (11).
Maresca, K. P.; Hillier, S. M.; Femia, F. J.; Keith, D.; Barone, C.; Joyal, J. L.; Zimmerman, C. N.; Kozikowski, A. P.; Barrett, J. A.; Eckelman, W. C.; Babich, J. W., A series of halogenated heterodimeric inhibitors of prostate specific membrane antigen (PSMA) as radiolabeled probes for targeting prostate cancer. J Med Chem 2009, 52 (2), 347-57.
Lesniak, W. G.; Boinapally, S.; Banerjee, S. R.; Behnam Azad, B.; Foss, C. A.; Shen, C.; Lisok, A.; Wharram, B.; Nimmagadda, S.; Pomper, M. G., Evaluation of PSMA-Targeted PAMAM Dendrimer Nanoparticles in a Murine Model of Prostate Cancer. Molecular pharmaceutics 2019, 16 (6), 2590-2604.
Mease, R. C.; Dusich, C. L.; Foss, C. A.; Ravert, H. T.; Dannals, R. F.; Seidel, J.; Prideaux, A.; Fox, J. J.; Sgouros, G.; Kozikowski, A. P.; Pomper, M. G., N-[N-[(S)-1,3-dicarboxypropyl]carbamoyl]-4-[F-18]fluorobenzyl-L-cysteine, [F-18] DCFBC: A new imaging probe for prostate cancer. Clinical Cancer Research 2008, 14 (10), 3036-3043
Kim M, Kang J, Chang J H, Song T, Yoo Y. Image quality improvement based on inter-frame motion compensation for photoacoustic imaging: A preliminary study. 2013 IEEE International Ultrasonics Symposium (IUS), 2013:1528-1531.
Laufer J, Cox B, Zhang E, Beard P. Quantitative determination of chromophore concentrations from 2D photoacoustic images using a nonlinear model-based inversion scheme. Appl Opt. 2010; 49(8):1219-1233.
Franc, V., HlavnL V., Navara, M. (2005). Sequential Coordinate-Wise Algorithm for the Non-negative Least Squares Problem. In: iagalowicz, A., Philips, W. (eds) Computer Analysis of Images and Patterns. CAtP 2005. Lecture Notes in Computer Science, vol 3691, pp 407-414. Springer, Berlin. Heidelberg.
Lesniak, W. G., Wu Y., Kang, J., Boinapally, S., Banerjee, S. R., Lisok, A., Jablonska, A., Boctor, E. M., Pomper, MG., Dual contrast agents for fluorescence and photoacoustic imaging: evaluation in a murine model of prostate cancer, Nanoscale, 2021, 13, 9217-9228.
Rinnerthaler G, Gampenrieder S P, Greil R. HER2 Directed Antibody-Drug-Conjugates beyond T-DM1 in Breast Cancer. Int J Mol Sci. 2019, 20(5):1115.
Yamamoto T, Hirasawa S, Wroblewska B, Grajkowska E, Zhou J, Kozikowski A, Wroblewski J, Neale JH. Antinociceptive effects of N-acetylaspartylglutamate (NAAG) peptidase inhibitors ZJ-11, ZJ-17 and ZJ-43 in the rat formalin test and in the rat neuropathic pain model. Eur J Neurosci. 2004, 20(2):483-94.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 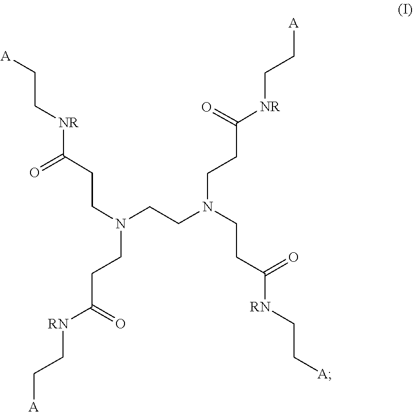
wherein: each A is: 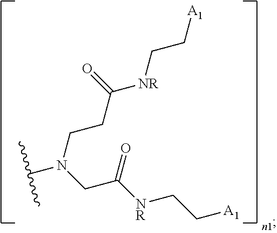
wherein each A₁ is selected from A, a prostate-specific membrane antigen (PSMA) targeting moiety, an optical imaging agent, chelating agent, and/or therapeutic agent, and an end-capping group (EC) or —NH₂; n1 is an integer selected from 1, 2, 3, 4, 5, 6, 7, 8, 9, and 10; and pharmaceutically acceptable salts thereof.

2. The compound of claim 1, wherein the PSMA targeting moiety comprises a Lys-Glu-urea moiety having the following structure: 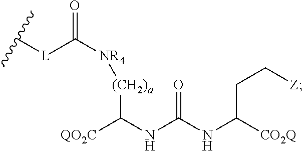
wherein: Z is tetrazole or CO₂Q; Q is 1-1 or a protecting group; a is an integer selected from 1, 2, 3, 4, and 5; R₄ is independently H, substituted or unsubstituted C₁-C₄ alkyl, or —CH₂-R₅; R₅ is selected from substituted or unsubstituted aryl and substituted or unsubstituted heteroaryl; and L is a linker.

3. The compound of claim 1, wherein the linker (L) is selected from —(CH₂)ₘ₁—, —C(═O)-(CH₂)ₘ₁—, —NR₃—C(═O)-(CH₂)ₘ₁—, —(CH₂—CH₂—O)ₜ—, —C(═O)—(CH₂—CH₂—O)ₜ₁—, —(O—CH₂—CH₂)ₜ-,—C(═O)-(O—CH₂—CH₂)ₜ₁—, —C(═O)-(CHR₂)ₘ₁—NR₃—C(═O)-(CH₂)ₘ₁—, —C(═O)-(CH₂)ₘ₁—O—C(═O)—NR₃—(CH₂)ₚ₁—, —C(═O)-(CH₂)ₘ₁—NR₃—C(═O)—O—CH₂)ₚ₁—, —C(═O)-(CH₂)ₘ—NR₃—C(═O)—NR₃—(CH₂)ₚ—, —C(═O)-(CH₂)ₘ—NR₃—C(═O)-(CH₂)ₚ₁—, —C(═O)-(CH₂)ₘ₁—C(═O)—NR₃—(CH₂)ₚ₁—, —C(═O)-(CH₂)ₘ₁—NR₃—C(═O)—NR₃—(CH₂)ₚ₁—, —C(═O)-(CH₂)ₘ₁—NR₁—C(═O)—NR₃—(CH₂)ₚ₁—, —C(═O)-(CH₂)ₘ₁—O—C(═O)—NR₃—,—C(═O)—CH₂)ₘ₁—O—C(═O)—NR₃—(CH₂)ₚ₁—,—C(═O)-(CH₂)ₘ₁—NR₃—C(═O)—O—(CH₂)ₚ₁—, polyethylene glycol, glutaric anhydride, albumin, and one or more amino acids; where each R is independently selected from H and C₁-C₄ alkyl; each R¹ is independently selected from H, Na⁺, C₁-C₄ alkyl, and a protecting group; each R₂ is independently selected from hydrogen, and —COOR₁; each R₃ is independently selected from hydrogen, substituted or unsubstituted linear or branched alkyl, alkoxyl, substituted or unsubstituted cycloalkyl, substituted or unsubstituted cycloheteroalkyl, substituted or unsubstituted aryl, substituted or unsubstituted heteroaryl, substituted or unsubstituted arylalkyl, and substituted or unsubstituted heteroarylalkyl; m1 and p1 are each independently an integer selected from 0, 1, 2, 3, 4, 5, 6, 7 and 8; t1 is an integer selected from 1, 2, 3, 4, 5, 6, 7, 8, 9, 10, 11 and 12.

4. The compound of claim 1, wherein the optical imaging agent comprises a fluorescent dye.

5. The compound of claim 4, wherein the fluorescent dye comprises a cyanine dye.

6. The compound of claim 5, wherein the cyanine dye is selected from: 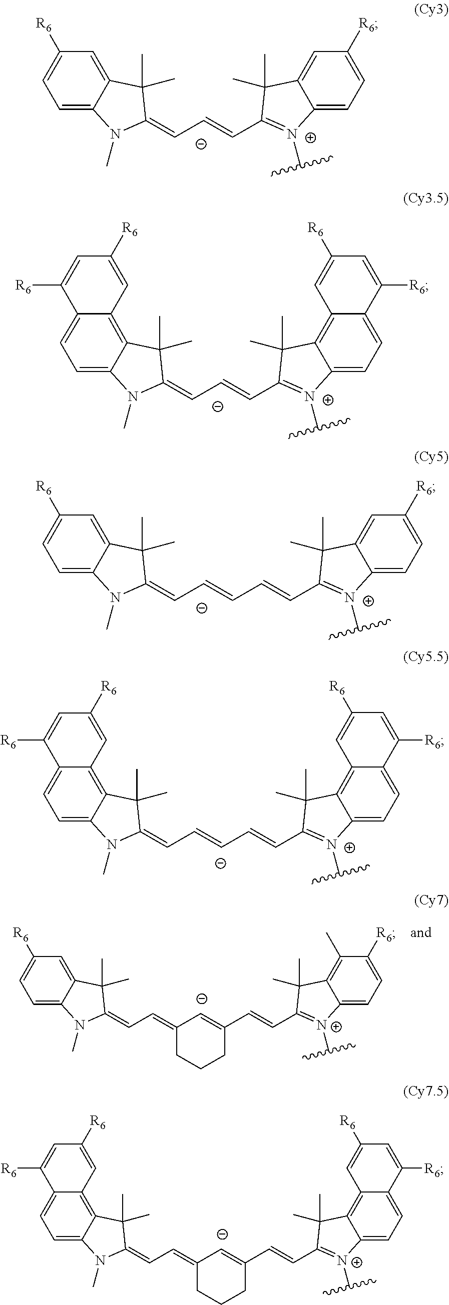
wherein each R₆ can independently be H or —SO₃M, wherein is absent, e.g., R₆ is —SO₃—, hydrogen, sodium, or potassium.

7. The compound of claim 6, wherein the fluorescent dye is Cy7.5: 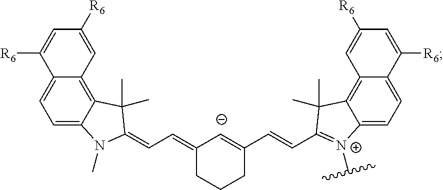
wherein each R₆ can independently be H or —SO₃M, wherein is absent, e.g., R₆ is —SO₃—, hydrogen, sodium, or potassium.

8. The compound of claim 1, wherein the chelating agent is selected from DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH₂—Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid)), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid), (NOTAGA) 1,4,7-triazonane-1,4-diyl)diacetic acid, DFO (Deferoxamine), NETA ([4-{2-(bis-carboxymethylamino)-ethyl]-7-carboxymethl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6] eicosane-1,8-diamine), Sarar (1-N-(4-aminobenzyl)-3,6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and BaBaSar.

9. The compound of claim 1, wherein the chelating agent is selected from:

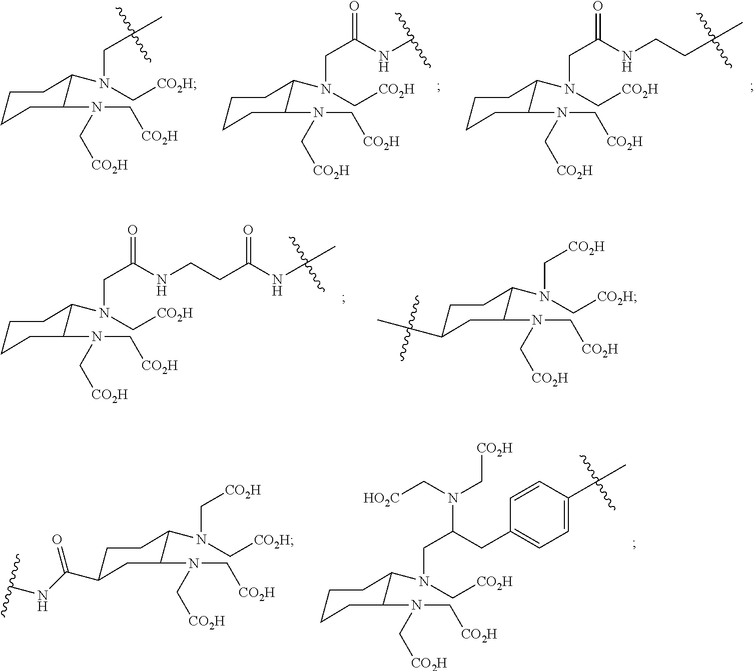

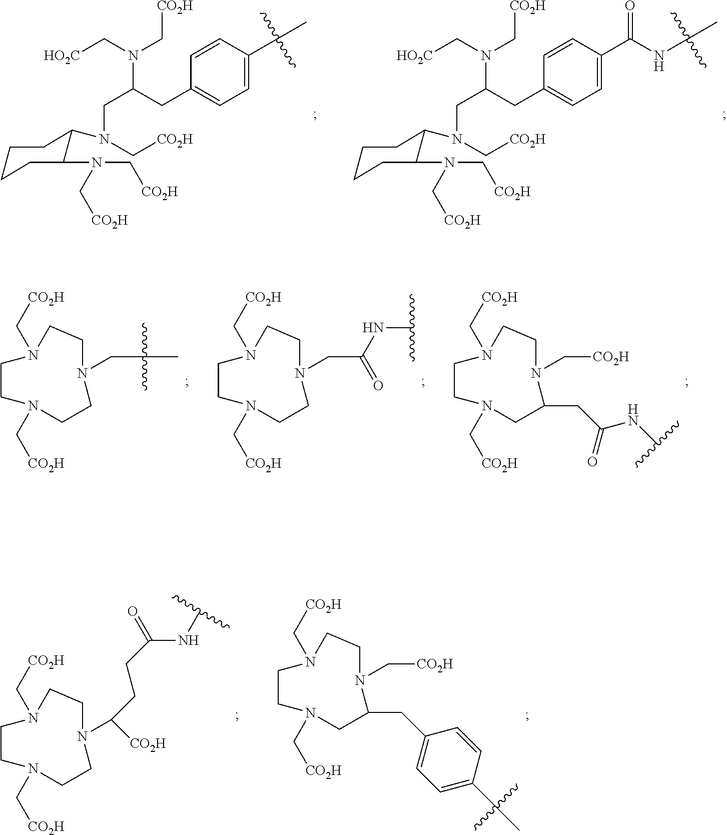

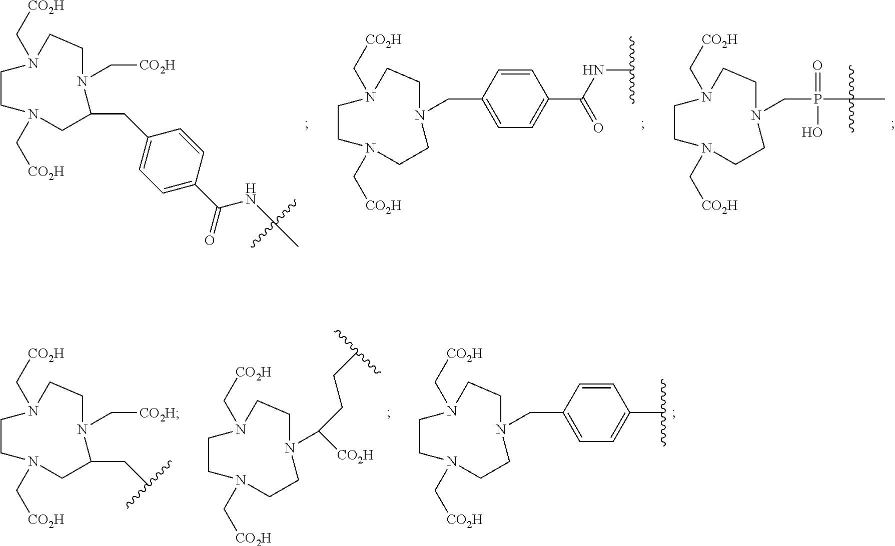 [SMILES: *NC(=O)c1ccc(CC2CN(CC(=O)O)CCN(CC(=O)O)CCN2CC(=O)O)cc1 ; *NC(=O)c1ccc(CN2CCN(CC(=O)O)CCN(CC(=O)O)CC2)cc1 ; *P(=O)(O)CN1CCN(CC(=O)O)CCN(CC(=O)O)CC1 ; *CC1CN(CC(=O)O)CCN(CC(=O)O)CCN1CC(=O)O ; *CCC(C(=O)O)N1CCN(CC(=O)O)CCN(CC(=O)O)CC1 ; *c1ccc(CN2CCN(CC(=O)O)CCN(CC(=O)O)CC2)cc1]

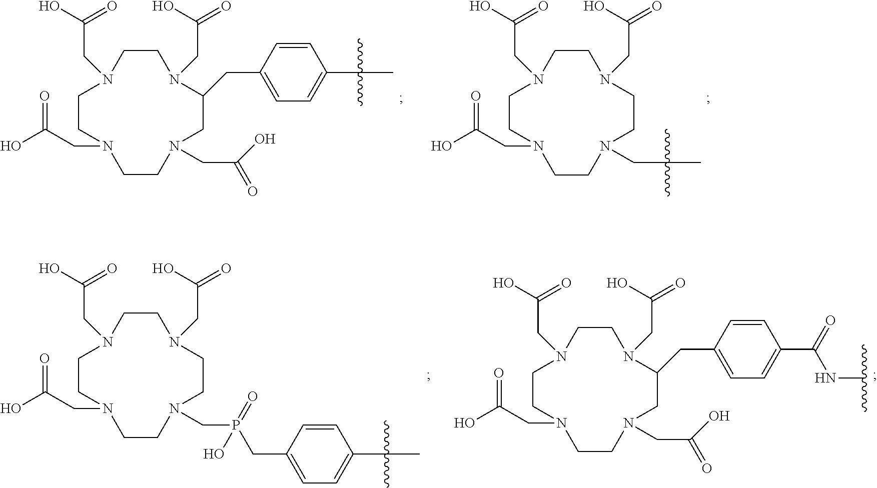 [SMILES: *c1ccc(CC2CN(CC(=O)O)CCN(CC(=O)O)CCN(CC(=O)O)CCN2CC(=O)O)cc1 ; *CN1CCN(CC(=O)O)CCN(CC(=O)O)CCN(CC(=O)O)CC1 ; *c1ccc(CP(=O)(O)CN2CCN(CC(=O)O)CCN(CC(=O)O)CCN(CC(=O)O)CC2)cc1 ; *NC(=O)c1ccc(CC2CN(CC(=O)O)CCN(CC(=O)O)CCN(CC(=O)O)CCN2CC(=O)O)cc1]

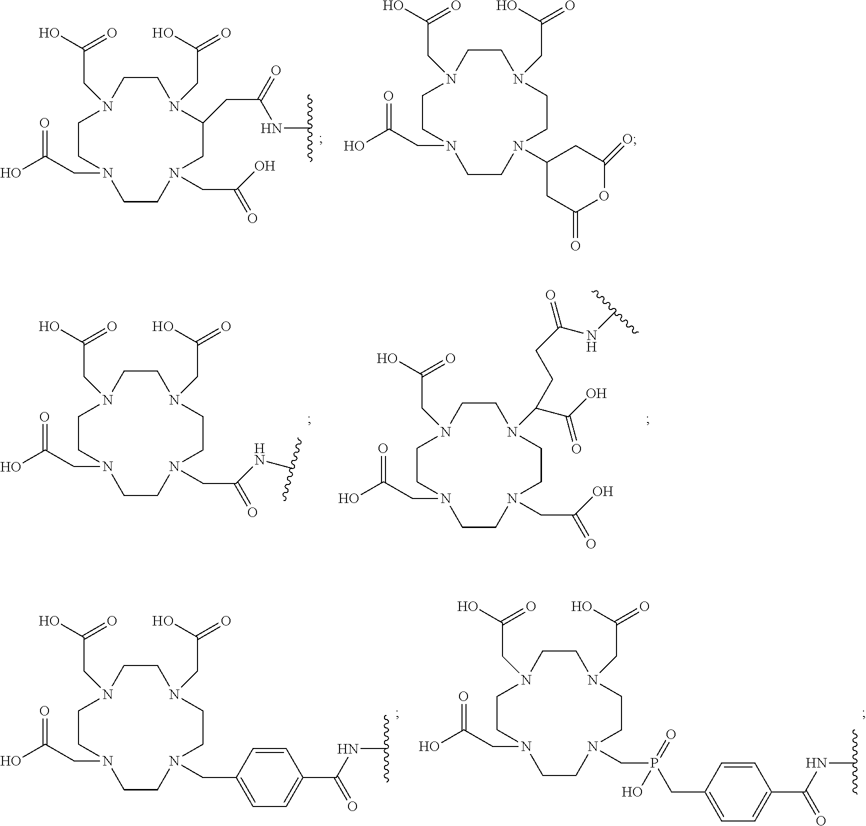 [SMILES: *NC(=O)CC1CN(CC(=O)O)CCN(CC(=O)O)CCN(CC(=O)O)CCN1CC(=O)O ; O=C(O)CN1CCN(CC(=O)O)CCN(C2CC(=O)OC(=O)C2)CCN(CC(=O)O)CC1 ; *NC(=O)CN1CCN(CC(=O)O)CCN(CC(=O)O)CCN(CC(=O)O)CC1 ; *NC(=O)CCC(C(=O)O)N1CCN(CC(=O)O)CCN(CC(=O)O)CCN(CC(=O)O)CC1 ; *NC(=O)c1ccc(CN2CCN(CC(=O)O)CCN(CC(=O)O)CCN(CC(=O)O)CC2)cc1 ; *NC(=O)c1ccc(CP(=O)(O)CN2CCN(CC(=O)O)CCN(CC(=O)O)CCN(CC(=O)O)CC2)cc1]

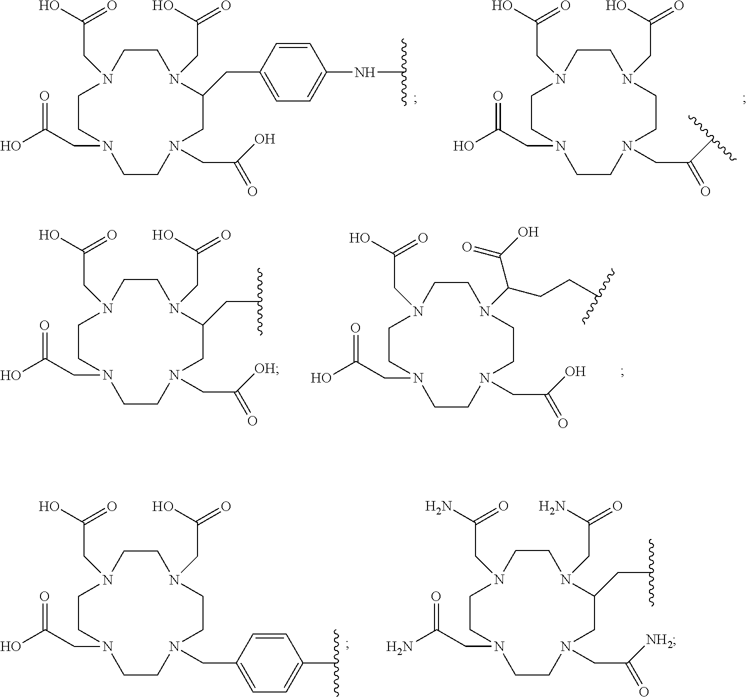 [SMILES: *Nc1ccc(CC2CN(CC(=O)O)CCN(CC(=O)O)CCN(CC(=O)O)CCN2CC(=O)O)cc1 ; *C(=O)CN1CCN(CC(=O)O)CCN(CC(=O)O)CCN(CC(=O)O)CC1 ; *CC1CN(CC(=O)O)CCN(CC(=O)O)CCN(CC(=O)O)CCN1CC(=O)O ; *CCC(C(=O)O)N1CCN(CC(=O)O)CCN(CC(=O)O)CCN(CC(=O)O)CC1 ; *c1ccc(CN2CCN(CC(=O)O)CCN(CC(=O)O)CCN(CC(=O)O)CC2)cc1 ; *CC1CN(CC(N)=O)CCN(CC(N)=O)CCN(CC(N)=O)CCN1CC(N)=O]

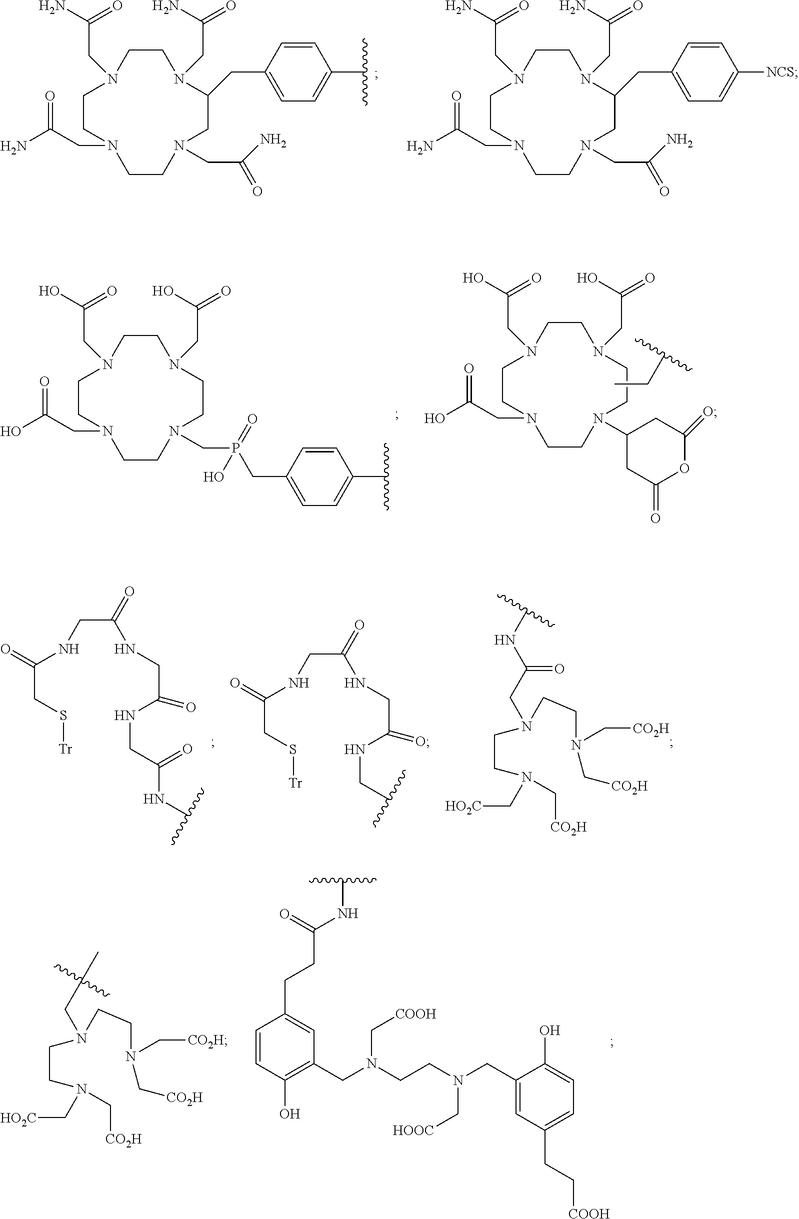

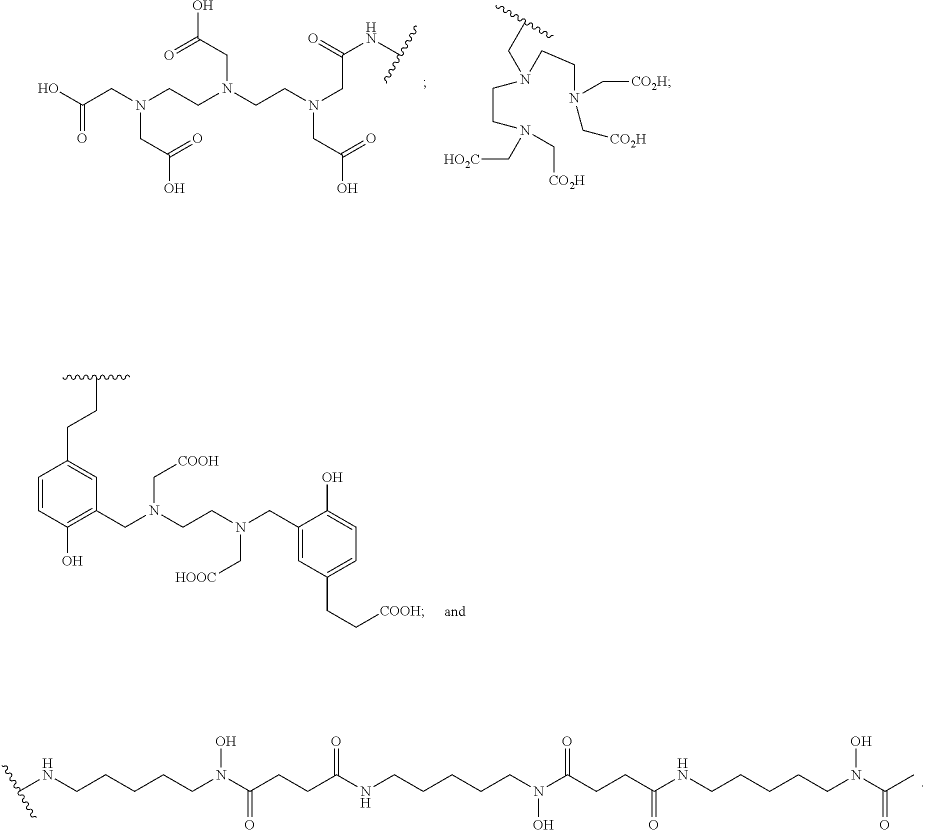 [SMILES: *NC(=O)CN(CCN(CCN(CC(=O)O)CC(=O)O)CC(=O)O)CC(=O)O ; *CN(CCN(CC(=O)O)CC(=O)O)CCN(CC(=O)O)CC(=O)O ; *CCc1ccc(O)c(CN(CCN(CC(=O)O)Cc2cc(CCC(=O)O)ccc2O)CC(=O)O)c1 ; *NCCCCCN(O)C(=O)CCC(=O)NCCCCCN(O)C(=O)CCC(=O)NCCCCCN(O)C(C)=O]

10. The compound of claim 8, wherein the chelating agent comprises NOTA.

11. The compound of claim 1, wherein the chelating agent further comprises a radiometal selected from of ⁶⁰Cu, ⁶²Cu, ⁶⁴Cu, ⁶⁷Cu, ²⁰³Pb, ²¹²Pb, ²²⁵Ac, ¹⁷⁷Lu, ^{99m}Tc, ⁶⁸Ga ¹⁴⁹Tb ⁸⁶y, ⁹⁰y ¹¹¹In ¹⁸⁶Re, ¹⁸⁸Re, ¹⁵³Sm, ⁸⁹Zr, ²¹³Bi, ²¹²Bi, ²¹²Pb, ⁶⁷Ga ⁴⁷Sc, A₁¹⁸F, and ¹⁶⁶Ho.

12. The compound of claim 11, wherein the radiometal is selected from ⁶⁴Cu and ⁶⁷Cu.

13. The compound of claim 1, wherein the therapeutic agent comprises an anti-cancer or chemotherapeutic agent.

14. The compound of claim 13, wherein the therapeutic agent is selected from maytansine, ansamitocin, mertansine/emtansine (DM1), ravtansine/soravtansine (DM4), monomethyl auristatin e, methotrexate, doxorubicin, and paclitaxel.

15. The compound of claim 1, wherein the end-capping group (EC) is selected from —NH₂, —(CH₂)ₘ₁—CH₂—CH(OR₁)-(CH₂)ₘ₁—OR₁, —NR—(CH₂)ₘ₁—CH(OR₁)—(CH₂)ₘ₁—OR₁, —NR—C(═O)—CH₃, —C(═O)—O—Na⁺, —C(═O)—NR—(CH₂)ₘ₁—OR₁, —NR—C(═O)—(CH₂)ₘ₁—C(═O)OR₁, and —NR—(CH₂)ₘ₁—CH(OR₁)-(CH₂)ₘ₁—CH₃; wherein: each R is independently selected from H and C₁-C₄ alkyl; each R₁ is independently selected from H, Na⁺, C₁-C₄ alkyl, and a protecting group; and each m₁ is independently an integer selected from 0, 1, 2, 3, 4, 5, 6, 7, 8, 9, 10, 11, and 12.

16. The compound of claim 15, wherein the end-capping group is —NH—CH₂—CH(OH)—CH₂—OH.

17. The compound of claim 1, comprising a poly(amidoamine) (PAMAM) dendrimer having the following chemical structure: [symbol]
wherein: m, n, p, q, and t are each independently integers from 0 to 64; EC is an end-capping group; IA is an optical imaging agent; and PSMA is a PSMA-targeting moiety.

18. The compound of claim 17, wherein the compound of formula I is: 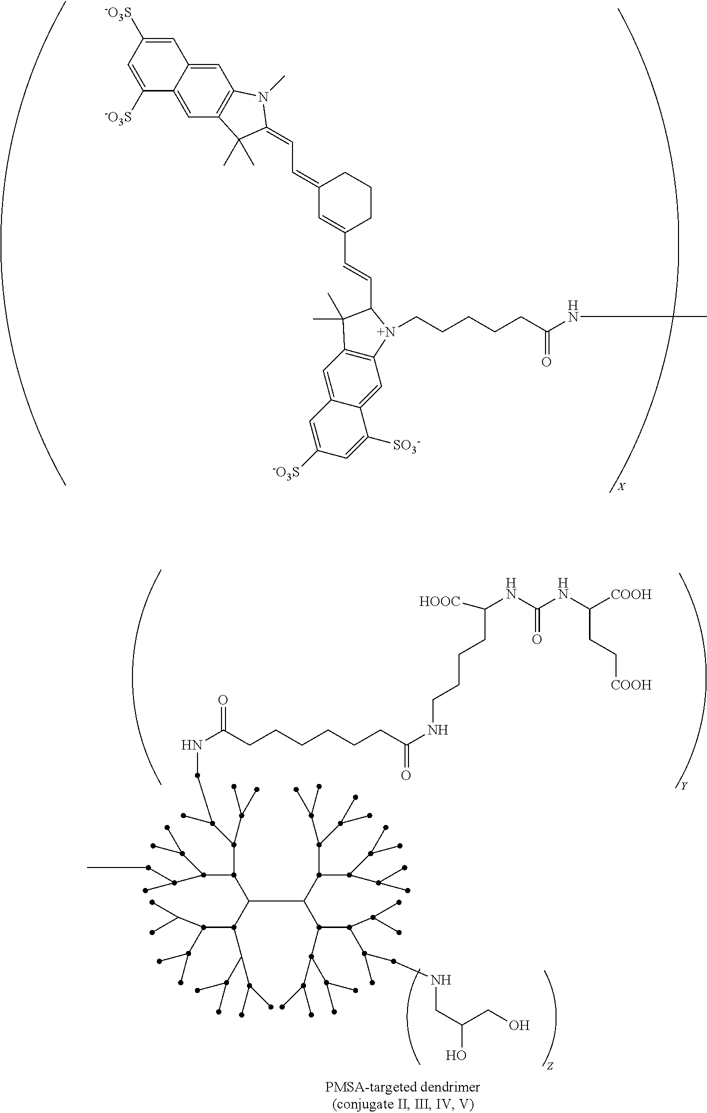
wherein:
X is selected from 1, 2, 3, 4, 5, 6, 7, 8, 9, 10, 11, 12, 13, 14, 15, 16, 17, 18, 19, and 20; Y is selected from 1, 2, 3, 4, 5, 6, 7, 8, 9, 10, 11, 12, 13, 14, 15, 16, 17, 18, 19, and 20; and Z is an integer selected from 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60.

19. A compound of formula (II): 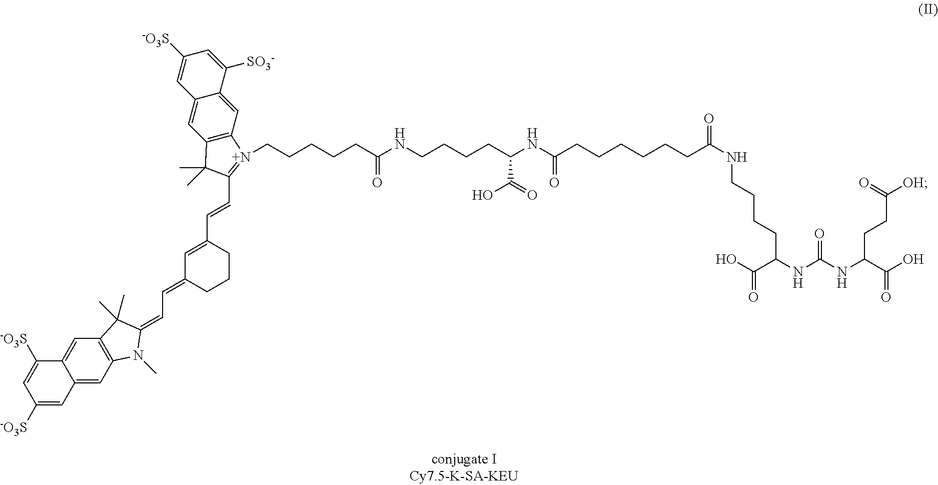
and pharmaceutically acceptable salts thereof.

20. A pharmaceutical formulation comprising the compound of claim 1 and a pharmaceutically acceptable carrier, diluent, or excipient.

21. A method for imaging and/or treating one or more PSMA-expressing expressing tumors or cells, the method comprising contacting the one or more PSMA expressing tumors or cells with an effective amount of a compound of claim 1, or a pharmaceutical formulation thereof and, if a method for imaging, taking an image.

22. The method of claim 21, wherein the imaging is selected from optical imaging, photoacoustic imaging, and positron emission tomography/computed tomography (PET/CT) imaging.

23. The method of claim 21, further comprising diagnosing and/or treating, based on the image, a disease or condition in a subject.

24. The method of claim 21, further comprising monitoring, based on the image, progression or regression of a disease or condition in a subject.

25. The method of claim 21, wherein the method comprises imaging and/or treating a cancer.

26. The method of claim 25, wherein the cancer is selected from prostate cancer, renal cancer, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, adenomas, and tumor neovasculature.
